(19) United States
(12) Patent Application Publication
(10) Pub. No.: US 2026/0257073 A1
(43) Pub. Date: Sep. 3, 2026

(54) ELECTRODE PLACEMENT VERIFICATION SYSTEM

(71) Applicant: ZOLL Medical Corporation, Chelmsford, MA (US)

(72) Inventors: Nicholas J. Chernansky, Oakmont, PA (US); Nathan J. Berry Ann, Cranberry Township, PA (US)

(21) Appl. No.: 19/537,206

(22) Filed: Feb. 11, 2026

Related U.S. Application Data
(60) Provisional application No. 63/757,567, filed on Feb. 12, 2025.

(51) Int. Cl. A61N 1/39; A61N 1/04; A61N 1/36

(52) U.S. Cl. CPC A61N 1/3925; A61N 1/046; A61N 1/048; A61N 1/36031; A61N 1/3904; A61N 1/3993

(57) ABSTRACT

A wearable cardiac device includes a garment configured to be worn on a torso of the ambulatory patient and has one or more therapy electrode pockets. The device may also include a plurality of ECG sensing electrodes configured to sense ECG signals of the ambulatory patient, a plurality of therapy electrodes configured to deliver one or more therapeutic shocks to the ambulatory patient, and a plurality of anatomical placement accelerometers. A controller in electrical communication with the plurality of electrodes and the plurality of anatomical placement accelerometers can be configured to determine, based on received anatomical placement motion signals, at least one of (a) a facing orientation of the plurality of therapy electrodes, or (b) a relative position of each therapy electrode relative to another therapy electrode of the plurality of therapy electrodes, and generate an improper electrode placement in garment alert for the patient.

### CROSS-REFERENCE TO RELATED APPLICATIONS

This application claims priority under 35 USC § 119(e) to U.S. Patent Application Ser. No. 63/757,567, filed on Feb. 12, 2025, the entire contents of which are hereby incorporated by reference.

### TECHNICAL FIELD

The present disclosure is directed towards an electrode placement verification system for cardiac devices.

### BACKGROUND

There are a wide variety of electronic and mechanical devices for monitoring and treating patients' medical conditions. In some examples, depending on the underlying medical condition being monitored or treated, medical devices such as cardiac monitors or defibrillators are prescribed to be externally worn by the patient. In some examples, physicians may use medical devices alone or in combination with drug therapies to treat conditions such as cardiac arrhythmias.

One of the deadliest cardiac arrhythmias is ventricular fibrillation, which occurs when normal, regular electrical impulses are replaced by irregular and rapid impulses, causing the heart muscle to stop normal contractions and to begin to quiver. Normal blood flow ceases, and organ damage or death can result in minutes if normal heart contractions are not restored. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (e.g., the heart stops all electrical activity) result in the heart providing insufficient levels of blood flow to the brain and other vital organs for the support of life.

Cardiac arrest and other cardiac health ailments are a major cause of death worldwide. Various resuscitation efforts aim to maintain the body's circulatory and respiratory systems during cardiac arrest in an attempt to save the life of the patient. The sooner these resuscitation efforts begin, the better the patient's chances of survival. Wearable cardiac medical devices including wearable defibrillators or wearable cardioverter defibrillators can improve the ability to treat these otherwise life-threatening conditions in timely fashion. Such devices operate by applying corrective electrical pulses directly to the patient's heart. Ventricular fibrillation or ventricular tachycardia can be treated, for example, by providing a therapeutic shock to the heart in an attempt to restore normal rhythm.

Such wearable cardiac medical devices can include therapy electrodes that are configured to deliver the therapeutic shocks to a patient. The proper placement and orientation of the therapy electrodes is advantageous to delivering effective cardiac therapy.

### SUMMARY

Disclosed are electrode placement verification systems for cardiac devices including wearable cardiac devices. For example, motion signals can be received from one or more motion sensors integrated or attached to therapy electrodes of the wearable cardiac device. The motion signals can be processed to determine if a corresponding therapy electrodes is properly oriented.

In some aspects, the techniques described herein relate to a wearable cardiac device configured to verify facing orientation of electrodes in a garment worn by an ambulatory patient, the device including: the garment configured to be worn on a torso of the ambulatory patient, the garment including one or more therapy electrode pockets configured to align with one or more predetermined anatomical locations on the ambulatory patient; a plurality of ECG sensing electrodes configured to sense ECG signals of the ambulatory patient, wherein the plurality of ECG sensing electrodes are configured to be disposed at one or more predetermined ECG sensing locations within the garment; a plurality of therapy electrodes configured to deliver one or more therapeutic shocks to the ambulatory patient, wherein each therapy electrode of the plurality of therapy electrodes is configured to be disposed within a therapy electrode pocket of the one or more therapy electrode pockets of the garment; a plurality of anatomical placement accelerometers, wherein each anatomical placement accelerometer is integrated with a corresponding therapy electrode of the plurality of therapy electrodes and configured to generate an anatomical placement motion signal for the corresponding therapy electrode; and a controller in electrical communication with the plurality of electrodes and the plurality of anatomical placement accelerometers, the controller configured to receive anatomical placement motion signals from the plurality of anatomical placement accelerometers, determine, based on the received anatomical placement motion signals, at least one of (a) a facing orientation of the plurality of therapy electrodes, or (b) a relative position of each therapy electrode relative to another therapy electrode of the plurality of therapy electrodes, generate, responsive to a determination that the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment, an improper electrode placement in garment alert for the patient, and provide the generated improper electrode placement in garment alert to the patient.

In some aspects, the techniques described herein relate to a wearable cardiac device, further including one or more physiological sensors separate from the plurality of ECG sensing electrodes and the plurality of therapy electrodes and the plurality of anatomical placement accelerometers, the one or more physiological sensors configured to sense physiological signals from the ambulatory patient.

In some aspects, the techniques described herein relate to a wearable cardiac device, wherein the physiological sensors includes one or more of cardiovibrational sensors for sensing heart sounds, RF antenna and circuitry for determining lung fluid metrics and/or PPG sensors for determining blood oxygenation.

In some aspects, the techniques described herein relate to a wearable cardiac device, wherein the one or more therapy electrodes are configured to deliver one or more therapeutic shocks to the patient responsive to detection of a cardiac arrhythmia based on ECG signals sensed from the plurality of ECG sensing electrodes.

In some aspects, the techniques described herein relate to a wearable cardiac device, wherein each of the one or more therapy electrodes is coupled to a respective ECG sensing electrode of the one or more ECG sensing electrodes to form a multifunctional electrode.

In some aspects, the techniques described herein relate to a wearable cardiac device, wherein the garment includes one or more garment attachment features for removably coupling the plurality of ECG sensing electrodes, the plurality of therapy electrodes and/or the plurality of anatomical placement accelerometers to the garment.

In some aspects, the techniques described herein relate to a wearable cardiac device, wherein the garment attachment features include clasps, hook and loop fasteners, button and hole fasteners, and/or snap buttons.

In some aspects, the techniques described herein relate to a wearable cardiac device, wherein the garment includes one or more garment attachment features for permanently coupling the plurality of ECG sensing electrodes, the plurality of therapy electrodes and/or the plurality of anatomical placement accelerometers to the garment.

In some aspects, the techniques described herein relate to a wearable cardiac device, wherein the garment attachment features include one or more seams for the garment.

In some aspects, the techniques described herein relate to a wearable cardiac device, further including a plurality of anatomical placement gyroscope sensors, wherein each anatomical placement gyroscope sensor is integrated with a corresponding anatomical placement accelerometer and configured to generate the anatomical placement motion signal for the corresponding therapy electrode.

In some aspects, the techniques described herein relate to a wearable cardiac device, wherein the anatomical placement gyroscope sensor is configured to generate at least one anatomical placement motion signal indicating at least one of a rotation direction, rotation angle, and/or vibration.

In some aspects, the techniques described herein relate to a wearable cardiac device, further including a plurality of anatomical placement inertial motion unit sensors, wherein each anatomical placement inertial motion unit sensor is integrated with a corresponding anatomical placement accelerometer and configured to generate the anatomical placement motion signal for the corresponding therapy electrode.

In some aspects, the techniques described herein relate to a wearable cardiac device, wherein the anatomical placement inertial motion unit is configured to generate at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a wearable cardiac device, wherein the anatomical placement accelerometer is configured to generate at least one anatomical placement motion signal indicating linear acceleration in an x-axis, y-axis, and/or z-axis.

In some aspects, the techniques described herein relate to a wearable cardiac device, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: applying a filter to the received at least one anatomical placement motion signal, and/or applying an alignment in time to the received at least one anatomical placement motion signal.

In some aspects, the techniques described herein relate to a wearable cardiac device, wherein determining at least one of the facing orientation of the plurality of therapy electrodes, or the relative position of each therapy electrode relative to another therapy electrode of the plurality of therapy electrodes further includes processing the received at least one anatomical placement motion signal by filtering the anatomical placement motion signal to remove a contribution due to patient breathing from the anatomical placement motion signal.

In some aspects, the techniques described herein relate to a wearable cardiac device, wherein the predetermined electrode placement criterion includes at least one of a prescribed location, prescribed orientation, and/or prescribed facing orientation.

In some aspects, the techniques described herein relate to a wearable cardiac device, wherein the prescribed location is based on a distance from the therapy electrode to a central origin of the ambulatory patient.

In some aspects, the techniques described herein relate to a wearable cardiac device, wherein the prescribed orientation includes an angle formed between a side of the therapy electrode and a central origin of the ambulatory patient.

In some aspects, the techniques described herein relate to a wearable cardiac device, wherein the prescribed facing orientation includes a direction as to which therapeutic shock elements of the therapy electrodes are prescribed to be facing.

In some aspects, the techniques described herein relate to a wearable cardiac device, wherein the predetermined electrode placement criterion is based on historical electrode placement of the at least one of the plurality of therapy electrodes, a predefined electrode placement condition, and/or a population based electrode placement.

In some aspects, the techniques described herein relate to a wearable cardiac device, wherein the facing orientation of the plurality of therapy electrodes includes a direction as to which therapeutic shock elements of the plurality of therapy electrodes are facing.

In some aspects, the techniques described herein relate to a wearable cardiac device, wherein the relative position of each therapy electrode relative to another therapy electrode of the plurality of therapy electrodes includes an inter-therapy electrode distance and inter-therapy electrode angle.

In some aspects, the techniques described herein relate to a wearable cardiac device, the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment includes making a comparison with electrode placements associated with a remainder of the plurality of therapy electrodes.

In some aspects, the techniques described herein relate to a wearable cardiac device wherein the comparison includes at least one of an angle, a direction, or a magnitude.

In some aspects, the techniques described herein relate to a wearable cardiac device, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in an angle of the associated plurality of therapy electrodes and an angle of the predetermined electrode placement criterion is greater than 5% of the angle of the predetermined electrode placement criterion.

In some aspects, the techniques described herein relate to a wearable cardiac device, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a direction of the associated at least one of the plurality of electrodes and a direction of the predetermined electrode placement criterion is greater than 5% of the direction of the predetermined electrode placement criterion.

In some aspects, the techniques described herein relate to a wearable cardiac device, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a magnitude of the associated at least one of the plurality of electrodes and a magnitude of the predetermined electrode placement criterion is greater than 5% of the magnitude of the predetermined electrode placement criterion.

In some aspects, the techniques described herein relate to a device, wherein the relative position of each therapy electrode in the plurality of therapy electrodes includes a treatment vector for sequential shocks being applied to ambulatory patient.

In some aspects, the techniques described herein relate to a wearable cardiac device, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: determining a four-dimensional representation of the plurality of therapy electrodes; and determining a therapy electrode orientation based on the determined four-dimensional representation.

In some aspects, the techniques described herein relate to a wearable cardiac device, wherein the controller is further configured to: identify the at least one therapy electrode among the plurality of therapy electrodes having a corresponding anatomical placement accelerometer integrated with a corresponding therapy electrode of the plurality of therapy electrodes that does not meet the predetermined electrode placement criterion, and disable the identified at least one therapy electrode from delivering one or more therapeutic shocks.

In some aspects, the techniques described herein relate to a wearable cardiac device, wherein the improper electrode placement in garment alert includes an audible indicator, visual indicator, or vibration.

In some aspects, the techniques described herein relate to a wearable cardiac device, further including a graphical user interface in electrical communication with the controller, the graphical user interface further configured to display the improper electrode placement in garment alert.

In some aspects, the techniques described herein relate to a wearable cardiac device, wherein the improper electrode placement in garment alert indicates an identity of an associated at least one of the plurality of therapy electrodes.

In some aspects, the techniques described herein relate to a wearable cardiac device, further including: monitoring the patient for a determining whether a corrective action is taken; and if a corrective action has not been taken, transmitting the generated improper electrode placement in garment alert to a second party device.

In some aspects, the techniques described herein relate to a wearable cardiac device, wherein the controller is further configured to: determine electrode fall-off based at least on the received at least one anatomical placement motion signal.

In some aspects, the techniques described herein relate to a wearable cardiac device configured to verify anatomical placement of electrodes on an ambulatory patient, the device including: a garment configured to be worn on a torso of the ambulatory patient, the garment including one or more garment attachment features aligned with one or more anatomical locations on a patient; a plurality of electrodes configured to one of sense ECG signals of a patient or deliver one or more therapeutic shocks to the patient, wherein each electrode of the plurality of electrodes is configured to be removably coupled to the garment at the one or more anatomical locations on the ambulatory patient via the one or more garment attachment features; at least one anatomical placement sensor circuit associated with at least one of the plurality of electrodes and configured to generate at least one anatomical placement motion signal associated with the at least one of the plurality of electrodes; and a controller in electrical communication with the plurality of electrodes and the at least one anatomical placement sensor circuit, the controller configured to receive the generated at least one anatomical placement motion signal from the at least one anatomical placement sensor circuit; determine, based on the received at least one anatomical placement motion signal, an anatomical placement of the associated at least one of the plurality of electrodes; generate, responsive to a determination that the anatomical placement of the associated at least one of the plurality of electrodes does not meet an anatomical placement condition including a location and orientation of the plurality of electrodes within the garment, an improper electrode placement in garment alert for the patient; and provide the generated improper electrode placement in garment alert to the patient.

In some aspects, the techniques described herein relate to a wearable cardiac device, wherein the garment includes one or more therapy electrode pockets configured to align with the one or more anatomical locations on the patient.

In some aspects, the techniques described herein relate to a wearable cardiac device, wherein the plurality of electrodes includes ECG sensing electrodes configured to sense the ECG signals of the ambulatory patient, wherein the plurality of ECG sensing electrodes are configured to be disposed at one or more predetermined ECG sensing locations within the garment.

In some aspects, the techniques described herein relate to a wearable cardiac device, wherein the plurality of electrodes includes a plurality of therapy electrodes configured to deliver one or more therapeutic shocks to the ambulatory patient, wherein each therapy electrode of the plurality of therapy electrodes is configured to be disposed within a therapy electrode pocket of one or more therapy electrode pockets of the garment.

In some aspects, the techniques described herein relate to a wearable cardiac device, wherein the plurality of electrodes includes ECG sensing electrodes configured to sense the ECG signals of the ambulatory patient and a plurality of therapy electrodes configured to deliver one or more therapeutic shocks to the ambulatory patient.

In some aspects, the techniques described herein relate to a wearable cardiac device further including one or more physiological sensors configured to sense physiological signals from the ambulatory patient.

In some aspects, the techniques described herein relate to a wearable cardiac device wherein the physiological sensors includes one or more of cardiovibrational sensors for sensing heart sounds, RF antenna and circuitry for determining lung fluid metrics and/or PPG sensors for determining blood oxygenation.

In some aspects, the techniques described herein relate to a wearable cardiac device, further including a plurality of therapy electrodes configured to deliver one or more therapeutic shocks to the ambulatory patient responsive to detection of a cardiac arrhythmia based on ECG signals sensed from a plurality of ECG sensing electrodes.

In some aspects, the techniques described herein relate to a wearable cardiac device, wherein the garment attachment features are configured to removably couple the plurality of electrodes and/or the at least one anatomical placement sensor circuit to the garment.

In some aspects, the techniques described herein relate to a wearable cardiac device, wherein the garment attachment features includes include clasps, hook and loop fasteners, button and hole fasteners, and/or snap buttons.

In some aspects, the techniques described herein relate to a wearable cardiac device, wherein the garment attachment feature include one or more elements for permanently coupling the plurality of electrodes and/or the at least one anatomical placement sensor circuit to the garment.

In some aspects, the techniques described herein relate to a wearable cardiac device, wherein the garment attachment features include one or more seams for the garment.

In some aspects, the techniques described herein relate to a wearable cardiac device, wherein the at least one anatomical placement sensor circuit includes at least one of an anatomical placement accelerometer, an anatomical placement gyroscope, and/or an anatomical placement inertial motion unit.

In some aspects, the techniques described herein relate to a wearable cardiac device, wherein the anatomical placement gyroscope sensor is configured to generate at least one anatomical placement motion signal indicating at least one of a rotation direction, rotation angle, and/or vibration.

In some aspects, the techniques described herein relate to a wearable cardiac device, wherein the anatomical placement inertial motion unit is configured to generate at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a wearable cardiac device, wherein the anatomical placement accelerometer is configured to generate at least one anatomical placement motion signal indicating linear acceleration in an x-axis, y-axis, and/or z-axis.

In some aspects, the techniques described herein relate to a wearable cardiac device, wherein the anatomical placement of the associated at least one of the plurality of electrodes includes a facing orientation of the plurality of electrodes.

In some aspects, the techniques described herein relate to a wearable cardiac device, wherein the anatomical placement of the associated at least one of the plurality of electrodes includes a relative position of each electrode relative to another electrode of the plurality of electrodes.

In some aspects, the techniques described herein relate to a wearable cardiac device, wherein the facing orientation includes a direction as to which therapeutic shock elements of the plurality of electrodes are facing.

In some aspects, the techniques described herein relate to a wearable cardiac device, wherein the relative position of each electrode relative to another electrode of the plurality of electrodes includes an inter-therapy electrode distance and an inter-therapy electrode angle.

In some aspects, the techniques described herein relate to a wearable cardiac device, further including: removing a contribution due to patient breathing from the anatomical placement motion signal.

In some aspects, the techniques described herein relate to a wearable cardiac device, wherein determining that the anatomical placement of the associated at least one of the plurality of electrodes does not meet an anatomical placement condition including a location and orientation of the plurality of electrodes within the garment includes a comparison with electrode placements associated with a remainder of the plurality of electrodes.

In some aspects, the techniques described herein relate to a wearable cardiac device wherein the comparison includes at least one of an angle, a direction, or a magnitude.

In some aspects, the techniques described herein relate to a wearable cardiac device, wherein the anatomical placement of the associated at least one of the plurality of electrodes does not meet an anatomical placement condition when a difference in an angle of the associated plurality of electrodes and an angle of the anatomical placement condition is greater than 5% of the angle of the placement condition.

In some aspects, the techniques described herein relate to a wearable cardiac device, wherein the anatomical placement of the associated at least one of the plurality of electrodes does not meet an anatomical placement condition when a difference in a direction of the associated plurality of electrodes and a direction of the anatomical placement condition is greater than 5% of the angle of the placement condition.

In some aspects, the techniques described herein relate to a wearable cardiac device, wherein the anatomical placement of the associated at least one of the plurality of electrodes does not meet an anatomical placement condition when a difference in a magnitude of the associated plurality of electrodes and a direction of the anatomical placement condition is greater than 5% of the magnitude of the placement condition.

In some aspects, the techniques described herein relate to a device, wherein a relative position of each electrode in the plurality of electrodes includes a treatment vector for sequential shocks being applied to ambulatory patient.

In some aspects, the techniques described herein relate to a wearable cardiac device, wherein determining the anatomical placement of the associated at least one of the plurality of electrodes includes: determining a four-dimensional representation of the plurality of electrodes; and determining an electrode orientation based on the determined four-dimensional representation.

In some aspects, the techniques described herein relate to a wearable cardiac device, wherein the controller is further configured to: identify the at least one electrode among the plurality of electrodes having a corresponding anatomical placement that does not meet the anatomical placement condition; and disable the identified at least one electrode from delivering one or more therapeutic shocks.

In some aspects, the techniques described herein relate to a wearable cardiac device, wherein the improper electrode placement in garment alert includes an audible indicator, visual indicator, or vibration.

In some aspects, the techniques described herein relate to a wearable cardiac device, further including a graphical user interface in electrical communication with the controller, the graphical user interface further configured to display the improper electrode placement in garment alert.

In some aspects, the techniques described herein relate to a wearable cardiac device, wherein the improper electrode placement in garment alert indicates an identity of the associated at least one of the plurality of electrodes.

In some aspects, the techniques described herein relate to a wearable cardiac device, further including: monitoring the patient for a determining whether a corrective action is taken; and if a corrective action has not been taken, transmitting the generated improper electrode placement in garment alert to a second party device.

In some aspects, the techniques described herein relate to a wearable cardiac device, wherein the controller is further configured to: determine electrode fall-off based at least on the received at least one anatomical placement motion signal.

In some aspects, the techniques described herein relate to a method for electrode placement verification in a garment configured to be worn on a torso of an ambulatory patient, the garment including one or more therapy electrode pockets configured to align with one or more predetermined anatomical locations on the ambulatory patient, the method including: receiving anatomical placement motion signals from a plurality of anatomical placement accelerometers, wherein each anatomical placement accelerometer of the plurality of anatomical placement accelerometers is integrated with a corresponding therapy electrode of a plurality of therapy electrodes and configured to generate an anatomical placement motion signal for the corresponding therapy electrode, wherein the plurality of therapy electrodes are configured to deliver one or more therapeutic shocks to the ambulatory patient, and wherein each therapy electrode of the plurality of therapy electrodes is configured to be disposed within the therapy electrode pocket of the one or more therapy electrode pockets of the garment; determining, based on the received anatomical placement motion signals, at least one of (c) a facing orientation of the plurality of therapy electrodes, or (d) a relative position of each therapy electrode relative to another therapy electrode of the plurality of therapy electrodes; generating, responsive to determining that the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment, an improper electrode placement in garment alert for the patient; and providing the generated improper electrode placement in garment alert to the patient.

In some aspects, the techniques described herein relate to a method, further including: sensing physiological signals from the ambulatory patient via one or more physiological sensors separate from the plurality of therapy electrodes and the plurality of anatomical placement accelerometers.

In some aspects, the techniques described herein relate to a method, wherein the physiological sensors includes one or more of cardiovibrational sensors for sensing heart sounds, RF antenna and circuitry for determining lung fluid metrics and/or PPG sensors for determining blood oxygenation.

In some aspects, the techniques described herein relate to a method, further including: delivering one or more therapeutic shocks to the patient via the one or more therapy electrodes responsive to detecting a cardiac arrhythmia based on ECG signals sensed from a plurality of ECG sensing electrodes.

In some aspects, the techniques described herein relate to a method, wherein each of the one or more therapy electrodes is coupled to a respective ECG sensing electrode of one or more ECG sensing electrodes to form a multifunctional electrode.

In some aspects, the techniques described herein relate to a method, wherein the garment includes one or more garment attachment features for removably coupling a plurality of ECG sensing electrodes, the plurality of therapy electrodes and/or the plurality of anatomical placement accelerometers to the garment.

In some aspects, the techniques described herein relate to a method, wherein the garment attachment features include clasps, hook and loop fasteners, button and hole fasteners, and/or snap buttons.

In some aspects, the techniques described herein relate to a method, wherein the garment includes one or more garment attachment features for permanently coupling a plurality of ECG sensing electrodes, the plurality of therapy electrodes and/or the plurality of anatomical placement accelerometers to the garment.

In some aspects, the techniques described herein relate to a method, wherein the garment attachment features include one or more seams for the garment.

In some aspects, the techniques described herein relate to a method, further including: generating the anatomical placement motion signal via a plurality of anatomical placement gyroscope sensors, wherein each anatomical placement gyroscope sensor is integrated with a corresponding anatomical placement accelerometer and configured to generate the anatomical placement motion signal for the corresponding therapy electrode.

In some aspects, the techniques described herein relate to a method, wherein the anatomical placement gyroscope sensor is configured to generate at least one anatomical placement motion signal indicating at least one of a rotation direction, rotation angle, and/or vibration.

In some aspects, the techniques described herein relate to a method, further including a plurality of anatomical placement inertial motion unit sensors, wherein each anatomical placement inertial motion unit sensor is integrated with a corresponding anatomical placement accelerometer and configured to generate the anatomical placement motion signal for the corresponding therapy electrode.

In some aspects, the techniques described herein relate to a method, further including: generating, via the anatomical placement inertial motion unit, at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a method, further including: generating, via the anatomical placement accelerometer, at least one anatomical placement motion signal indicating linear acceleration in an x-axis, y-axis, and/or z-axis.

In some aspects, the techniques described herein relate to a method, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: applying a filter to the received at least one anatomical placement motion signal, and/or applying an alignment in time to the received at least one anatomical placement motion signal.

In some aspects, the techniques described herein relate to a method, wherein determining at least one of the facing orientation of the plurality of therapy electrodes, or the relative position of each therapy electrode relative to another therapy electrode of the plurality of therapy electrodes further includes processing the received at least one anatomical placement motion signal by filtering the anatomical placement motion signal to remove a contribution due to patient breathing from the anatomical placement motion signal.

In some aspects, the techniques described herein relate to a method, wherein the predetermined electrode placement criterion includes at least one of a prescribed location, prescribed orientation, and/or prescribed facing orientation.

In some aspects, the techniques described herein relate to a method, wherein the prescribed location is based on a distance from the therapy electrode to a central origin of the ambulatory patient.

In some aspects, the techniques described herein relate to a method, wherein the prescribed orientation includes an angle formed between a side of the therapy electrode and a central origin of the ambulatory patient.

In some aspects, the techniques described herein relate to a method, wherein the prescribed facing orientation includes a direction as to which therapeutic shock elements of the therapy electrodes are prescribed to be facing.

In some aspects, the techniques described herein relate to a method, wherein the predetermined electrode placement criterion is based on historical electrode placement of the at least one of the plurality of therapy electrodes, a predefined electrode placement condition, and/or a population based electrode placement.

In some aspects, the techniques described herein relate to a method, wherein the facing orientation of the plurality of therapy electrodes includes a direction as to which therapeutic shock elements of the plurality of therapy electrodes are facing.

In some aspects, the techniques described herein relate to a method, wherein the relative position of each therapy electrode relative to another therapy electrode of the plurality of therapy electrodes includes an inter-therapy electrode distance and inter-therapy electrode angle.

In some aspects, the techniques described herein relate to a method, the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment includes making a comparison with electrode placements associated with a remainder of the plurality of therapy electrodes.

In some aspects, the techniques described herein relate to a method wherein the comparison includes at least one of an angle, a direction, or a magnitude.

In some aspects, the techniques described herein relate to a method, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in an angle of the associated plurality of therapy electrodes and an angle of the predetermined electrode placement criterion is greater than 5% of the angle of the predetermined electrode placement criterion.

In some aspects, the techniques described herein relate to a method, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a direction of the associated at least one of the plurality of electrodes and a direction of the predetermined electrode placement criterion is greater than 5% of the direction of the predetermined electrode placement criterion.

In some aspects, the techniques described herein relate to a method, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a magnitude of the associated at least one of the plurality of electrodes and a magnitude of the predetermined electrode placement criterion is greater than 5% of the magnitude of the predetermined electrode placement criterion.

In some aspects, the techniques described herein relate to a method, wherein the relative position of each therapy electrode in the plurality of therapy electrodes includes a treatment vector for sequential shocks being applied to ambulatory patient.

In some aspects, the techniques described herein relate to a method, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: determining a four-dimensional representation of the plurality of therapy electrodes; and determining a therapy electrode orientation based on the determined four-dimensional representation.

In some aspects, the techniques described herein relate to a method, further including: identifying the at least one therapy electrode among the plurality of therapy electrodes having a corresponding anatomical placement accelerometer integrated with a corresponding therapy electrode of the plurality of therapy electrodes that does not meet the predetermined electrode placement criterion, and disabling the identified at least one therapy electrode from delivering one or more therapeutic shocks.

In some aspects, the techniques described herein relate to a method, wherein the improper electrode placement in garment alert includes an audible indicator, visual indicator, or vibration.

In some aspects, the techniques described herein relate to a method, further including: displaying the improper electrode placement in garment alert in a graphical user interface.

In some aspects, the techniques described herein relate to a method, wherein the improper electrode placement in garment alert indicates an identity of an associated at least one of the plurality of therapy electrodes.

In some aspects, the techniques described herein relate to a method, further including: monitoring the patient for a determining whether a corrective action is taken; and if a corrective action has not been taken, transmitting the generated improper electrode placement in garment alert to a second party device.

In some aspects, the techniques described herein relate to a method, further including: determining electrode fall-off based at least on the received at least one anatomical placement motion signal.

In some aspects, the techniques described herein relate to a method for placement verification of electrodes on an ambulatory patient, the method including: receiving at least one anatomical placement motion signal from at least one anatomical placement sensor circuit, wherein the at least one anatomical placement sensor circuit is associated with at least one of a plurality of electrodes and configured to generate at least one anatomical placement motion signal associated with the at least one of the plurality of electrodes, the plurality of electrodes configured to one of sense ECG signals of the ambulatory patient or deliver one or more therapeutic shocks to the ambulatory patient, wherein each electrode of the plurality of electrodes is configured to be removably coupled to a garment configured to be worn on a torso of the ambulatory patient, the garment including one or more garment attachment features aligned with one or more anatomical locations on the ambulatory patient at the one or more anatomical locations on the ambulatory patient via the one or more garment attachment features; determining, based on the received at least one anatomical placement motion signal, an anatomical placement of the associated at least one of the plurality of electrodes; generating, responsive to determining that the anatomical placement of the associated at least one of the plurality of electrodes does not meet an anatomical placement condition including a location and orientation of the plurality of electrodes within the garment, an improper electrode placement in garment alert for the patient; and providing the generated improper electrode placement in garment alert to the patient.

In some aspects, the techniques described herein relate to a method, wherein the garment includes one or more therapy electrode pockets configured to align with the one or more anatomical locations on the patient.

In some aspects, the techniques described herein relate to a method, further including: sensing ECG signals of the ambulatory patient, wherein the plurality of electrodes includes ECG sensing electrodes configured to sense the ECG signals of the ambulatory patient, and the plurality of ECG sensing electrodes are configured to be disposed at one or more predetermined ECG sensing locations within the garment.

In some aspects, the techniques described herein relate to a method, further including: delivering one or more therapeutic shocks to the ambulatory patient via a plurality of therapy electrodes, wherein the plurality of electrodes includes the plurality of therapy electrodes, and wherein each therapy electrode of the plurality of therapy electrodes is configured to be disposed within a therapy electrode pocket of one or more therapy electrode pockets of the garment.

In some aspects, the techniques described herein relate to a method, further including: sensing ECG signals of the ambulatory patient via ECG sensing electrodes, wherein the plurality of electrodes includes ECG sensing electrodes configured to sense the ECG signals of the ambulatory patient and a plurality of therapy electrodes configured to deliver one or more therapeutic shocks to the ambulatory patient.

In some aspects, the techniques described herein relate to a method further including: sensing physiological signals from the ambulatory patient via one or more physiological sensors.

In some aspects, the techniques described herein relate to a method wherein the physiological sensors include one or more of cardiovibrational sensors for sensing heart sounds, RF antenna and circuitry for determining lung fluid metrics and/or PPG sensors for determining blood oxygenation.

In some aspects, the techniques described herein relate to a method, further including: delivering, via a plurality of therapy electrodes one or more therapeutic shocks to the ambulatory patient, responsive to detection of a cardiac arrhythmia based on ECG signals sensed from a plurality of ECG sensing electrodes.

In some aspects, the techniques described herein relate to a method, wherein the garment attachment features are configured to removably couple the plurality of electrodes and/or the at least one anatomical placement sensor circuit to the garment.

In some aspects, the techniques described herein relate to a method, wherein the garment attachment features includes include clasps, hook and loop fasteners, button and hole fasteners, and/or snap buttons.

In some aspects, the techniques described herein relate to a method, wherein the garment attachment feature include one or more elements for permanently coupling the plurality of electrodes and/or the at least one anatomical placement sensor circuit to the garment.

In some aspects, the techniques described herein relate to a method, wherein the garment attachment features include one or more seams for the garment.

In some aspects, the techniques described herein relate to a method, wherein the at least one anatomical placement sensor circuit includes at least one of an anatomical placement accelerometer, an anatomical placement gyroscope, and/or an anatomical placement inertial motion unit.

In some aspects, the techniques described herein relate to a method, wherein the anatomical placement gyroscope sensor is configured to generate at least one anatomical placement motion signal indicating at least one of a rotation direction, rotation angle, and/or vibration.

In some aspects, the techniques described herein relate to a method, further including generating at least one anatomical placement motion signal via the anatomical placement inertial motion unit, wherein the at least one anatomical placement motion signal indicates at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a method, further including: generating at least one anatomical placement motion signal via the anatomical placement accelerometer, wherein the anatomical placement motion signal is configured to indicate linear acceleration in an x-axis, y-axis, and/or z-axis.

In some aspects, the techniques described herein relate to a method, wherein the anatomical placement of the associated at least one of the plurality of electrodes includes a facing orientation of the plurality of electrodes.

In some aspects, the techniques described herein relate to a method, wherein the anatomical placement of the associated at least one of the plurality of electrodes includes a relative position of each electrode relative to another electrode of the plurality of electrodes.

In some aspects, the techniques described herein relate to a method, wherein the facing orientation includes a direction as to which therapeutic shock elements of the plurality of electrodes are facing.

In some aspects, the techniques described herein relate to a method, wherein the relative position of each electrode relative to another electrode of the plurality of electrodes includes an inter-therapy electrode distance and an inter-therapy electrode angle.

In some aspects, the techniques described herein relate to a method, further including: removing a contribution due to patient breathing from the anatomical placement motion signal.

In some aspects, the techniques described herein relate to a method, wherein determining that the anatomical placement of the associated at least one of the plurality of electrodes does not meet an anatomical placement condition including a location and orientation of the plurality of electrodes within the garment includes a comparison with electrode placements associated with a remainder of the plurality of electrodes.

In some aspects, the techniques described herein relate to a method wherein the comparison includes at least one of an angle, a direction, or a magnitude.

In some aspects, the techniques described herein relate to a method, wherein the anatomical placement of the associated at least one of the plurality of electrodes does not meet an anatomical placement condition when a difference in an angle of the associated plurality of electrodes and an angle of the anatomical placement condition is greater than 5% of the angle of the placement condition.

In some aspects, the techniques described herein relate to a method, wherein the anatomical placement of the associated at least one of the plurality of electrodes does not meet an anatomical placement condition when a difference in a direction of the associated plurality of electrodes and a direction of the anatomical placement condition is greater than 5% of the angle of the placement condition.

In some aspects, the techniques described herein relate to a method, wherein the anatomical placement of the associated at least one of the plurality of electrodes does not meet an anatomical placement condition when a difference in a magnitude of the associated plurality of electrodes and a direction of the anatomical placement condition is greater than 5% of the magnitude of the placement condition.

In some aspects, the techniques described herein relate to a method, wherein a relative position of each electrode in the plurality of electrodes includes a treatment vector for sequential shocks being applied to ambulatory patient.

In some aspects, the techniques described herein relate to a method, wherein determining the anatomical placement of the associated at least one of the plurality of electrodes includes: determining a four-dimensional representation of the plurality of electrodes; and determining an electrode orientation based on the determined four-dimensional representation.

In some aspects, the techniques described herein relate to a method, further including: identifying the at least one electrode among the plurality of electrodes having a corresponding anatomical placement that does not meet the anatomical placement condition; and disabling the identified at least one electrode from delivering one or more therapeutic shocks.

In some aspects, the techniques described herein relate to a method, wherein the improper electrode placement in garment alert includes an audible indicator, visual indicator, or vibration.

In some aspects, the techniques described herein relate to a method, further including: displaying the improper electrode placement in garment alert in a graphical user interface.

In some aspects, the techniques described herein relate to a method, wherein the improper electrode placement in garment alert indicates an identity of the associated at least one of the plurality of electrodes.

In some aspects, the techniques described herein relate to a method, further including: monitoring the patient for a determining whether a corrective action is taken; and if a corrective action has not been taken, transmitting the generated improper electrode placement in garment alert to a second party device.

In some aspects, the techniques described herein relate to a method, further including: determining an electrode fall-off based at least on the received at least one anatomical placement motion signal.

In some aspects, the techniques described herein relate to a non-transitory computer program product storing instructions, which when executed by at least one processor of at least one computing system, cause the at least one processor to perform operations for electrode placement verification in a garment configured to be worn on a torso of an ambulatory patient, the garment including one or more therapy electrode pockets configured to align with one or more predetermined anatomical locations on the ambulatory patient, the operations including: receiving anatomical placement motion signals from a plurality of anatomical placement accelerometers, wherein each anatomical placement accelerometer of the plurality of anatomical placement accelerometers is integrated with a corresponding therapy electrode of a plurality of therapy electrodes and configured to generate an anatomical placement motion signal for the corresponding therapy electrode, wherein the plurality of therapy electrodes are configured to deliver one or more therapeutic shocks to the ambulatory patient, and wherein each therapy electrode of the plurality of therapy electrodes is configured to be disposed within the therapy electrode pocket of the one or more therapy electrode pockets of the garment; determining, based on the received anatomical placement motion signals, at least one of (e) a facing orientation of the plurality of therapy electrodes, or (f) a relative position of each therapy electrode relative to another therapy electrode of the plurality of therapy electrodes; generating, responsive to determining that the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment, an improper electrode placement in garment alert for the patient; and providing the generated improper electrode placement in garment alert to the patient.

In some aspects, the techniques described herein relate to a non-transitory computer program product, the operations further including: sensing physiological signals from the ambulatory patient via one or more physiological sensors separate from the plurality of therapy electrodes and the plurality of anatomical placement accelerometers.

In some aspects, the techniques described herein relate to a non-transitory computer program product, wherein the physiological sensors includes one or more of cardiovibrational sensors for sensing heart sounds, RF antenna and circuitry for determining lung fluid metrics and/or PPG sensors for determining blood oxygenation.

In some aspects, the techniques described herein relate to a non-transitory computer program product, the operations further including: delivering one or more therapeutic shocks to the patient via the one or more therapy electrodes responsive to detecting a cardiac arrhythmia based on ECG signals sensed from a plurality of ECG sensing electrodes.

In some aspects, the techniques described herein relate to a non-transitory computer program product, wherein each of the one or more therapy electrodes is coupled to a respective ECG sensing electrode of one or more ECG sensing electrodes to form a multifunctional electrode.

In some aspects, the techniques described herein relate to a non-transitory computer program product, wherein the garment includes one or more garment attachment features for removably coupling a plurality of ECG sensing electrodes, the plurality of therapy electrodes and/or the plurality of anatomical placement accelerometers to the garment.

In some aspects, the techniques described herein relate to a non-transitory computer program product, wherein the garment attachment features include clasps, hook and loop fasteners, button and hole fasteners, and/or snap buttons.

In some aspects, the techniques described herein relate to a non-transitory computer program product, wherein the garment includes one or more garment attachment features for permanently coupling a plurality of ECG sensing electrodes, the plurality of therapy electrodes and/or the plurality of anatomical placement accelerometers to the garment.

In some aspects, the techniques described herein relate to a non-transitory computer program product, wherein the garment attachment features include one or more seams for the garment.

In some aspects, the techniques described herein relate to a non-transitory computer program product, the operations further including: generating the anatomical placement motion signal via a plurality of anatomical placement gyroscope sensors, wherein each anatomical placement gyroscope sensor is integrated with a corresponding anatomical placement accelerometer and configured to generate the anatomical placement motion signal for the corresponding therapy electrode.

In some aspects, the techniques described herein relate to a non-transitory computer program product, wherein the anatomical placement gyroscope sensor is configured to generate at least one anatomical placement motion signal indicating at least one of a rotation direction, rotation angle, and/or vibration.

In some aspects, the techniques described herein relate to a non-transitory computer program product, further including a plurality of anatomical placement inertial motion unit sensors, wherein each anatomical placement inertial motion unit sensor is integrated with a corresponding anatomical placement accelerometer and configured to generate the anatomical placement motion signal for the corresponding therapy electrode.

In some aspects, the techniques described herein relate to a non-transitory computer program product, the operations further including: generating, via the anatomical placement inertial motion unit, at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a non-transitory computer program product, the operations further including: generating, via the anatomical placement accelerometer, at least one anatomical placement motion signal indicating linear acceleration in an x-axis, y-axis, and/or z-axis.

In some aspects, the techniques described herein relate to a non-transitory computer program product, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: applying a filter to the received at least one anatomical placement motion signal, and/or applying an alignment in time to the received at least one anatomical placement motion signal.

In some aspects, the techniques described herein relate to a non-transitory computer program product, wherein determining at least one of the facing orientation of the plurality of therapy electrodes, or the relative position of each therapy electrode relative to another therapy electrode of the plurality of therapy electrodes further includes processing the received at least one anatomical placement motion signal by filtering the anatomical placement motion signal to remove a contribution due to patient breathing from the anatomical placement motion signal.

In some aspects, the techniques described herein relate to a non-transitory computer program product, wherein the predetermined electrode placement criterion includes at least one of a prescribed location, prescribed orientation, and/or prescribed facing orientation.

In some aspects, the techniques described herein relate to a non-transitory computer program product, wherein the prescribed location is based on a distance from the therapy electrode to a central origin of the ambulatory patient.

In some aspects, the techniques described herein relate to a non-transitory computer program product, wherein the prescribed orientation includes an angle formed between a side of the therapy electrode and a central origin of the ambulatory patient.

In some aspects, the techniques described herein relate to a non-transitory computer program product, wherein the prescribed facing orientation includes a direction as to which therapeutic shock elements of the therapy electrodes are prescribed to be facing.

In some aspects, the techniques described herein relate to a non-transitory computer program product, wherein the predetermined electrode placement criterion is based on historical electrode placement of the at least one of the plurality of therapy electrodes, a predefined electrode placement condition, and/or a population based electrode placement.

In some aspects, the techniques described herein relate to a non-transitory computer program product, wherein the facing orientation of the plurality of therapy electrodes includes a direction as to which therapeutic shock elements of the plurality of therapy electrodes are facing.

In some aspects, the techniques described herein relate to a non-transitory computer program product, wherein the relative position of each therapy electrode relative to another therapy electrode of the plurality of therapy electrodes includes an inter-therapy electrode distance and inter-therapy electrode angle.

In some aspects, the techniques described herein relate to a non-transitory computer program product, the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment includes making a comparison with electrode placements associated with a remainder of the plurality of therapy electrodes.

In some aspects, the techniques described herein relate to a non-transitory computer program product wherein the comparison includes at least one of an angle, a direction, or a magnitude.

In some aspects, the techniques described herein relate to a non-transitory computer program product, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in an angle of the associated plurality of therapy electrodes and an angle of the predetermined electrode placement criterion is greater than 5% of the angle of the predetermined electrode placement criterion.

In some aspects, the techniques described herein relate to a non-transitory computer program product, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a direction of the associated at least one of the plurality of electrodes and a direction of the predetermined electrode placement criterion is greater than 5% of the direction of the predetermined electrode placement criterion.

In some aspects, the techniques described herein relate to a non-transitory computer program product, wherein the at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a magnitude of the associated at least one of the plurality of electrodes and a magnitude of the predetermined electrode placement criterion is greater than 5% of the magnitude of the predetermined electrode placement criterion.

In some aspects, the techniques described herein relate to a non-transitory computer program product, wherein the relative position of each therapy electrode in the plurality of therapy electrodes includes a treatment vector for sequential shocks being applied to ambulatory patient.

In some aspects, the techniques described herein relate to a non-transitory computer program product, wherein determining at least one of the facing orientation of the plurality of therapy electrodes, or the relative position of each therapy electrode relative to another therapy electrode of the plurality of therapy electrodes further includes processing the received at least one anatomical placement motion signal by at least one of: determining a four-dimensional representation of the plurality of therapy electrodes; and determining a therapy electrode orientation based on the determined four-dimensional representation.

In some aspects, the techniques described herein relate to a non-transitory computer program product, the operations further including: identifying the at least one therapy electrode among the plurality of therapy electrodes having a corresponding anatomical placement accelerometer integrated with a corresponding therapy electrode of the plurality of therapy electrodes that does not meet the predetermined electrode placement criterion, and disabling the identified at least one therapy electrode from delivering one or more therapeutic shocks.

In some aspects, the techniques described herein relate to a non-transitory computer program product, wherein the improper electrode placement in garment alert includes an audible indicator, visual indicator, or vibration.

In some aspects, the techniques described herein relate to a non-transitory computer program product, the operations further including: displaying the improper electrode placement in garment alert in a graphical user interface.

In some aspects, the techniques described herein relate to a non-transitory computer program product, wherein the improper electrode placement in garment alert indicates an identity of an associated at least one of the plurality of therapy electrodes.

In some aspects, the techniques described herein relate to a non-transitory computer program product, further including: monitoring the patient for a determining whether a corrective action is taken; and if a corrective action has not been taken, transmitting the generated improper electrode placement in garment alert to a second party device.

In some aspects, the techniques described herein relate to a non-transitory computer program product, further including: determining electrode fall-off based at least on the received at least one anatomical placement motion signal.

In some aspects, the techniques described herein relate to a non-transitory computer program product storing instructions, which when executed by at least one processor of at least one computing system, cause the at least one processor to perform operations for placement verification of electrodes on an ambulatory patient, the operations including: receiving at least one anatomical placement motion signal from at least one anatomical placement sensor circuit, wherein the at least one anatomical placement sensor circuit is associated with at least one of a plurality of electrodes and configured to generate at least one anatomical placement motion signal associated with the at least one of the plurality of electrodes, the plurality of electrodes configured to one of sense ECG signals of the ambulatory patient or deliver one or more therapeutic shocks to the ambulatory patient, wherein each electrode of the plurality of electrodes is configured to be removably coupled to a garment configured to be worn on a torso of the ambulatory patient, the garment including one or more garment attachment features aligned with one or more anatomical locations on the ambulatory patient at the one or more anatomical locations on the ambulatory patient via the one or more garment attachment features; determining, based on the received at least one anatomical placement motion signal, an anatomical placement of the associated at least one of the plurality of electrodes; generating, responsive to determining that the anatomical placement of the associated at least one of the plurality of electrodes does not meet an anatomical placement condition including a location and orientation of the plurality of electrodes within the garment, an improper electrode placement in garment alert for the patient; and providing the generated improper electrode placement in garment alert to the patient.

In some aspects, the techniques described herein relate to a non-transitory computer program product, wherein the garment includes one or more therapy electrode pockets configured to align with the one or more anatomical locations on the patient.

In some aspects, the techniques described herein relate to a non-transitory computer program product, the operations further including: sensing ECG signals of the ambulatory patient, wherein the plurality of electrodes includes ECG sensing electrodes configured to sense the ECG signals of the ambulatory patient, and the plurality of ECG sensing electrodes are configured to be disposed at one or more predetermined ECG sensing locations within the garment.

In some aspects, the techniques described herein relate to a non-transitory computer program product, the operations further including: delivering one or more therapeutic shocks to the ambulatory patient via a plurality of therapy electrodes, wherein the plurality of electrodes includes the plurality of therapy electrodes, and wherein each therapy electrode of the plurality of therapy electrodes is configured to be disposed within a therapy electrode pocket of one or more therapy electrode pockets of the garment.

In some aspects, the techniques described herein relate to a non-transitory computer program product, the operations further including: sensing ECG signals of the ambulatory patient via ECG sensing electrodes, wherein the plurality of electrodes includes ECG sensing electrodes configured to sense the ECG signals of the ambulatory patient and a plurality of therapy electrodes configured to deliver one or more therapeutic shocks to the ambulatory patient.

In some aspects, the techniques described herein relate to a non-transitory computer program product, the operations further including: sensing physiological signals from the ambulatory patient via one or more physiological sensors.

In some aspects, the techniques described herein relate to a non-transitory computer program product wherein the physiological sensors include one or more of cardiovibrational sensors for sensing heart sounds, RF antenna and circuitry for determining lung fluid metrics and/or PPG sensors for determining blood oxygenation.

In some aspects, the techniques described herein relate to a non-transitory computer program product, the operations further including: delivering, via a plurality of therapy electrodes one or more therapeutic shocks to the ambulatory patient, responsive to detection of a cardiac arrhythmia based on ECG signals sensed from a plurality of ECG sensing electrodes.

In some aspects, the techniques described herein relate to a non-transitory computer program product, wherein the garment attachment features are configured to removably couple the plurality of electrodes and/or the at least one anatomical placement sensor circuit to the garment.

In some aspects, the techniques described herein relate to a non-transitory computer program product, wherein the garment attachment features includes include clasps, hook and loop fasteners, button and hole fasteners, and/or snap buttons.

In some aspects, the techniques described herein relate to a non-transitory computer program product, wherein the garment attachment feature include one or more elements for permanently coupling the plurality of electrodes and/or the at least one anatomical placement sensor circuit to the garment.

In some aspects, the techniques described herein relate to a non-transitory computer program product, wherein the garment attachment features include one or more seams for the garment.

In some aspects, the techniques described herein relate to a non-transitory computer program product, wherein the at least one anatomical placement sensor circuit includes at least one of an anatomical placement accelerometer, an anatomical placement gyroscope, and/or an anatomical placement inertial motion unit.

In some aspects, the techniques described herein relate to a non-transitory computer program product, wherein the anatomical placement gyroscope sensor is configured to generate at least one anatomical placement motion signal indicating at least one of a rotation direction, rotation angle, and/or vibration.

In some aspects, the techniques described herein relate to a non-transitory computer program product, the operations further including: generating at least one anatomical placement motion signal via the anatomical placement inertial motion unit, wherein the at least one anatomical placement motion signal indicates at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

In some aspects, the techniques described herein relate to a non-transitory computer program product, the operations further including: generating at least one anatomical placement motion signal via the anatomical placement accelerometer, wherein the anatomical placement motion signal is configured to indicate linear acceleration in an x-axis, y-axis, and/or z-axis.

In some aspects, the techniques described herein relate to a non-transitory computer program product, wherein the anatomical placement of the associated at least one of the plurality of electrodes includes a facing orientation of the plurality of electrodes.

In some aspects, the techniques described herein relate to a non-transitory computer program product, wherein the anatomical placement of the associated at least one of the plurality of electrodes includes a relative position of each electrode relative to another electrode of the plurality of electrodes.

In some aspects, the techniques described herein relate to a non-transitory computer program product, wherein the facing orientation includes a direction as to which therapeutic shock elements of the plurality of electrodes are facing.

In some aspects, the techniques described herein relate to a non-transitory computer program product, wherein the relative position of each electrode relative to another electrode of the plurality of electrodes includes an inter-therapy electrode distance and an inter-therapy electrode angle.

In some aspects, the techniques described herein relate to a non-transitory computer program product, further including: removing a contribution due to patient breathing from the anatomical placement motion signal.

In some aspects, the techniques described herein relate to a non-transitory computer program product, wherein determining that the anatomical placement of the associated at least one of the plurality of electrodes does not meet an anatomical placement condition including a location and orientation of the plurality of electrodes within the garment includes a comparison with electrode placements associated with a remainder of the plurality of electrodes.

In some aspects, the techniques described herein relate to a non-transitory computer program product wherein the comparison includes at least one of an angle, a direction, or a magnitude.

In some aspects, the techniques described herein relate to a non-transitory computer program product, wherein the anatomical placement of the associated at least one of the plurality of electrodes does not meet an anatomical placement condition when a difference in an angle of the associated plurality of electrodes and an angle of the anatomical placement condition is greater than 5% of the angle of the placement condition.

In some aspects, the techniques described herein relate to a non-transitory computer program product, wherein the anatomical placement of the associated at least one of the plurality of electrodes does not meet an anatomical placement condition when a difference in a direction of the associated plurality of electrodes and a direction of the anatomical placement condition is greater than 5% of the angle of the placement condition.

In some aspects, the techniques described herein relate to a non-transitory computer program product, wherein the anatomical placement of the associated at least one of the plurality of electrodes does not meet an anatomical placement condition when a difference in a magnitude of the associated plurality of electrodes and a direction of the anatomical placement condition is greater than 5% of the magnitude of the placement condition.

In some aspects, the techniques described herein relate to a non-transitory computer program product, wherein a relative position of each electrode in the plurality of electrodes includes a treatment vector for sequential shocks being applied to ambulatory patient.

In some aspects, the techniques described herein relate to a non-transitory computer program product, wherein determining the anatomical placement of the associated at least one of the plurality of electrodes includes: determining a four-dimensional representation of the plurality of electrodes; and determining an electrode orientation based on the determined four-dimensional representation.

In some aspects, the techniques described herein relate to a non-transitory computer program product, the operations further including: identifying the at least one electrode among the plurality of electrodes having a corresponding anatomical placement that does not meet the anatomical placement condition; and disabling the identified at least one electrode from delivering one or more therapeutic shocks.

In some aspects, the techniques described herein relate to a non-transitory computer program product, wherein the improper electrode placement in garment alert includes an audible indicator, visual indicator, or vibration.

In some aspects, the techniques described herein relate to a non-transitory computer program product, the operations further including: displaying the improper electrode placement in garment alert in a graphical user interface.

In some aspects, the techniques described herein relate to a non-transitory computer program product, wherein the improper electrode placement in garment alert indicates an identity of the associated at least one of the plurality of electrodes.

In some aspects, the techniques described herein relate to a non-transitory computer program product, the operations further including: monitoring the patient for a determining whether a corrective action is taken; and if a corrective action has not been taken, transmitting the generated improper electrode placement in garment alert to a second party device.

In some aspects, the techniques described herein relate to a non-transitory computer program product, the operations further including: determining an electrode fall-off based at least on the received at least one anatomical placement motion signal.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a diagrammatic representation of treatment device positioning on a subject in accordance with some embodiments of the present disclosure;

FIG. 2 is a block diagram of treatment device, in accordance with some embodiments of the present disclosure;

FIG. 3 is a block diagram of a treatment device, in accordance with some embodiments of the present disclosure;

FIG. 4 is an alternative block diagram of a treatment device, in accordance with some embodiments of the present disclosure;

FIG. 5 is a medical monitoring and treatment device, in accordance with some embodiments of the present disclosure;

FIG. 6 is a schematic example of a wearable cardiac device that includes electrode verification components configured for verifying the placement and orientation of therapy electrodes on an ambulatory patient, in accordance with some embodiments of the present disclosure;

FIG. 7 is a second schematic example of a wearable cardiac device that includes electrode placement verification components configured for verifying the placement and orientation of therapy electrodes on an ambulatory patient, in accordance with some embodiments of the present disclosure;

FIG. 8 is a schematic diagram for a controller for a wearable cardiac device in accordance with some embodiments of the present disclosure;

FIG. 9 is a second schematic diagram of a controller for a wearable cardiac device in accordance with some embodiments of the present disclosure;

FIGS. 10A and 10B illustrate schematic diagrams for an alarm module for a wearable cardiac device in accordance with some embodiments of the present disclosure;

FIG. 11 provides an illustration for a first garment style for a wearable cardiac device in accordance with some embodiments of the present disclosure;

FIGS. 12A-12H are an assembly process for an embodiment of a cardiac device including a garment and electrode belt in accordance with some embodiments of the present disclosure;

FIGS. 13A-13G are an assembly process for another embodiment of a wearable cardiac device including a garment and electrode belt in accordance with some embodiments of the present disclosure;

FIG. 14 is a schematic example of a medical device controller for a wearable cardiac device in accordance with some embodiments of the present disclosure;

FIG. 15 is a data transmission system for use in connection with the cardiac wearable device in accordance with some embodiments of the present disclosure;

FIG. 16 is a process or method for verifying facing orientation of electrodes such as treatment or monitoring electrodes in a garment worn by an ambulatory patient in accordance with some embodiments of the present disclosure;

FIG. 17 is a process or method for verifying anatomical placement of electrodes on an ambulatory patient in accordance with some embodiments of the present disclosure;

FIGS. 18A-18C illustrate motion data for an ambulatory subject in accordance with some embodiments of the present disclosure;

FIG. 19 is the expected anatomical placement motion signals for therapy electrodes of a wearable cardiac device in accordance with some embodiments of the present disclosure;

FIG. 20 is a graphical illustration of processed data from anatomical placement motion signals for a wearable cardiac device in accordance with some embodiments of the present disclosure;

FIG. 21 is a graphical illustration of processed data from anatomical placement motion signals for a wearable cardiac device in accordance with some embodiments of the present disclosure;

FIG. 22A is a first view of a therapy electrode rendered by a visualization process in accordance with some embodiments of the present disclosure;

FIG. 22B is a side view of a therapy electrode rendered by a visualization process in accordance with some embodiments of the present disclosure;

FIG. 23 is a schematic for inertial motion units in communication with therapy electrodes in a wearable cardiac device in accordance with some embodiments of the present disclosure;

FIG. 24 is a process for determining orientation of therapy electrodes coupled to inertial motion units in a wearable cardiac device in accordance with some embodiments of the present disclosure;

FIG. 25 is a schematic and a process for determining orientation of therapy electrodes coupled to inertial motion units in a wearable cardiac device in accordance with some embodiments of the present disclosure;

FIG. 26 is a schematic and a process for determining orientation of therapy electrodes coupled to inertial motion units in a wearable cardiac device in accordance with some embodiments of the present disclosure;

FIG. 27 is a schematic and a process for determining orientation of therapy electrodes coupled to inertial motion units in a wearable cardiac device in accordance with some embodiments of the present disclosure; and

FIG. 28 is a process for determining a fall-off condition for therapy electrodes coupled to inertial motion units in a wearable cardiac device in accordance with some embodiments of the present disclosure.

### DETAILED DESCRIPTION

Wearable cardiac medical devices can include therapy electrodes that are configured to deliver therapeutic shocks to a patient. The proper placement and orientation of the therapy electrodes is advantageous to delivering effective cardiac therapy. Implementations herein provide advantages by allowing patients that are often required to place the therapy electrodes in the garments of the cardiac medical device themselves feedback, guidance, or confirmation that they have properly placed or oriented the electrodes. Such implementations are advantageous by having the electrodes be properly aligned or oriented or positioned as designed, expected or required for the proper application of the therapeutic devices.

In example implementations, garments associated with the wearable cardiac medical devices can include multiple pockets configured to receive therapy electrodes. Patients can be required to insert therapy electrodes into the pockets themselves on a daily basis, such as when reattaching the wearable cardiac medical device after a shower. Implementations herein allow for the patients to insert the therapy electrodes into the pockets with limited or no medical supervision. In this manner, the example features described herein allow for the electrodes to be properly inserted. For example, using the systems, techniques and devices herein, the inserted electrodes can therefore be properly aligned or oriented within the pocket.

Accordingly, the disclosed systems and methods provide for the verification of the facing orientation of electrodes in a garment worn by an ambulatory patient and can include the verification of facing orientation and/or anatomical placement of electrodes on an ambulatory patient. In some embodiments, systems and methods for the verification of facing orientation and/or anatomical placement of electrodes can utilize data received from one or more motion sensors integrated or attached to the therapy electrodes. The data is processed to determine motions signals that are used to determine the orientation for the plurality of therapy electrodes. Examples of motion sensors can include inertial motion units, accelerometers, and/or gyroscopes. In some embodiments, the motion data is processed and orientation information received from the sensors can be compared to determine if a sensor and corresponding therapy electrode is incorrectly oriented. One or more alerts can be generated and provided to the ambulatory patient (e.g., via a local user interface) or to a remote technician or other authorized caregiver (e.g., via a remote technician interface or a remote caregiver interface at a location that is remote from the patient's location) if the therapy electrode is incorrectly oriented by the ambulatory patient. Additionally, alerts confirming proper placement of the therapy electrodes can also be provided to the ambulatory patient wearing the wearable cardiac device or the remote technician. For example, the remote technician or caregiver, upon receiving the alert, can undertake one or more actions responsive to the alert. For example, the technician or caregiver can follow up with the patient after a predetermined duration (e.g., 4 -24 hours, 1-2 days, or 2-14 days) or send a message to the patient, or monitor the patient's actions without further action. For example, the follow up action can include asking the patient to visit their caregiver for adjustment of the wearable cardiac device, further training on proper use and assembly of the device, or participate in a live conversation or discussion about how to correct for the improper placement or orientation of the electrodes in the device.

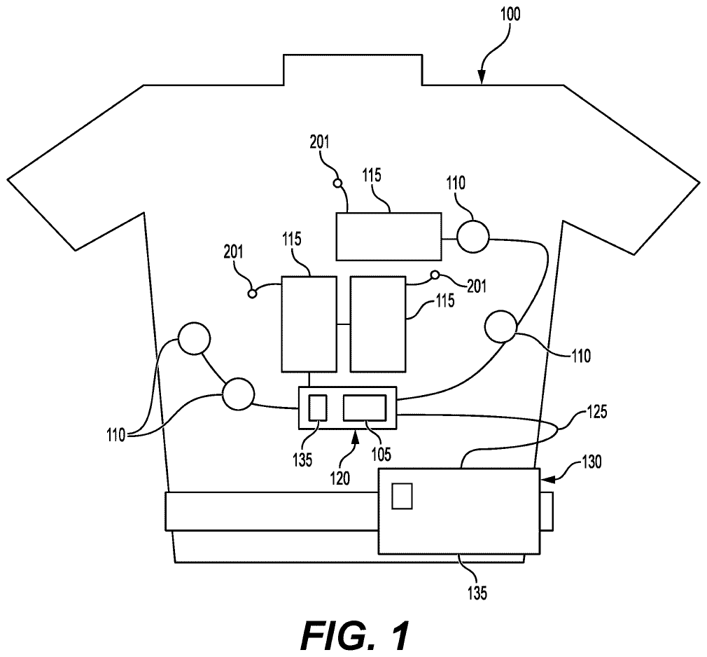

FIG. 1 illustrates a wearable cardiac device or wearable treatment device 100 configured for a subject to wear as a garment. The subject includes a person receiving health care, such as a subject who may or may not be under supervision of a doctor or health care provider. The subject may be in or out of a hospital setting, and the subject can engage in day to day life activities, at home, work, leisure, and play while wearing the treatment device 100.

Accordingly, the subject may be referred to as an ambulatory patient. Treatment device 100 includes monitoring, treatment and data transmission and processing capability, and can be worn as a vest, belt, shirt, or series of straps, garment, or undergarment for example. Treatment device 100 may include at least one power supply such as a battery, or other power supplies, including AC power supplies and uninterruptable power supplies. Treatment device 100 can monitor and treat cardiac ailments such as heart failure, as well as other medical conditions such as arrhythmias, pulmonary ailments, other heart irregularities, sleep disorders, and circulatory system deficiencies such as blockages.

In one embodiment, treatment device 100 includes dedicated control logic devices that collectively constitute a control system, such as at least one controller 105. Controller 105 can include programmable logic devices and arrays, application specific integrated circuits, hardware and software combinations, general purpose processors and dedicated controllers, for example. Further, treatment device 100 may include graphical user interfaces or other interfaces to provide output information and receive input information from a user. Controller 105 can be contained entirely within treatment device 100, or at least partially located external to treatment device 100. The controller 105 is configured to monitor cardiac physiological information (e.g., ECG data, cardiovibration data, among others) for cardiac abnormalities and initiating treatment of detected cardiac abnormalities. For instance, within the context of a wearable defibrillator, such critical functions include charging the capacitors to a particular voltage, digital sampling and analysis of ECG information and generation of the delivered energy waveform. For example, controller 105 includes one or more processors confirmed to execute a preconfigured firmware or software modules in accordance with the detection or the cardiac abnormalities and one or more treatment protocols responsive to such detected cardiac abnormalities.

Treatment device 100 can also include a plurality of sensors 110, 135. The plurality of sensors 110, 135 may include subject medical condition sensors 110, such as cardiac sensing electrodes, and subject activity sensors 135, such as motion sensors, inertial motion units, gyroscopes, or accelerometers. While four external medical condition sensors 110 are illustrated in FIG. 1, treatment device 100 can include more or less than four external medical condition sensors 110, and in some embodiments, sensors 110 include at least one internal sensor or external dry electrode. Sensors 110 may include at least one cardiac sensing electrode to detect a subject's cardiac information related to the subject's heartbeat or electrical activity of the subject's heart. Sensors 110 are configured for placement proximate to the subject, for example, about the subjects torso, chest, back, limbs, or neck, where they can sense information about the subject's bodily functions. In one embodiment, sensors 110 include a fingertip pulse oximeter that can generate a photoplethysmograph to measure blood flow, blood oxygen saturation, respiration, or hypovolemia. In other embodiments, sensors 110 can include sensors that monitor or measure wellness information indicative of a general wellness of the subject, such as pulse, breathing, temperature, blood pressure, or fatigue information, for example.

In one embodiment, the plurality of sensors 110, 135 includes subject activity sensors 135. In one embodiment, subject activity sensors 135 can include at least one accelerometer to detect subject movement, lack thereof, or positional orientation. Sensors 110, 135 that include subject activity sensors generally detect tangible medical or physical condition or information indicative of a subject's overall health, as well as statistically significant changes in measurements or conditions with time that may indicate changes in the subject's health, such as a worsening heart failure condition.

In some embodiments, the plurality of sensors 110 can include a plurality of motion sensors 201 that are coupled to the therapy electrodes or treatment electrodes 115. The motion sensors 201 can include anatomical placement sensors which may include anatomical placement accelerometers, inertial motion units, gyroscopes and the like. The anatomical placement sensors can include a circuit having at least one of a gyroscope, accelerometer and/or inertial motion unit. The motion sensors 201 can include one or more gyroscopes configured to generate at least one anatomical placement motion signal indicating at least one of a rotation direction, rotation angle and/or vibration. The motion sensors 201 can include at least one accelerometer which is configured to generate at least one anatomical placement motion signal indicating linear acceleration in along an x-axis, y-axis, or z-axis. In some embodiments, the motion sensors 201 can include an inertial motion unit that is configured to generate at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis.

Treatment device 100 may also include at least one treatment electrode 115. In one embodiment, treatment electrode 115 is configured to deliver shocks or electric current to the subject, such as a defibrillation shock applied to resuscitate a subject during cardiac arrest or another cardiac event. Treatment electrodes 115 may be housed in therapy pads that also include receptacles to house conductive fluid such as conductive gel. For example, treatment electrodes 115 may include dry treatment electrodes. In this example, prior to treatment, controller 105 can direct the receptacle to burst, releasing conductive fluid that contacts a surface of treatment electrode 115 as well as the subject's skin, enhancing the electrical connection between the subject and treatment electrode 115. The receptacles can be replaced after use.

In one embodiment, treatment electrodes 115 are formed from plates of metal or other conductive material having a conductive surface and configured for contact with the subject. The therapy electrodes may have generally circular, oval, rectangular, square, or other geometric forms with a generally continuous surface. In some embodiments, treatment electrodes 115 are formed from conductive wire or thread sewn into treatment device 100 in stitched, woven, or intertwined patterns, including a mesh pattern. In one embodiment, treatment device 100 includes at least one node 120 to connect or interface with sensors 110 and treatment electrodes 115. Node 120 may be located on a belt of treatment device 100 and can be part of or associated with controller 105 to facilitate communication between controller 105, sensors 110, 135 and treatment electrodes 115. In one embodiment, node 120 is a device to physically couple cables or wire 125 that connect controller 105, sensors 110, 135 treatment electrodes 115, and other treatment device 100 components, such as at least one monitor 130. FIG. 1 depicts three treatment electrodes 115, with one treatment electrode positioned proximate to the subject's chest, and two treatment electrodes 115 positioned proximate to the subject's back. This configuration can be used to provide shocks to the subject's heart during defibrillation treatment. Other configurations and positions of treatment electrodes 115 are possible for defibrillation and other treatments.

In one embodiment, treatment device 100 includes at least one subject activity sensor 135. For example, subject activity sensor 135 may include at least one accelerometer that can indicate accelerating and decelerating movements. For example, a subject wearing treatment device 100 can participate in normal activities, such as standing, walking, sitting, running, and generally moving about as part of day-to-day life when partaking in physical, labor, and leisure activities. Because of the nature of human movements, generally comprising short distance and short duration, accelerometers provide useful information about subject movement and activity. Controller 105 can use this information to determine if treatment is necessary or should be adjusted, if quality of life recommendations should be made to the subject (e.g., a suggestion to change dietary or activity habits,) or if a doctor should be consulted. In some embodiments, activity sensors 135 include single axis accelerometers as well as multi-axis sensors.

In one embodiment, the plurality of sensors 110, 135 include at least one cardiac sensing electrode 110, a subject activity sensor 135, such as an accelerometer, or other sensor configured to provide information to controller 105 relating to the subjects cardiac information (e.g., ECG), or activity wellness (e.g., motion or position). For example, sensor 135 can sense and provide information about the subject's body state—e.g., vertical, horizontal, lying down on left side, lying down on right side, moving in a recitative pattern, vibrating due to environmental causes such as during a car ride, convulsing due to health causes such as a cardiac event or seizure, accelerating, decelerating, falling, and treatment device 100 component acceleration or mechanical shock, (e.g., sensor 135 disconnects from the subject and falls or impacts the ground or a hard surface due to gravitational or other forces).

In one embodiment, treatment device 100 includes two activity sensors 135, such as accelerometers. For example, a first accelerometer can be located on node 120 and a second accelerometer can be located on monitor 130. In one embodiment, the first accelerometer is positioned on the subject's upper body, and the second accelerometer is positioned proximate to the subject's waist. Accelerometers or other activity sensors 135 may also be positioned on the subject's limbs. Activity sensors 135, including accelerometers, may include at least one position, force, or motion detector. In one embodiment, controller 105 uses information detected by multiple activity sensors 135, such as accelerometers to determine and predict subject activity, and to calibrate or verify the accuracy of sensors 110 and/or sensors 135. For example, one or more of sensors 110 may be tasked with determining the subject's heart beat, and may shift due to movement or be improperly positioned so that an inaccurate reduced heartbeat is sensed. In this example, activity sensors 135 may indicate that the subject is exercising and where an elevated heartbeat would be expected, while sensor 110 detects a reduced heart beat or no heart beat because it is improperly positioned on the subject. Controller 105 can identify this discrepancy and notify the subject, for example by a display on monitor 130, that one of sensors 110 should be repositioned. By processing sensed information and information received from the user, controller 105 may also determine that treatment device 100 components have been tampered with or damaged, and monitor 130 can display a notification of any such tampering or damage. In one embodiment, controller 105 is located together with monitor 130.

In some embodiments motion sensors 201 including an anatomical placement sensor circuit can be coupled to the therapy or treatment electrodes 115. For example, the anatomical placement sensor circuit can be disposed within controller 105, e.g., the circuit is implemented as part of the controller 105 through one or more firmware or software modules implemented by one or more processors of the controller 105. In some implementations, the anatomical placement sensor circuit can be disposed external to the controller 105, and under control of one or more processors of the controller 105. Further details of the controller 105 and manner of control of associated circuitry is described below.

In one embodiment, controller 105 evaluates activity sensor 135 information to determine the position of the subject and any corresponding applied forces. For example, activity sensor 135 can measure x, y, and z axis orientations of the subject. Controller 105 can use this information in a confidence based arrhythmia detection algorithm to accelerate or delay the timing of treatment based on past and present body motion or position history. Multiple activity sensors 135 permit separate evaluation of different subject movements and controller 105 evaluates subject movements to determine subject activity, create a real time and comprehensive subject medical record, and to recommend, apply, or adjust treatment regimens. The treatment applied can depend upon the diagnostic requirement of the subject's doctor and the condition of the subject (e.g., heart failure or congestive heart failure) that the doctor or the subject wishes to monitor.

In one embodiment, activity sensors 135 include at least one accelerometer to sense high sensitivity subject activity and wellness information, such as breathing or other generally subtle forms of motion such as body position (e.g., standing or prone). Sensors 110, 135 can detect and monitor physical activity and activity trends, body positions, and sleep conditions, such as sleep apnea. For example, sleep apnea may be deduced based on pulse oximetry and respiration measurements. Sensors 135 can also include at least one accelerometer to measure low sensitivity data such as mechanical shock.

In some embodiments, activity sensors 135 include at least one multi-axis accelerometer, or two three-axis accelerometers with one of the accelerometers mounted on a vest portion of treatment device 100 and another of the accelerometers mounted elsewhere on treatment device 100, such as a strap about the waist, or on monitor 130, which can include a visual display where the orientation of the visual display is controlled by the output of accelerometer.

In some embodiments, the controller 105 can, via one or more processors of the controller 105, also evaluate anatomical placement data generated by the anatomical placement sensors and motions sensors 201. This can include data indicating rotation direction, rotation angle, vibration, linear acceleration in the x-axis, y-axis, and/or z-axis, rotational acceleration in the x-axis, y-axis, and/or z-axis, and the like. Alternatively, the controller 105 can transmit the anatomical placement data to a remote server (e.g., located at a remote location from the location of the medical device) and/or one or more local additional processors (e.g., located within the medical device) for further processing. For example, the local processors can be within at least one monitor 130 as described in further detail below.

In one embodiment, treatment device 100 includes at least one monitor 130, which can include at least one touch screen, buttons, or other user interface such as a keyboard. The user interface may have multilingual audio and visual displays. Monitor 130 can also be remote from treatment device 100. Monitor 130 can display information to indicate that treatment device 100 is or is not properly configured about the subject. For example, monitor 130 can indicate that sensors 110, 135 are properly positioned and operational. Monitor 130 can attach to a belt or other portion of treatment device 100. In one embodiment, monitor 130 can be exposed, external to the subject's clothing, with at least some other treatment device components (e.g., sensors 110, 135) concealed beneath the subject's clothing. In one embodiment, treatment device 100 includes two monitors 130, with a first monitor housed on treatment device 100, and a second monitor remote to treatment device 100. The second monitor can communicate with controller 105. In one embodiment, the second monitor displays additional information that the first monitor does not display. For example, the second monitor can be part of a base station or a battery charger that includes a processor and memory. The second monitor can also be a personal computer monitor, (e.g., laptop, desktop, tablet, or mobile telephone monitor) configured to display the subject's historical medical record and other long term non-critical information, and the first monitor can be a dedicated application specific monitor that is housed on a belt of treatment device 100 configured to input and output core data related to the subject's present cardiac condition, general wellness, quality of life, and treatment regimen.

Monitor 130 may also include an alarm module. The alarm module can be audio, visual, tactile, or haptic, and can alert the subject as well as bystanders that treatment device 100 has applied, is applying, or will apply electric current or other treatment to the subject. The alarm module can also provide indicators of the subject's condition, such as heart or respiration rates, volume, or timing, or the subject's pulse, as well as heart failure indicators and coronary sounds.

In one embodiment, the alarm module provides an alarm after sensor 110 detects cardiac information about the subject, and before treatment device 100 applies treatment to the subject. The alarm module can also provide a further alarm after treatment has been applied to the subject. For example, the alarm module can alert first responders that at least one defibrillation shock has already been applied by treatment device 100. The alarm module can also alert bystanders or rescuers that it is safe to contact the subject after treatment has been applied, or that another round of treatment (e.g., another shock) is forthcoming. In one embodiment, the alarm module indicates that treatment will be applied. When the subject does nothing to abort the forthcoming treatment (such as depressing an abort switch or entering instructions via the user interface,) controller 105 can instruct treatment device 100 to administer an electric shock to the subject via one of treatment electrodes 115.

In some embodiments, the alarm module can be configured to provide a subject with an alert indicating that a least one electrode is improperly placed. Additionally, the alarm module can be configured to provide an improper electrode placement in garment alert based on a facing orientation of the therapy electrodes associated with the motion sensors 201 and/or a determination of the relative position of each therapy electrode relative to another therapy electrode.

As illustrated in FIG. 1, a first activity sensor 135 such as an accelerometer can be located in front of the subject, for example attached to monitor 130, and a second activity sensor 135, such as an accelerometer is located in back of the subject, for example attached to a belt of treatment device 100. Other configurations of accelerometers are possible, in front, in back, and on the sides of the subject, and attached to different belts, straps, or other components of treatment device 100. Wire 125 allows communication and data transfer between activity sensor 135, medical condition sensors 110, and controller 105 via node 120.

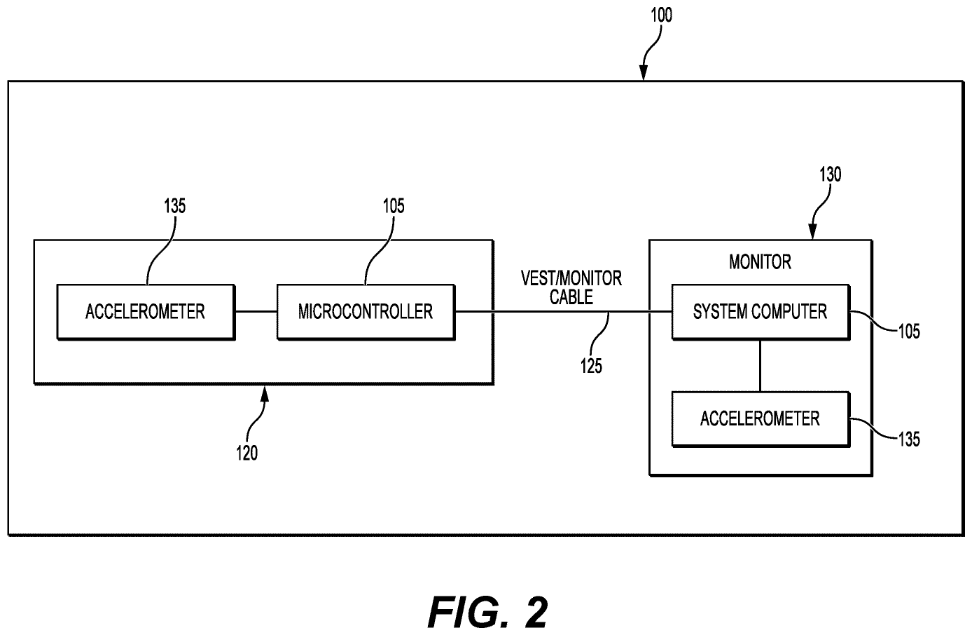

FIG. 2 depicts a block diagram of treatment device 100. As illustrated in FIG. 2, controller 105 includes a microcontroller and a system computer, with the microcontroller associated with node 120 and the system computer associated with monitor 130, and with wire 125 connecting the microcontroller with the system computer. Different configurations are possible. For example, more than one logic device can collectively constitute controller 105, and controller 105 may be part of treatment device 100. In one embodiment, at least some logic devices of controller 105, such as the system computer, are located external to treatment device 100. For example, both monitor 130 and the system computer can be separate from treatment device 100. Such external components may communicate with the microcontroller or other elements of controller 105 that are part of or housed on treatment device 100 via wire 125 or other connections, both wired and wireless. In one embodiment, the microcontroller process real time information related to the subject's cardiac information, quality of life, general wellness, and treatment regimen; and the system computer processes information related to the subject's long term medical history. For example, controller 105 can provide information to a remote computer via a wireless transmission to generate a comprehensive real time medical history of the subject when, for example, the subject wears treatment device 100 for any period of time. This medical history information may be stored in memory that is part of treatment device 100, or remotely, for example in a hard drive of a computer in a doctor's office. The system computer and the microcontroller can exchange information and instructions regarding treatment application and adjustment. In one embodiment, controller 105 communicates a message to a physician, responder, bystander or the subject to indicate that treatment is imminent, being provided, or has already been provided.

In one embodiment, controller 105 communicates with a central server that is external to treatment device 100. For example, sensed indicators of heart failure can be wired or wirelessly downloaded to a central server for processing, and presented to a doctor for review and analysis. This information can be tailored to a doctor's needs, for example to generate alerts and notifications.

In one embodiment, a first activity sensor 135, such as an accelerometer, is attached to node 120 and a second activity sensor 135, such as another accelerometer, is attached to monitor 130. Sensed information from both of these sensors 135 can be transferred to controller 105, which can be physically attached to treatment device 100, or remote from treatment device 100. In one embodiment, treatment device 100 includes two accelerometers to determine parameters such as subject body position, body movement, and body acceleration, and to perform self-diagnostics. Monitor 130 can contain either a high-G or a low-G accelerometer, or both. In one embodiment, a high-G low-sensitivity accelerometer can detect subject and equipment physical shock to determine if treatment device 100 is damaged. Activity sensors 135 can detect movement and orientation of the subject. In one embodiment, controller 105 processes information from two activity sensors 135, such as accelerometers to identify subject activity.

In one embodiments, motion sensors 201 can include motion information for one or more therapy electrodes in wired communication with a respective motion sensor. As described above, anatomical placement data can be generated by anatomical placement accelerometers, anatomical placement inertial motion units, and/or anatomical placement gyroscopes, which can be integrated with a corresponding therapy electrode.

Processing of accelerometer data and/or anatomical placement data can be performed by the microcontroller or the system computer. Accelerometers can indicate change in the subject's velocity. For example, the subject can have an activity level when conscious that includes changes in both velocity and direction. By contrast, an unconscious subject may have little or no change in body motion. Other activity sensors 135 (e.g., gyroscope, magnetometer, hall-effect devices, pedometers, global positioning systems, and other force motion or position sensors) can indicate motion or lack of motion. Outputs from sensors 135 may be integrated, compared or differentiated by controller 105 to predict subject activity, and reduce interference or error signals.

Output from motion sensors 201 can be processed or routed by controller 105 to determine anatomical placement motion signals.

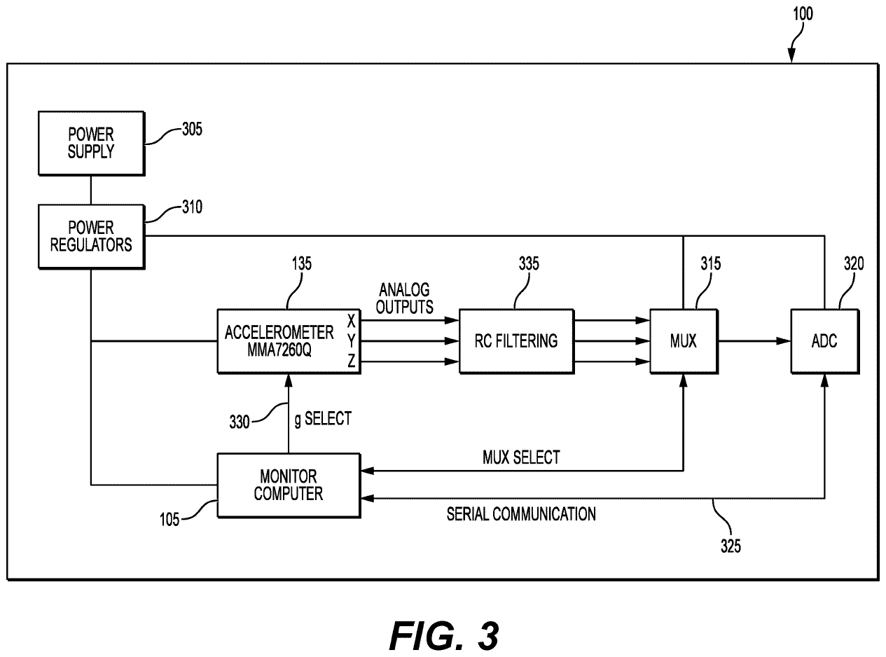

FIG. 3 depicts a block diagram of a treatment device 100. In one embodiment, AC or DC power supply 305 (e.g., a power cord to AC main lines, or a battery) can power treatment device 100 components, such as controller 105, monitor 130, and sensors 110, 135. At least one power regulator 310 can control the power from power supply 305.

In one embodiment, controller 105 controls various system parameters such as activity sensor sensitivity, motion sensor sensitivity, multiplexer (MUX) 315 channel select, the analog to digital converter (ADC) 320, and serial communication with controller 105 via serial communication bus 325 to acquire data from activity sensors 135 and motion sensors 201 and to display this information at monitor 130. MUX 315 and ADC 320 can be internal to controller 105, or can be separate components. In one embodiment, activity sensors 135 include a Freescale Semiconductor MMA7260Q three axis low-g micromachined accelerometer. The g-select control line 330 coupled to controller 105 and the accelerometer allows the sensitivity to be varied from, for example, 1.5 g to 6 g. A high-G low sensitivity accelerometer can also be used to allow subject/equipment shock to be detected. Resistor-capacitor (RC) filter 335 can connect to outputs of the accelerometer to minimize clock noise from the accelerometer internal switched capacitor filter circuit. Controller 105 can control select lines of multiplexor 315 and may allow each axis output of the accelerometer to be switched to the Analog to Digital Converter (ADC) 320 input. Controller 105 can also control ADC 320 via a serial interface. In one embodiment, sensors 110, 135, controller 105, and monitor 130 sense, process, and display other information such as sensed cardiac information, sensed general wellness information, and subject inputted self assessment entries including quality of life information.

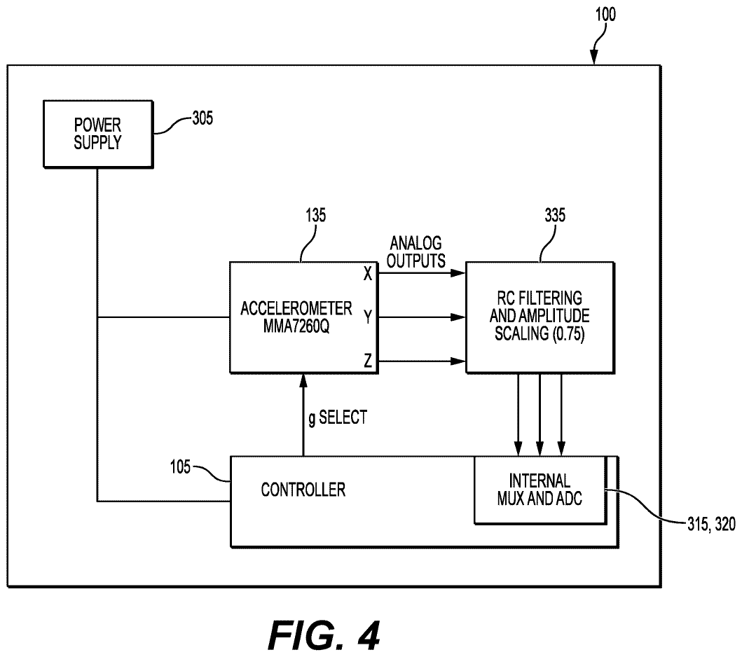

FIG. 4 depicts an alternative block diagram of a treatment device 100 where controller 105 acquires information from activity sensor 135, such as an accelerometer, and motion sensor 201 configured to include anatomical placement motion sensors. Power supply 305 can be used to power the components of treatment device 100. Activity sensor 135 can include a Freescale Semiconductor MMA7260Q three axis low-g micromachined accelerometer. Controller 105 controls the g-select lines that again can allow the sensitivity to be varied from, for example, 1.5 g to 6 g. RC filter 335 as well as amplitude scaling can be applied to each of the accelerometer outputs. In one embodiment, MUX 315 and ADC 320 are internal to controller 105 the analog outputs of the accelerometer are interfaced digitally directly to the controller 105.

In one embodiment, controller 105 detects an arrhythmia by assigning various confidence coefficients or weighting values to the various sensors 110, 135) that communicate with controller 105. In one embodiment, this is done prior to controller 105 determining a confidence level that detected motion indicates true motion, and not a false positive motion indication due, for example, to an incorrectly placed or dropped activity sensor. For example, controller 105 can separately analyze two independent ECG data streams from sensors 110 to extract heart rate, morphology, frequency information, general wellness, and other information. Controller 105 can perform additional analysis, independently on each channel, to analyze the signal for noise contamination that may result from subject motion or biological signals such as muscle noise. Secondary inputs to the basic detection algorithm can include a subject response button or override switch, where for example the subject indicates that they are in motion, and inputs from activity sensors 135. In one embodiment, controller 105 determines that the lack of response from the subject, for example, by not pressing a subject response button (e.g., an abort switch,) that can be part of treatment device 100, means that the subject is unconscious.

In one embodiment, a weighting value is assigned to each sensor 110, 135 and the response button to make the decision that a treatable arrhythmia condition exists. In addition, the weighting values can be used to manipulate or adjust the timing and nature of therapy delivered by therapy electrodes 115.

During use by a subject, there may be instances where a first ECG channel contains noise and a second ECG channel is clean. For example two pairs of sensors 110 can independently obtain ECG signals, with one pair being contaminated with artifacts and the other being clean. The two ECG signals can be obtained simultaneously or sequentially, and can be transmitted to controller 105 via the same or different communication channels (e.g., wire 125). In one embodiment, controller 105 places more weight on the clean ECG channel. For example, to enhance a confidence level of the sensed information, a weighting can be assigned that would delay delivery of treatment by treatment electrodes 115 while sensors 135 and controller 105 determine if there is subject motion.

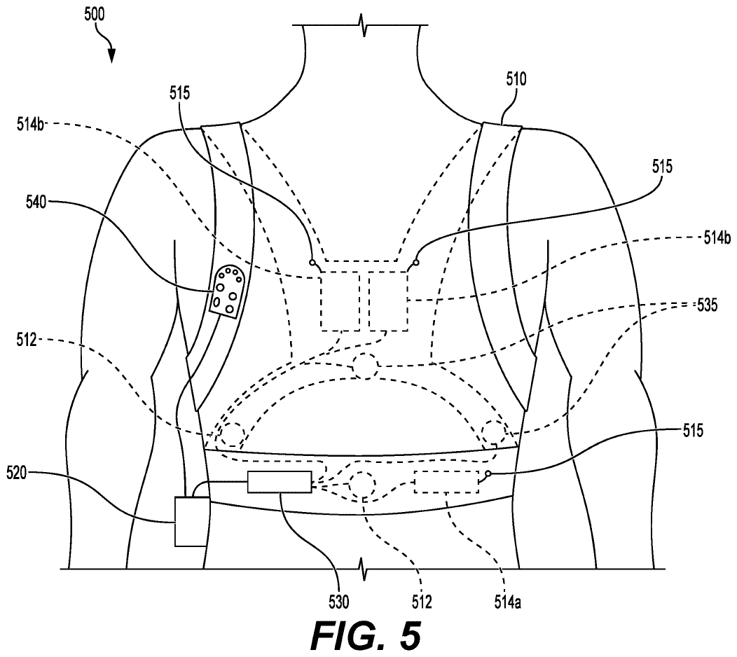

FIG. 5 illustrates a medical monitoring and treatment device, such as a Life Vest® Wearable Cardioverter Defibrillator available from ZOLL Medical Corporation of Chelmsford, Mass. Illustrated is a medical monitoring and treatment device 500 analogous to wearable cardiac device or wearable treatment device 100 of FIGS. 1-4. As shown, the medical monitoring and treatment device 500 includes a harness 510 having a pair of shoulder straps and a belt that is worn about the torso of a patient. The harness 510 is typically made from a material, such as cotton, nylon, spandex, or antron that is breathable, and unlikely to cause skin irritation, even when worn for prolonged periods of time.

The medical monitoring and treatment device 500 includes a plurality of electrocardiogramsing electrodes 512 that are disposed by the harness 510 at various positions about the patient's body and electrically coupled (wirelessly or by a wired connection) to a portable treatment controller 520 via a connection pod 530. The plurality of ECG sensing electrodes 512 are used by the portable treatment controller 520 to monitor the cardiac function of the patient and generally include a front/back pair of ECG sensing electrodes and a side/side pair of ECG sensing electrodes. It should be appreciated that additional ECG sensing electrodes may be provided, and the plurality of ECG sensing electrodes 512 may be disposed at varying locations about the patient's body. In addition, the plurality of ECG electrodes 512 may incorporate any electrode system, including conventional stick-on adhesive electrodes, dry-sensing capacitive ECG electrodes, radio transparent electrodes, segmented electrodes, or one or more long term wear electrodes that are configured to be continuously worn by a patient for extended periods (e.g., 3 or more days.

The medical monitoring and treatment devices disclosed herein may incorporate sundry materials arranged in a variety of configurations to maintain a proper fit with the patient's body Thus embodiments are not limited to the configuration and materials described above with reference to FIG. 5.

The medical monitoring and treatment device 500 also includes a plurality of therapy electrodes 514 that are electrically coupled to the portable treatment controller 520 via the connection pod 530 and which are capable of delivering one or more therapeutic defibrillating shocks to the body of the patient, if it is determined that such treatment is warranted. As shown, the plurality of therapy electrodes 514 includes a first therapy electrode 514 a that is disposed on the front of the patient's torso and a second therapy electrode 514b that is disposed on the back of the patient's torso. The second therapy electrode 514b includes a pair of therapy electrodes that are electrically coupled together and act as the second therapy electrode 514b. The use of two therapy electrodes 514a, 514b permits a biphasic shock to be delivered to the body of the patient, such that a first of the two therapy electrodes can deliver a first phase of the biphasic shock with the other therapy electrode acting as a return, and the other therapy electrode can deliver the second phase of the biphasic shock with the first therapy electrode acting as the return.

Also illustrated are motions sensors 515 which are integrated with a corresponding therapy electrode. The motion sensors 515 can include one or more of anatomical placement accelerometers, anatomical placement gyroscopes, anatomical placement inertial motion units, and the like. The motion sensors 515 can be configured to determine motion-based data for their respective therapy electrodes.

The connection pod 530 electrically couples the plurality of ECG sensing electrodes 512 and the plurality of therapy electrodes 514 to the portable treatment controller 520, and may include electronic circuitry. For example, in one implementation the connection pod 530 includes signal acquisition circuitry, such as a plurality of differential amplifiers to receive ECG signals from different ones of the plurality of ECG sensing electrodes 512 and to provide a differential ECG signal to the portable treatment controller 520 based on the difference therebetween. The connection pod 530 may also include other electronic circuitry, such as a motion sensor or accelerometer by which patient activity may be monitored.

In some embodiments, both the first therapy electrode 514a and the second therapy electrode 514b are disposed on the front of the patient's torso. For example, the first therapy electrode 514a may be located at external to the apex of the heart and the second therapy electrode 514b may be located along the parasternal line. Thus embodiments are not limited to a particular arrangement of therapy electrodes 514. In some embodiments, both the first therapy electrode 514a, and second therapy electrode 514b can each be integrated with a respective motion sensor 515. In some embodiments, all or any subset of the therapy electrodes 514 can be integrated with a motion sensor 515.

In some embodiments, the plurality of ECG sensing electrodes 512 are positioned and paired such that artifacts generated from electrical activity are decreased. In other embodiments, the electronic circuitry included in the portable treatment controller 520 may equalize artifacts measured at electrodes by changing a gain or impedance.

As shown in FIG. 5, the medical monitoring and treatment device 500 may also include a user interface pod 540 that is electrically coupled to the portable treatment controller 520. The user interface pod 540 can be attached to the patient's clothing or to the harness 510, for example, via a clip (not shown) that is attached to a portion of the interface pod 540. Alternatively, the user interface pod 540 may simply be held in a person's hand. The user interface pod 540 typically includes one or more actionable user interface elements (e.g., one or more buttons, a fingerprint scanner, a touch screen, microphone, etc. . . . ) by which the patient, or a bystander can communicate with the portable treatment controller 520, and a speaker by which the portable treatment controller 520 may communicate with the patient or the bystander. In certain models of the Life Vest® Wearable Cardioverter Defibrillator, the functionality of the user interface pod 540 is incorporated into the portable treatment controller 520. In some embodiments, the interface pod 540 can be configured to provide an alert.

Where the portable treatment controller 520 determines that the patient is experiencing cardiac arrhythmia, the portable treatment controller 520 may issue an audible alarm via a loudspeaker (not shown) on the portable treatment controller 520 and/or the user interface pod 540 alerting the patient and any bystanders to the patient's medical condition. The portable treatment controller 520 may also instruct the patient to press and hold one or more buttons on the portable treatment controller 520 or on the user interface pod 540 to indicate that the patient is conscious, thereby instructing the portable treatment controller 520 to withhold the delivery of one or more therapeutic defibrillating shocks. If the patient does not respond, the device may presume that the patient is unconscious, and proceed with the treatment sequence, culminating in the delivery of one or more defibrillating shocks to the body of the patient.

The portable treatment controller 520 generally includes at least one processor, microprocessor, or controller, such as a processor commercially available from companies such as Texas Instruments, Intel, AMD, Sun, IBM, Motorola, Freescale and ARM Holdings. In one implementation, the at least one processor includes a power conserving processor arrangement that comprises a general purpose processor, such as an Intel® PXA270 processor and a special purpose processor, such as a Freescale™ DSP56311 Digital Signal Processor, which is incorporated by reference herein in its entirety. The at least one processor of the portable treatment controller 520 is configured to monitor the patient's medical condition, to perform medical data logging and storage, and to provide medical treatment to the patient in response to a detected medical condition, such as cardiac arrhythmia.

Although not shown, the medical monitoring and treatment device 500 may include additional sensors, other than the ECG sensing electrodes 512, capable of monitoring the physiological condition or activity of the patient. For example, sensors capable of measuring blood pressure, heart rate, heart sounds, thoracic impedance, pulse oxygen level, respiration rate, and the activity level of the patient may also be provided.

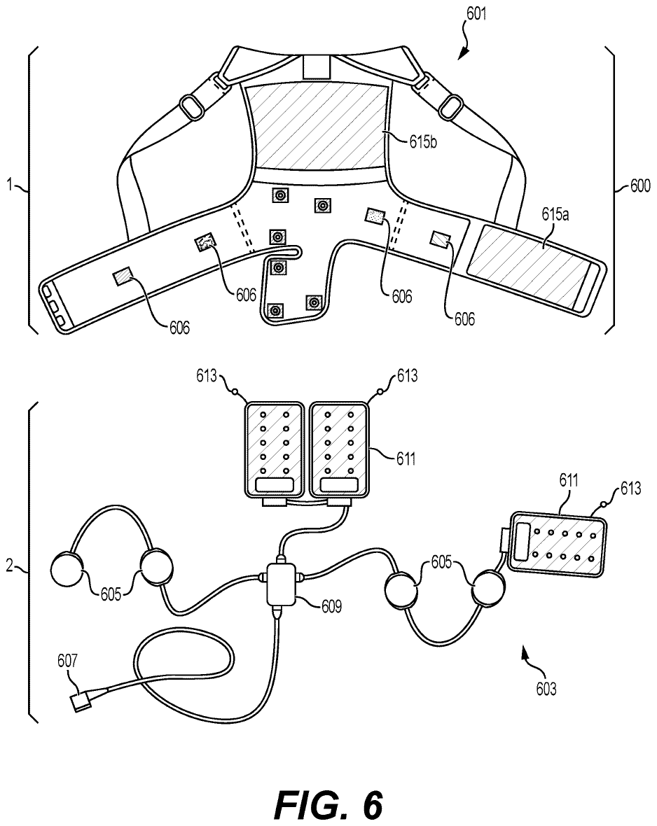

FIG. 6 provides a schematic example of a wearable cardiac device that includes electrode placement verification components configured for verifying the placement and orientation of therapy electrodes on an ambulatory patient. As shown in FIG. 6, the wearable cardiac device 600 can include a garment 601 and an electrode belt 603. The garment 601 can be configured to be worn on a torso of an ambulatory patient and include one or more therapy electrode pockets 615a 615b which are configured to receive and house therapy electrodes of the electrode belt 603. The garment 601 can be configured to be worn under the clothing of a subject and hold sensors and therapy components in alignment with predetermined positions on a subject's body. For example, the garment 601 can be structured such that the therapy electrode pockets 615a, 615b align the therapy electrodes to the back and lower left side of the subject's body.

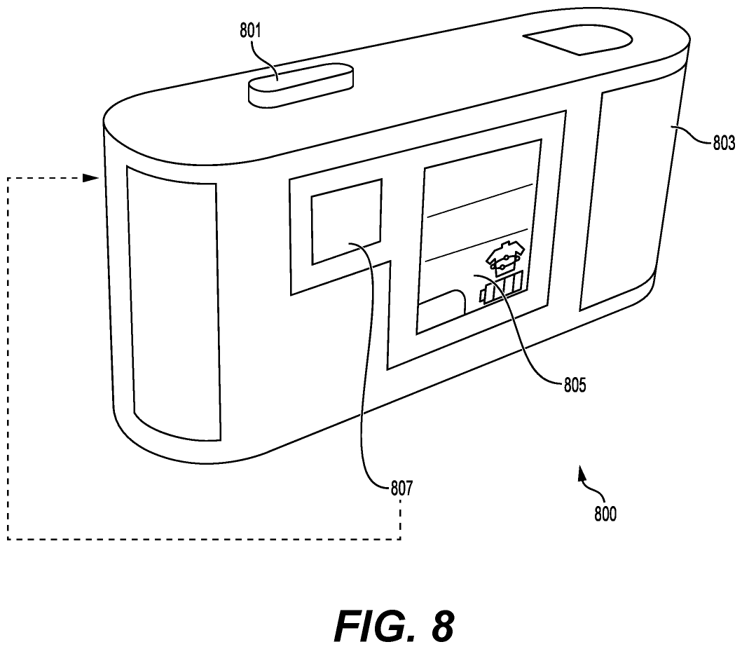

The electrode belt 603 can be configured to assemble into the garment 601. The electrode belt 603 can include one or more sensors, a vibration box, and/or therapy electrodes (also known as therapy pads). Sensors 605 can include echocardiogram sensors and the like. In some embodiments, the sensors 605 can be configured to sense ECG signals of a patient. The sensors 605 can be configured to be disposed at one or more predetermined ECG sensing locations 606 within the garment. Sensors 605 can include additional sensors for determining heart rate, and the like. The electrode belt 603 can also include a vibration box or alert module 609 configured to notify a subject that the device 600 is preparing to apply a treatment to the subject. One or more components of the electrode belt 603 can be connected to a monitor (e.g., see FIG. 8) via a connector 607.

Therapy pads or therapy electrodes 611 can be configured to deliver a therapeutic shock to a subject. The therapy electrodes 611 can be configured to be disposed within a therapy electrode pocket 615a, 615b of the garment 601 when the electrode belt 603 is assembled into the garment 601.

One or more of the therapy pads or therapy electrodes 611 in the electrode belt 603 can include one or more motion sensors 613 including anatomical placement accelerometers, anatomical placement gyroscopes and/or anatomical placement inertial motion units. In some embodiments, the motion sensors 613 are associated with and integrated with a corresponding therapy electrode 611. For example, the motion sensors 613 can be placed on the therapy electrode 611, or the motion sensors 613 can be coupled to the therapy electrode 611 via a connecting wire and the like. The motion sensors 613 can be configured to produce anatomical placement motion signals. Based on the signals generated by the motion sensors 613 one or more processors can determine a facing orientation for the associated therapy electrode. Additionally, or alternatively, one or more processors can determine a relative position of each therapy electrode relative to another therapy electrode based on the signals generated by the motion sensors 613 associated with each therapy electrode. Various methods for determining a facing orientation and/or relative positioning are discussed herein.

A facing orientation, as used herein, can refer to the orientation and/or positioning of each therapy electrode within a corresponding therapy electrode pocket and/or against the patient's skin. The therapy electrodes can be configured to have directionality, in that a particular face or side of the therapy electrode can be configured to face a subject to apply the therapeutic treatment to the patient. Accordingly, when the therapy electrodes of the belt are engaged with the garment and inserted into their respective therapy electrode pockets, the therapy electrodes are required to be facing the proper orientation such that therapeutic treatments can be applied properly.

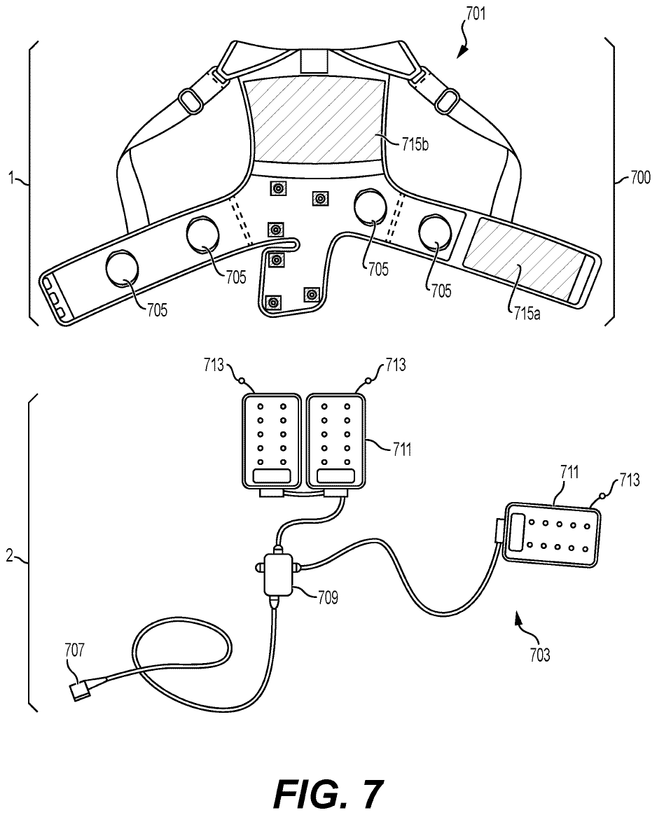

FIG. 7 provides a second schematic example of a wearable cardiac device that includes electrode placement verification components configured for verifying the placement and orientation of therapy electrodes on an ambulatory patient. As shown in FIG. 7, the wearable cardiac device 700 can include a garment 701 and an electrode belt 703.

The garment 701 can be configured to be worn on a torso of an ambulatory patient and include one or more therapy electrode pockets 715a 715b which are configured to receive and house therapy electrodes of the electrode belt 703. The garment 701 can be configured to be worn under the clothing of a subject and hold sensors and therapy components in alignment with predetermined positions on a subject's body. For example, the garment 701 can be structured such that the therapy electrode pockets 715a, 715b align the therapy electrodes to the back and lower left side of the subject's body. The garment 701 can also include one or more sensors 705 that are integrated into and/or permanently coupled into the garment. In some implementations, the sensors 705 cannot be removed by a subject. The sensors 705 can include ECG sensors configured to sense ECG signals of the subject.

The electrode belt 703 can be configured to assemble into the garment 701. The electrode belt 703 can include one or more sensors, a vibration box, and/or therapy electrodes (also known as therapy pads). The electrode belt 703 can also include a vibration box or alert module 709 configured to notify a subject that the device 700 is preparing to apply a treatment to the subject. One or more components of the electrode belt 703 can be connected to a monitor (e.g., see FIG. 8) via a connector 707.

Therapy pads or therapy electrodes 711 can be configured to deliver a therapeutic shock to a subject. The therapy electrodes 711 can be configured to be disposed within a therapy electrode pocket 715a, 715b of the garment 701 when the electrode belt 703 is assembled into the garment 701.

One or more of the therapy pads or therapy electrodes 711 in the electrode belt 703 can include one or more motion sensors 713 including anatomical placement accelerometers, anatomical placement gyroscopes and/or anatomical placement inertial motion units. In some embodiments, the motion sensors 713 are associated with and integrated with a corresponding therapy electrode 711. For example, the motion sensors 713 can be placed on the therapy electrode 711, or the motion sensors 713 can be coupled to the therapy electrode 711 via a connecting wire and the like. The motion sensors 713 can be configured to produce anatomical placement motion signals. Based on the signals generated by the motion sensors 713 one or more processors can determine a facing orientation for the associated therapy electrode. Additionally, or alternatively, one or more processors can determine a relative position of each therapy electrode relative to another therapy electrode based on the signals generated by the motion sensors 713 associated with each therapy electrode. Various methods for determining a facing orientation and/or relative positioning are discussed herein.

FIG. 8 illustrates a schematic diagram for a controller 800 analogous to controller 105, which can also be referred to as a monitor. The controller 800 can be communicatively coupled to a wearable cardiac device such as device 600 or device 700 via a connector 607, 707 and the like. The controller 800 can include a connector 801 configured to receive connector 607, 707 and the like. The controller 800 can include a battery 803 that is configured to power the controller. In some embodiments, the battery 803 can be rechargeable. The controller 800 can include a touchscreen 805 that is configured to communicate with the subject and/or a medical professional. In some embodiments, the touchscreen 805 can be interactive and receive input from the subject. The touchscreen 805 can be configured to display messages regarding device operation and/or receive subject input. In some embodiments, the touchscreen 805 display can be turned on and/or off by input to response buttons 807. The response buttons 807 can be configured to include one or more LEDs. For example, the LEDs can be configured to light a solid red color when the device senses that the subject's heart is in a life-threatening rhythm. In some embodiments, if the subject does not interact with the controller 800 via the response buttons 807, the device may provide a therapeutic defibrillation shock to the subject.

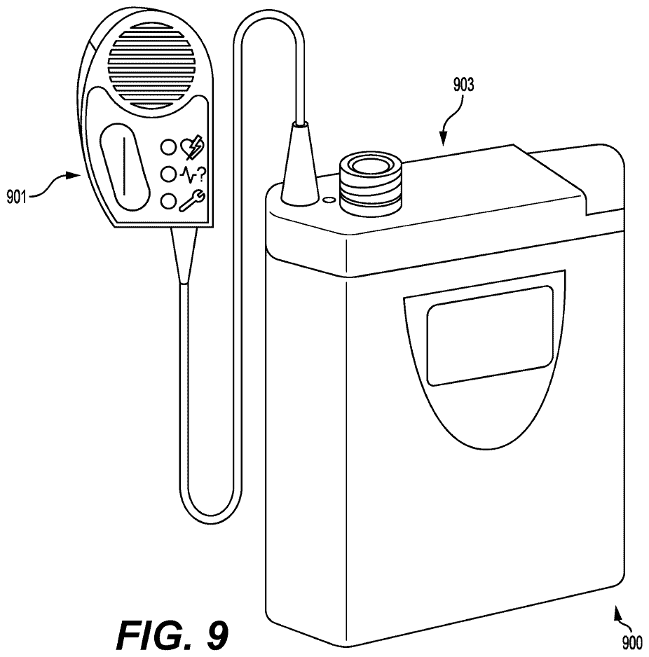

FIG. 9 illustrates a second schematic diagram of a controller 900 analogous to controller 800 and controller 105. Similar to the controller illustrated in FIG. 8, the controller 900 can include an alarm module 901 and a wearable cardiac device monitor 903. The controller 900 can be coupled to the wearable cardiac device such as device 600 or device 700 via a connector.

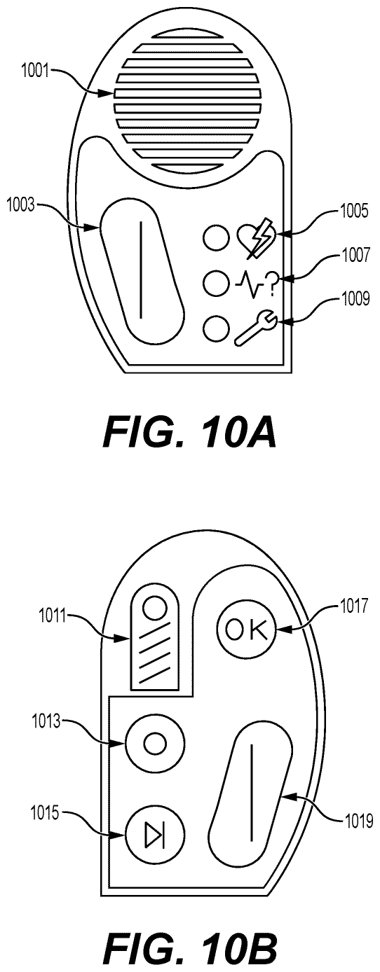

FIGS. 10A and 10B illustrate schematic diagrams for an alarm module 1000 analogous to alarm module 901. FIGS. 10A and 10B illustrate front and rear views of an alarm module 1000, respectively. As illustrated in FIGS. 10A and 10B, the alarm module can be connected to a controller such as controller 900 in FIG. 9. In some embodiments, the alarm module 1000 can be clipped onto the belt or breast pocket. The alarm module can provide the subject with alerts regarding their cardiac activity and/or the operational status of the wearable cardiac device. In some embodiments, alerts provided by the alarm module can include lights, voice messages, display panels and the like. In some embodiments, lights on the alarm module 1000 can be accompanied by one or more messages displayed in a controller such as controller 800, 900.

As shown in FIG. 10A, the alarm module 1000 can include a speaker 1001, response buttons 1003, and various icons and associated LED lights 1005, 1007, 1009. For example, a red light 1005 can be accompanied with a symbol of a heart and be configured to indicate an abnormal rhythm. A yellow light 1007 can be configured to indicate that one or more ECG signals are unclear.

In some embodiments, a flashing yellow light 1009 accompanied by a wrench can be configured to indicate that the device requires attention or service. In some embodiments, a determination that the at least one of a facing orientation of the plurality of therapy electrodes or a relative position of each therapy electrode relative to another therapy electrode of the plurality of therapy electrodes does not meet a predetermined electrode placement criterion relating to the proper placement of the electrodes within the garment, can result in a generation of an improper electrode placement in garment alert. The improper electrode placement in garment alert can be provided to a subject by the flashing yellow light 1009.

As shown in FIG. 10B, the alarm module can also include an okay button 1017, one or more response buttons 1019, a clip 1011, a record button 1013 and/or a transmit button 1015.

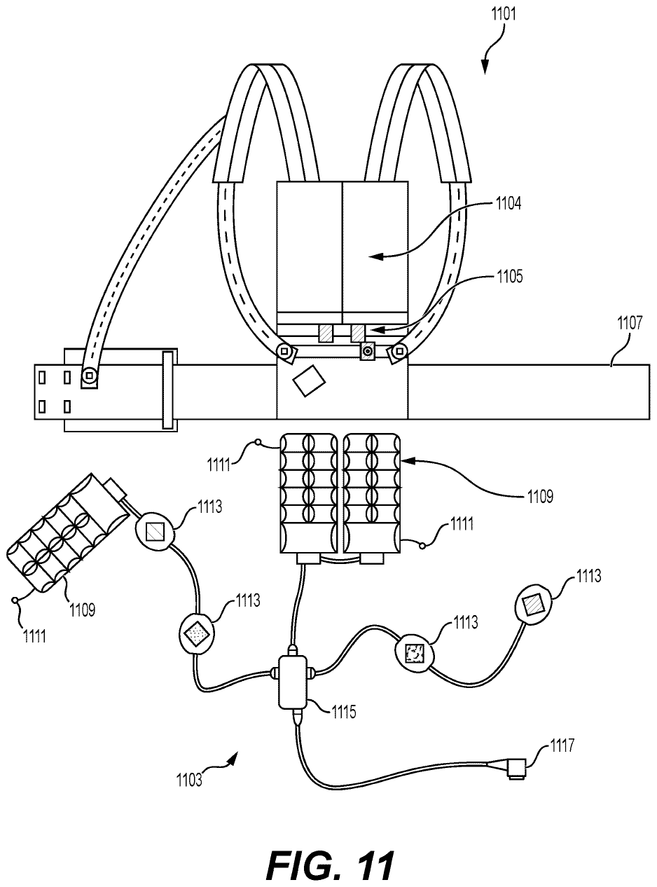

FIG. 11 provides an illustration for a first garment style for a wearable cardiac device. The first garment 1101 can be associated with an electrode belt 1103. The wearable cardiac device can be configured such that the garment 1101 is configured to receive the electrode belt 1103. As shown in FIG. 11, a garment 1101 can include a belt component 1107, one or more color coordinated portions configured to guide a user to assemble the electrode belt 1103 into the garment 1101. For example, the garment 1101 can include a first fabric color around the therapy electrode pockets 1104 that is configured to receive corresponding therapy electrodes 1109 and their associated motion sensors 1111 on the electrode belt 1103. For example, the therapy electrodes 1109 can also be of the same first fabric color. The electrode belt 1103 can include additional sensors 1113, a controller 1115 and a connector 117, analogous to that for the implementations discussed above. The garment 1101 can also include a plurality of garment attachment features that are configured to secure one or more components of the electrode belt 1103 to the garment 1101. For example, the garment attachment features can include hook and loop fasteners, snap buttons, or seams. In the illustrated embodiment, the garment attachment features include snap buttons or snap tabs that are configured to secure the therapy electrodes 1109 in the electrode belt 1103 within their respective therapy electrode pockets 1104 when the electrode belt 1103 is assembled into the garment 1101. In some embodiments, the garment attachment features can be color-coded to provide visual guidance to a subject. In some embodiments, the fabric color for the garment attachment feature can be different from the fabric color of portions of the garment configured for receiving the electrode belt.

In some embodiments, garment attachment features for ECG sensing electrodes can be configured to use hook and loop fasteners and the like. In some embodiments, the garment attachment features for ECG sensing electrodes can include one or more seams configured to irreversibly couple the ECG sensing electrodes to the garment. In some embodiments, the garment attachment features can include snap buttons and the like configured to hold the ECG sensing electrodes or therapy electrodes and the like.

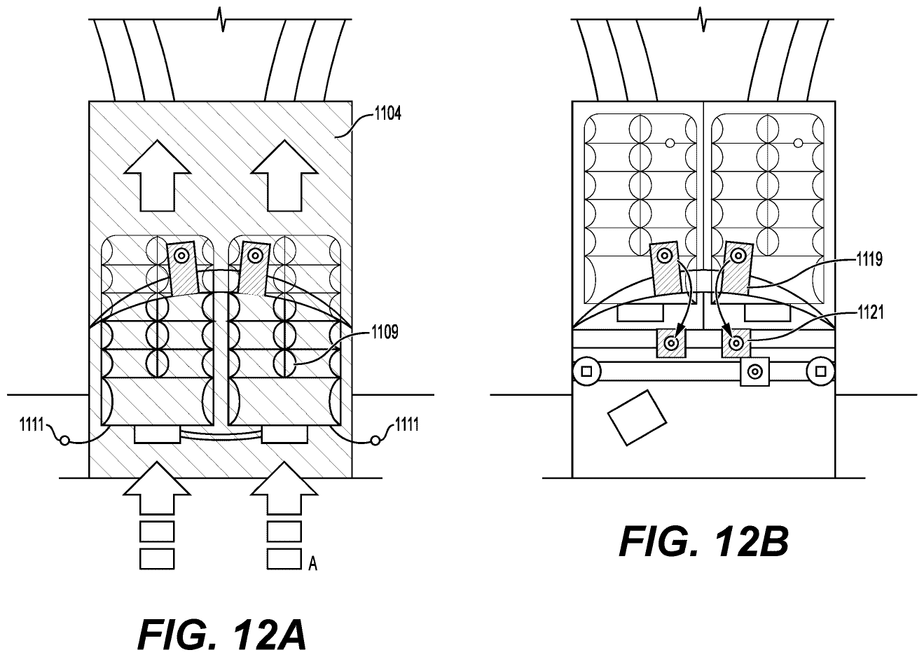

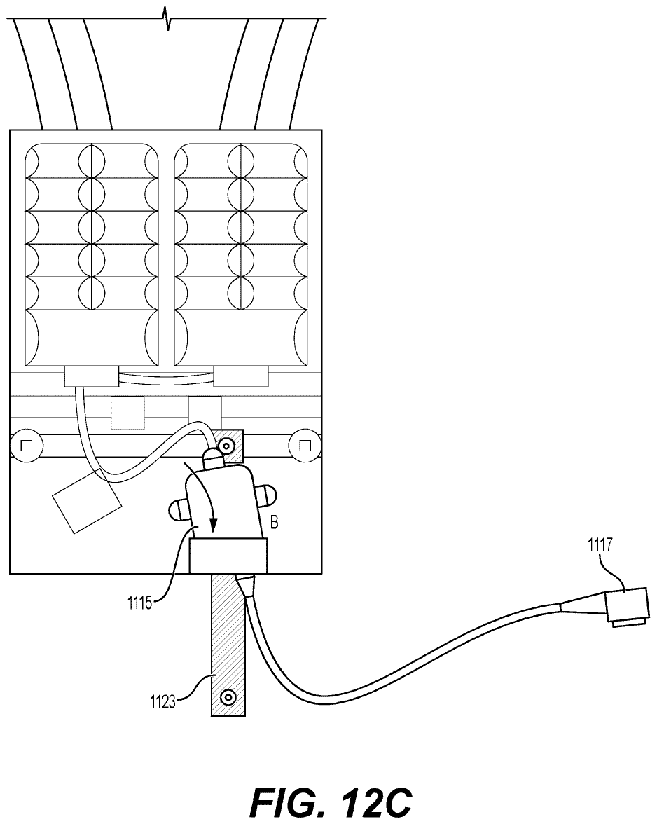

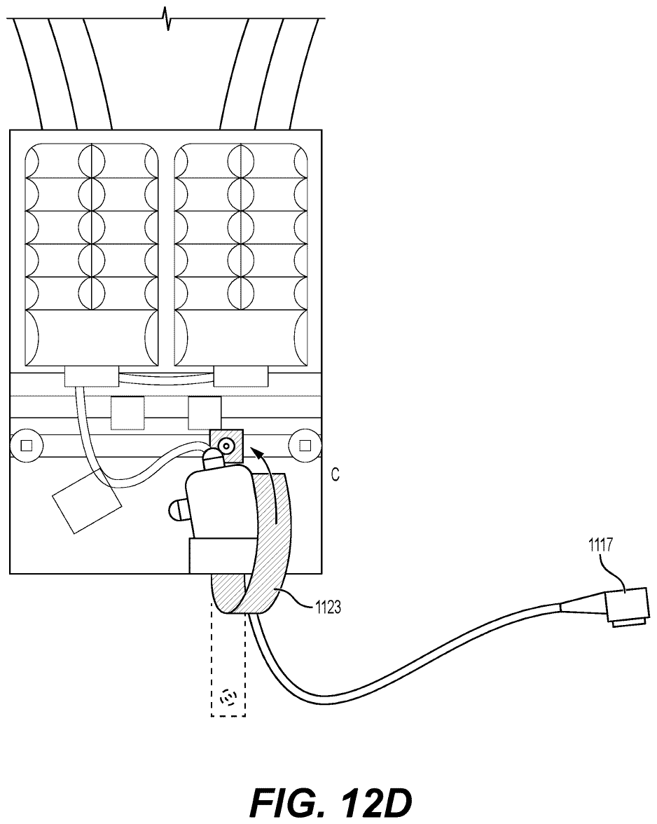

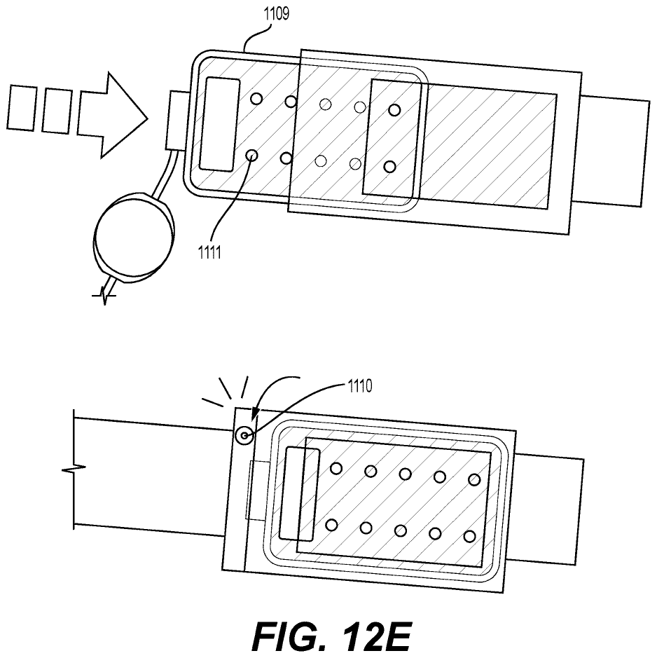

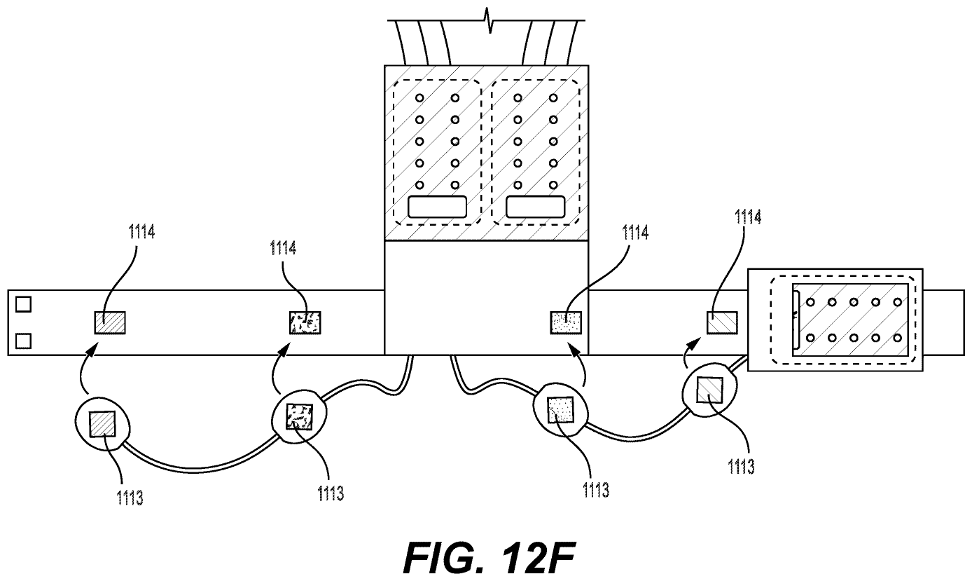

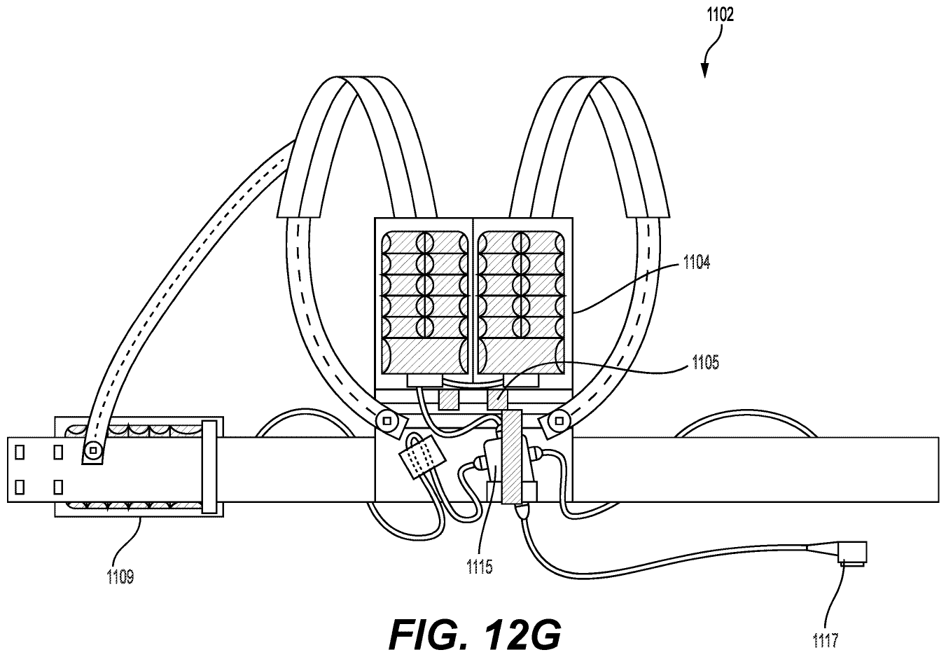

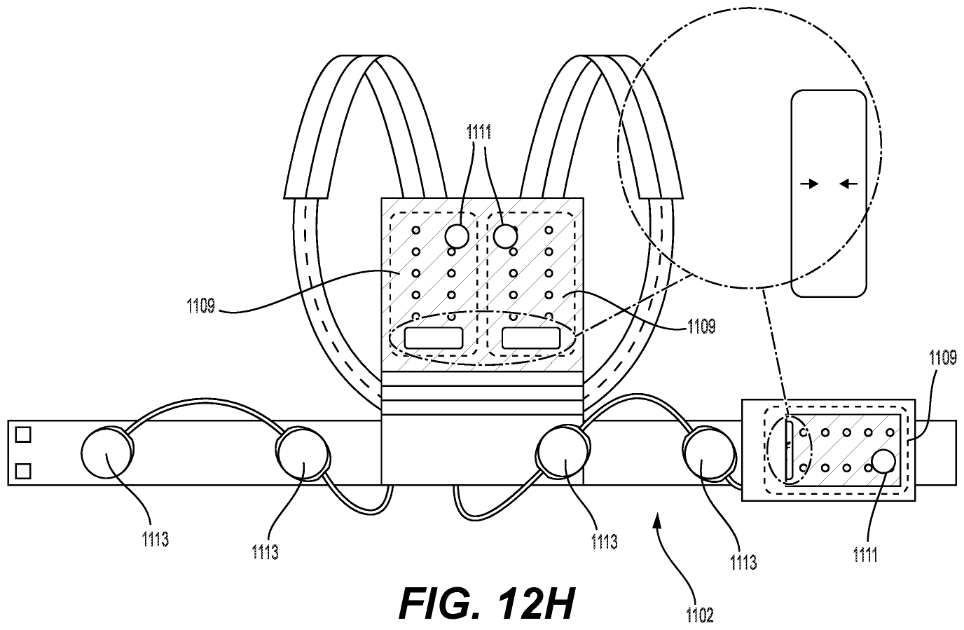

An assembly process for the garment and electrode belt illustrated in FIG. 11 is provided in FIGS. 12A-12H . FIG. 12A illustrates that the first fabric color can be configured to face a corresponding color on the therapy electrodes 1109 as they are inserted into therapy electrode pockets 1104 located in the garment 1101. As shown in FIG. 12A, the therapy electrodes 1109 can be coupled to one or more motion sensors 1111 such as anatomical placement sensors. As shown in FIG. 12A, the therapy electrodes 1109 can be slid in direction A into the therapy pockets 1104. After the therapy electrodes 1109 are engaged in their respective therapy pockets 1104 proximate where the garment would be on the back of the subject, the therapy electrodes 1109 can be secured in the therapy pockets 1104 by garment attachment features. In some embodiments the garment attachment features include snaps 1119 which are configured to engage with receiving snaps 1121 positioned below the therapy electrode pockets 1104.

FIG. 12C illustrates engagement of the vibration box 1115 of the electrode belt 1103 with a corresponding pocket in the garment 1101. As illustrated the vibration box 1115 can be positioned with a label side facing towards the garment 1101 and a connector cable 1117 facing downwards. The connector cable 1117 can be inserted under a strap 1123 positioned along the edge of the garment 1101.

As shown in FIG. 12D, the strap 1123 can include one or more fasteners or snaps for securing the vibration box 1115 to the garment 1101. For example, the strap 1123 can be moved in direction C towards a receiving snap positioned along the garment 1101.

FIG. 12E illustrates a mechanism by which the front therapy pad of the electrode belt engages with the front pocket of the garment. As shown in FIG. 12E, the therapy pad or therapy electrode 1109 can be engaged to one or more motion sensors 1111. The front therapy pad can be color coded so that the therapy pad is properly aligned with the pocket. For example, in some implementations the front therapy pad can be colored silver and a side of the receiving therapy electrode pocket can also be silver to provide a subject with a visual indicator that the silver side of the front therapy electrode is to be aligned and face the silver side of the receiving therapy electrode pocket. After the therapy electrode 1109 is fully inserted into the receiving therapy electrode pocket a snap fastener 1110 or the like can be used to securely retain the therapy electrode within the pocket.

As illustrated in FIG. 12F, after the front therapy electrode is attached to the garment 1101, additional sensors such as ECG electrodes can also be attached to the garment 1101. In the illustrated embodiment, the ECG electrode sensors 1113 can be reversibly attached to predetermined anatomical positions 1114 via Velcro® and the like. In some embodiments, the ECG electrode sensors 1113 and their respective predetermined anatomical positions 1114 can be color or icon-coded.

FIG. 12G provides an outside view of the cardiac device with the garment 1101 engaged with the electrode belt 1103 as a result of the process illustrated in FIGS. 12A-12F. The configuration illustrated in FIG. 12G shows the cardiac device assembly that faces away from the subject's body. Notably foam sides of the therapy electrodes are configured to face the back of the garment.

FIG. 12H provides an inside view of the cardiac device with the garment 1101 engaged with the electrode belt 1103 as a result of the process illustrated in FIGS. 12A-12F. The configuration illustrated in FIG. 12H shows the cardiac device assembly that face inside and towards the subject's body when worn. When properly worn, the therapy electrodes 1109 can be configured to face towards the subject's body. The described anatomical placement sensors 1111 can be used to determine the facing orientation of the therapy electrodes 1109.

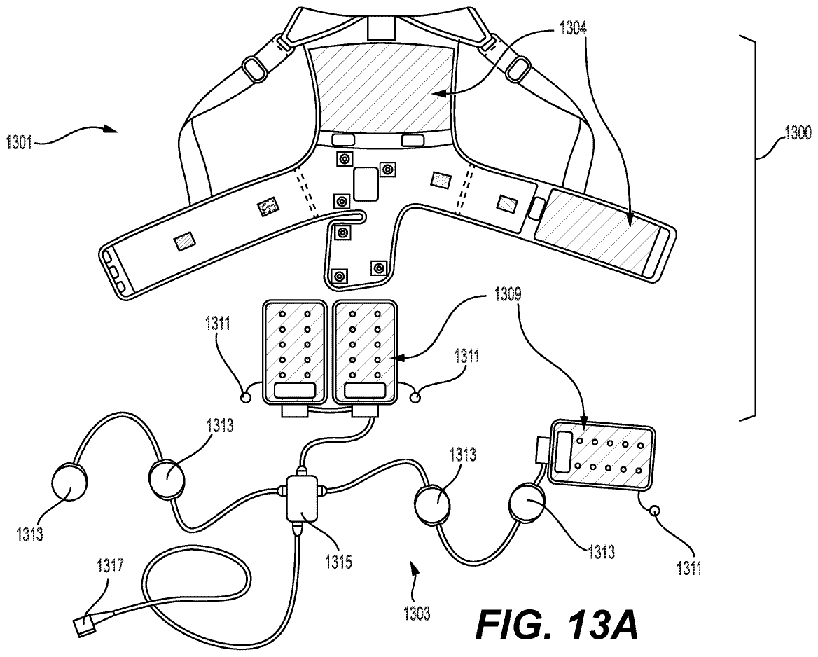

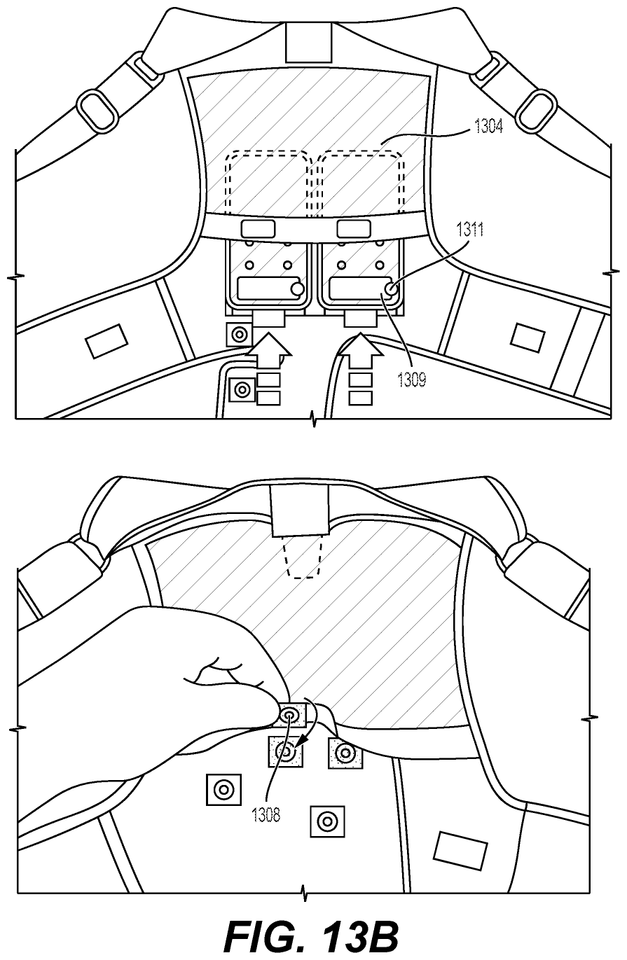

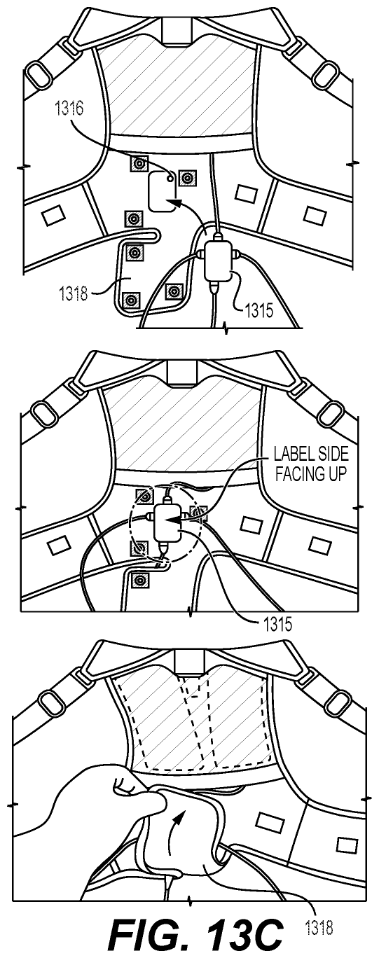

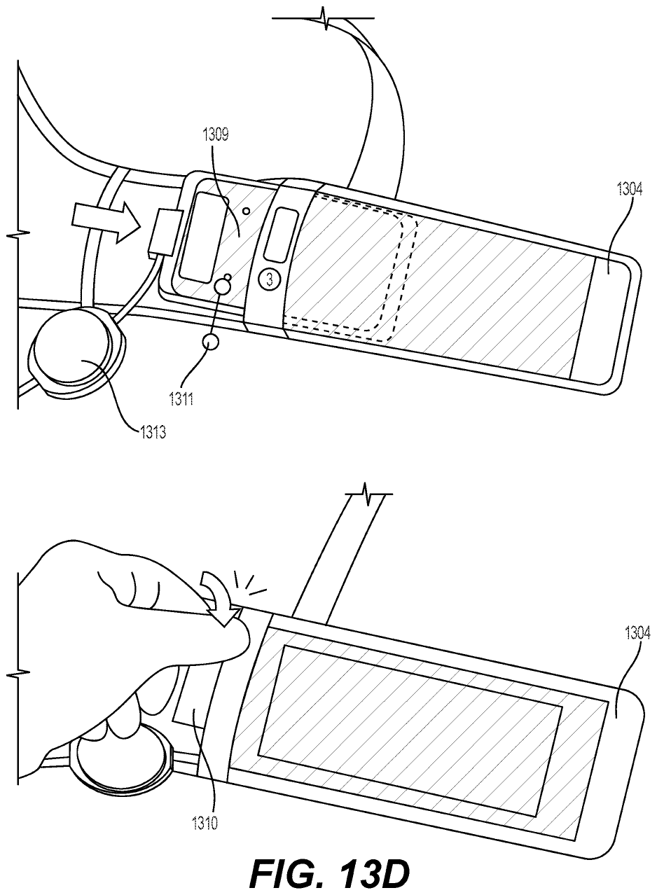

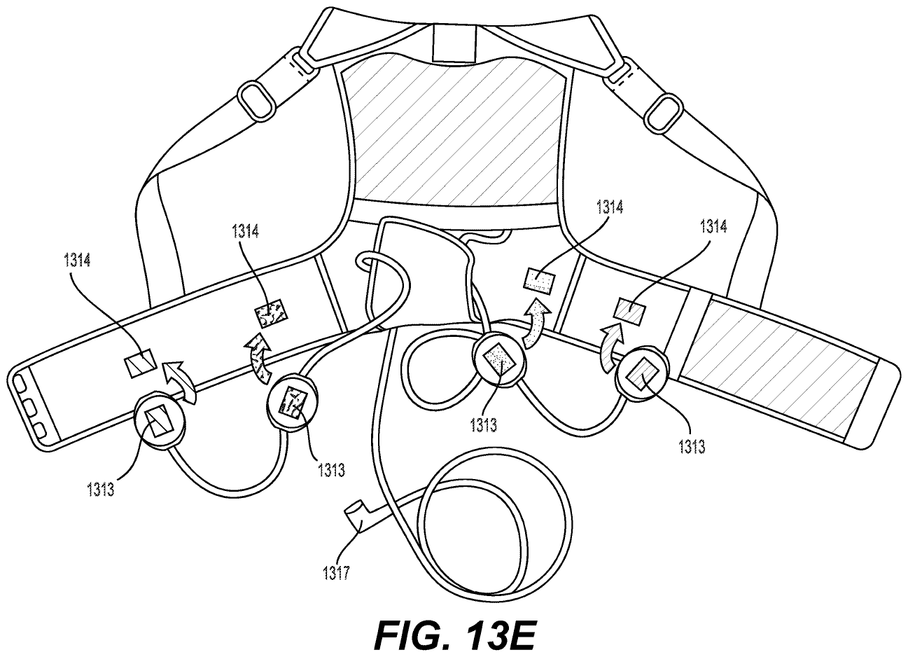

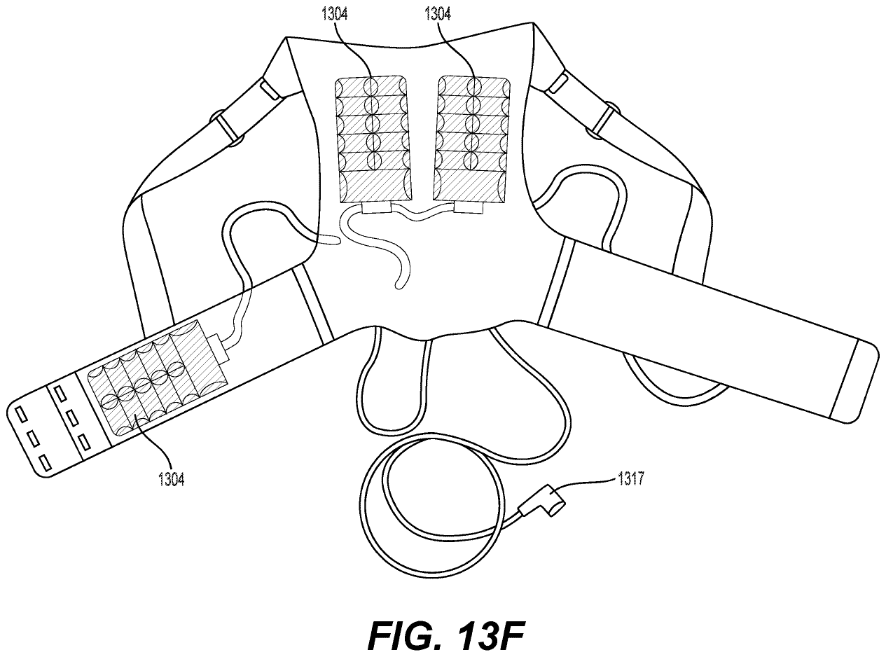

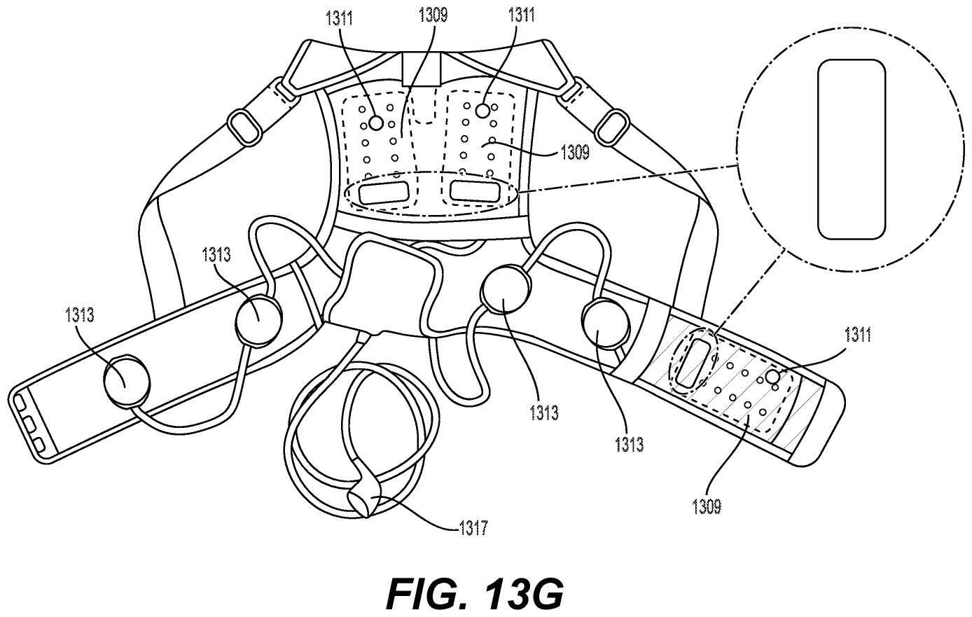

FIGS. 13A-13G provide an assembly process for another embodiment of a cardiac device including a garment and electrode belt. The cardiac device can include a wearable garment 1301 that can be associated with an electrode belt 1303. The wearable cardiac device can be configured such that the garment 1301 is configured to receive the electrode belt 1303 and form an assembly (shown in FIGS. 13F, 13G).

As shown in FIG. 13A, a garment 1301 can include a belt region for holding a therapy pad and a back region that holds two therapy pads 1304. The garment 1301 can include one or more color coordinated portions configured to guide a user to assemble the electrode belt 1303 into the garment 1301. For example, the garment 1301 can include a first fabric color around the therapy electrode pockets 1304 that is configured to receive corresponding therapy electrodes 1309 and their associated motion sensors 1311 on the electrode belt 1303. For example, at least one face of the therapy electrodes 1309 can also be of the same first fabric color. The electrode belt 1303 can include additional sensors 1313, a controller 1315 and a connector 1317, analogous to that for the implementations discussed above. The garment 1301 can also include a plurality of garment attachment features that are configured to secure one or more components of the electrode belt 1303 to the garment 1301. For example, the garment attachment features can include hook and loop fasteners, snap buttons, or seams. In the illustrated embodiment, the garment attachment features include snap buttons or snap tabs that are configured to secure the therapy electrodes 1309 in the electrode belt 1303 within their respective therapy electrode pockets 1304 when the electrode belt 1303 is assembled into the garment 1301. In some embodiments, the garment attachment features can be color-coded to provide visual guidance to a subject. In some embodiments, the fabric color for the garment attachment feature can be different from the fabric color of portions of the garment configured for receiving the electrode belt. Additional garment attachment features can be used for one or more sensors of the electrode belt 1303 such as ECG sensors 1313 composed of ECG sensing electrodes.

An assembly process for the garment and electrode belt illustrated in FIG. 13A is provided in FIGS. 13B-E. FIG. 13B illustrates that the first fabric color can be configured to face a corresponding color on the therapy electrodes 1309 as they are inserted into therapy electrode pockets 1304 located in the garment 1301. As shown in FIG. 13B, the therapy electrodes 1309 can be coupled to one or more motion sensors 1311 such as anatomical placement sensors. As shown in FIG. 13B, the therapy electrodes 1309 can be slid in direction A into the therapy pockets 1304. After the therapy electrodes 1309 are engaged in their respective therapy pockets 1304 proximate where the garment would be on the back of the subject, the therapy electrodes 1309 can be secured in the therapy pockets 1304 by garment attachment features. In some embodiments the garment attachment features include snaps 1308 which are configured to engage with receiving snaps positioned below the therapy electrode pockets 1304.

FIG. 13C illustrates engagement of the vibration box or controller 1315 of the electrode belt 1303 with a corresponding region 1316 in the garment 1301. The corresponding region 1316 can be marked with visual indicators such as numerical labeling, colored fabric, and the like. As illustrated the vibration box 1315 can be positioned with a label side facing towards the garment 1301 and a connector cable 1317 facing downwards. The connector cable 1317 can be configured to access the vibration box 1315. The vibration box 1317 can be secured to the garment 1301 by snapping a flap 1318 over the vibration box 1317 into one or more receiving fasteners or snaps.

FIG. 13D illustrates a mechanism by which the front therapy pad 1309 of the electrode belt 1303 engages with the front pocket 1304 of the garment. The therapy pad or therapy electrode 1309 can be engaged to one or more motion sensors 1311. The front therapy pad can be color coded so that the therapy pad is properly aligned with the pocket. For example, in some implementations the front therapy pad can be colored silver and a side of the receiving therapy electrode pocket can also be silver to provide a subject with a visual indicator that the silver side of the front therapy electrode is to be aligned and face the silver side of the receiving therapy electrode pocket. After the therapy electrode 1309 is fully inserted into the receiving therapy electrode pocket 1304 a snap fastener 1310 or the like can be used to securely retain the therapy electrode within the pocket.

As illustrated in FIG. 13E, after the front therapy electrode is attached to the garment 1301, additional sensors such as ECG electrodes can also be attached to the garment 1301. In the illustrated embodiment, the ECG electrode sensors 1313 can be reversibly attached to predetermined anatomical positions 1314 via Velcro® and the like. In some embodiments, the ECG electrode sensors 1313 and their respective predetermined anatomical positions 1314 can be color or icon-coded. In some embodiments, the predetermined anatomical positions 1314 can correspond with anatomical positions along the subject's body in accordance with common ECG techniques. For example, the ECG electrode sensors corresponding to the V1, V2, V3, V4, V5 and/or V6 signals. For example, the V1 electrode sensor can be positioned at the 4th intercostal space, right margin of the sternum, V2 electrode sensor can be positioned at the 4th intercostal space, left margin of the sternum, V3 electrode sensor can be positioned midway between V2 and V4, V4 electrode sensor can be positioned at the 5th intercostal space, mid-clavicular line, V5 electrode sensor can be positioned at the 5th intercostal space, anterior axillary line, and the V6 electrode sensor can be positioned at the 5th intercostal space, mid-axillary line.

FIG. 13F provides an outside view of the cardiac device with the garment 1301 engaged with the electrode belt 1303 as a result of the process illustrated in FIGS. 13B-13E. The configuration illustrated in FIG. 13F shows the cardiac device assembly that faces away from the subject's body. Notably foam sides of the therapy electrodes are configured to face the back of the garment.

FIG. 13G provides an inside view of the cardiac device with the garment 1301 engaged with the electrode belt 1303 as a result of the process illustrated in FIGS. 13B-13E. The configuration illustrated in FIG. 13G shows the cardiac device assembly that face inside and towards the subject's body when worn. When properly worn, the therapy electrodes 1309 can be configured to face towards the subject's body. The described anatomical placement sensors 1311 can be used to determine the facing orientation of the therapy electrodes 1309.

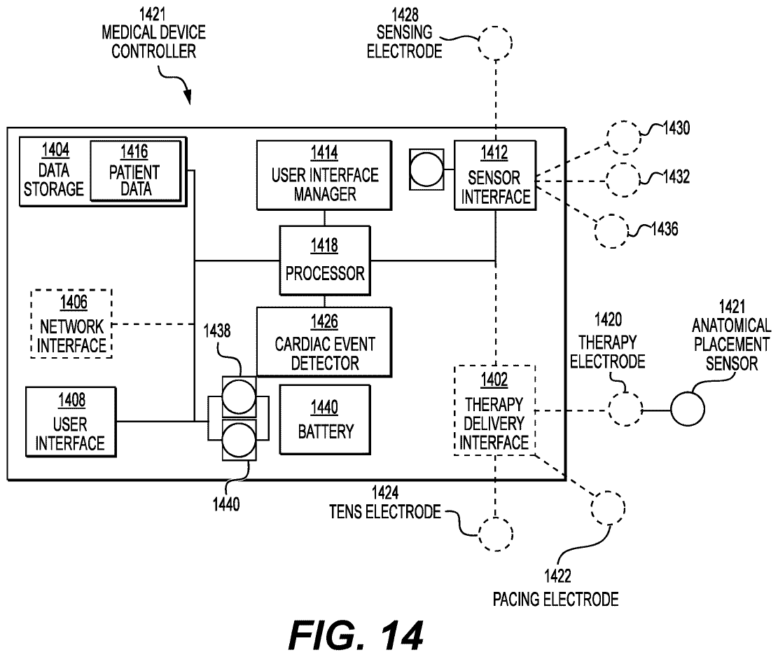

FIG. 14 provides a schematic example of a medical device controller 1421 in accordance with the disclosure herein. The controller 1421 includes at least one processor 1418, a user interface manager 1414, a sensor interface 1412, an optional therapy delivery interface 1402, data storage 1404 (which may include patient data storage 1416), an optional network interface 1406, a user interface 1408 (e.g., including the touch screen 1420), and a battery 1410. The sensor interface 1412 can be coupled to any one or combination of sensors to receive information indicative of cardiac activity. For example, the sensor interface 1412 can be coupled to one or more sensing devices including, for example, sensing electrodes 1428, contact sensors 1430, pressure sensors 1432, and accelerometers or motion sensors 1434. The therapy delivery interface 1402 (if included) may be coupled to one or more electrodes that provide therapy to the patient including, for example, one or more therapy electrodes 1420, pacing electrodes 1422, and/or TENS electrodes 1424. In some embodiments, the therapy electrodes 1420 can be coupled to one or more anatomical placement sensors 1421 including accelerometers, gyroscopes, and/or inertial motion units. The sensor interface 1412 and the therapy delivery interface 1402 may implement a variety of coupling and communication techniques for facilitating the exchange of data between the sensors and/or therapy delivery devices and the controller 1420.

In some examples, the network interface 1406 can facilitate the communication of information between the controller 1420 and one or more other devices or entities over a communications network. For example, the network interface 1406 may be configured to communicate with a server (e.g., a remote server) where a caregiver can access information related to the patient. In some embodiments, network interface 1406 may facilitate communication between the medical device controller 1420 and a base station associated (e.g., paired) with the medical device controller.

In some examples, the medical device controller includes a cardiac event detector 1426 to monitor the cardiac activity of the patient and identify cardiac events experienced by the patient based on received cardiac signals. In other examples, cardiac event detection can be performed using algorithms for analyzing patient ECG signals obtained from the sensing electrodes 1428. Additionally, the cardiac event detector 1426 can access patient templates (e.g., which may be stored in the data storage 1404 as patient data 1416) that can assist the cardiac event detector 1426 in identifying cardiac events experienced by the particular patient (e.g., by performing template matching algorithms).

The at least one processor 1418 can perform a series of instructions that control the operation of the other components of the controller 1420. In some examples, the user interface manager 1414 is implemented as a software component that is stored in the data storage 1404 and executed by the at least one processor 1418 to control, for example, the user interface component 1408. The user interface manager 1414 can control various outputs or output components and/or devices of the medical device controller 1420 to communicate with external entities consist with various acts and/or display screens described herein. For example, such outputs or output components and/or devices can include speakers, tactile and/or vibration output elements, visual indicators, monitors, displays, LCD screens, LEDs, Braille output elements, and the like.

In some implementations, the user interface 1408 can include one or more interfaces for communicating and/or interacting with different external entities. For example, such interfaces can include a caregiver interface for communicating and/or interacting with a caregiver (e.g., a nurse, a physician, a physician's aide, or other such individual or entity), a patient interface for a patient, a patient service representative interface for a patient service representative, or a service interface for a service technician, among others. For example, the one or more interfaces can be displayed on a same physical display and/or touchscreen. In some cases, the external entities may be assigned separate security credentials that may be provided before access is granted to the corresponding interface. In some examples, the one or more interfaces can be displayed on different physical displays and/or touchscreens. For example, a caregiver interface may be displayed on a first display, and a patient interface may be displayed on a second, different display.

For example, the user interface manager 1414 may cause the user interface 1408 to switch from a first one of the one or more interfaces to a second one of the one or more interfaces depending on a current device function or operation. As an example, the user interface 1408 can display “Call Caregiver” to the patient via a patient interface when a device related event is detected. When the caregiver arrives, he or she may provide his or her security credentials and access a caregiver interface for addressing the device related event.

In some examples, the medical device can be a patient monitoring device, which can be configured to monitor one or more of a patient's physiological parameters without an accompanying treatment component. For example, a patient monitor may include a cardiac monitor for monitoring a patient's cardiac information. Such cardiac information can include, without limitation, heart rate, ECG data, heart sounds data from an acoustic sensor, and other cardiac data. In addition to cardiac monitoring, the patient monitor may perform monitoring of other relevant patient parameters, including glucose levels, blood oxygen levels, lung fluids, lung sounds, and blood pressure.

An example cardiac monitoring medical device (e.g., a cardiac monitor) may be similar to wearable medical device described herein and omit, for example, the therapy electrodes 1420 and/or the therapy delivery interface 1402. In some implementations, the cardiac monitor is capable of and designed to be worn by a patient who is at risk of developing cardiac problems, but who does not yet meet criteria to be outfitted with a medical device that includes a treatment component (e.g., a defibrillator). Thus, the cardiac monitor can be prescribed so that continuous and/or event-based data can be sent from the cardiac monitor to a remote server. A caregiver can access the data from the remote server and determine whether the patient is experiencing or has experienced a cardiac problem. In some implementations, after determining that the patient is experiencing a cardiac problem, the caregiver can instruct the patient to begin wearing a medical device with treatment capabilities.

In some implementations, the patient can interact with the user interface 1408 to identify a patient symptom. The user interface 1408 can include a drop down menu or check list that allows the patient to select a particular symptom from a list of alternatives. Options for patient systems can include one or more of: feeling a skipped beat, shortness of breath, light headedness, racing heart rate, fatigue, fainting, chest discomfort, weakness, dizziness, and/or giddiness. In addition, the patient can select a level of activity (e.g., light activity, moderate activity, rigorous activity, etc.) that he or she was performing when the symptom occurred. In some implementations, in response to the selection by the patient, the cardiac event detector 1426 can cause a portion of patient physiological information (e.g., in the form of a cardiac signal) to be captured for a length of time that is based on when the symptom was experienced. For example, the cardiac event detector 1426 can cause a portion of an ECG signal of the patient to be captured. The portion of the ECG signal is sometimes referred to herein as an ECG strip. In some implementations, the cardiac monitor can continuously record ECG data, and at the same time also identify and record one or more ECG strips relating to one or more events of interest (e.g., patient-reported symptoms, events detected by the cardiac event detector 1426, etc.). As such, if a caregiver wishes to view ECG data for a period of time prior to or after the recorded ECG strip relating to an event of interest, such data is available for review from the continuously-recorded ECG data.

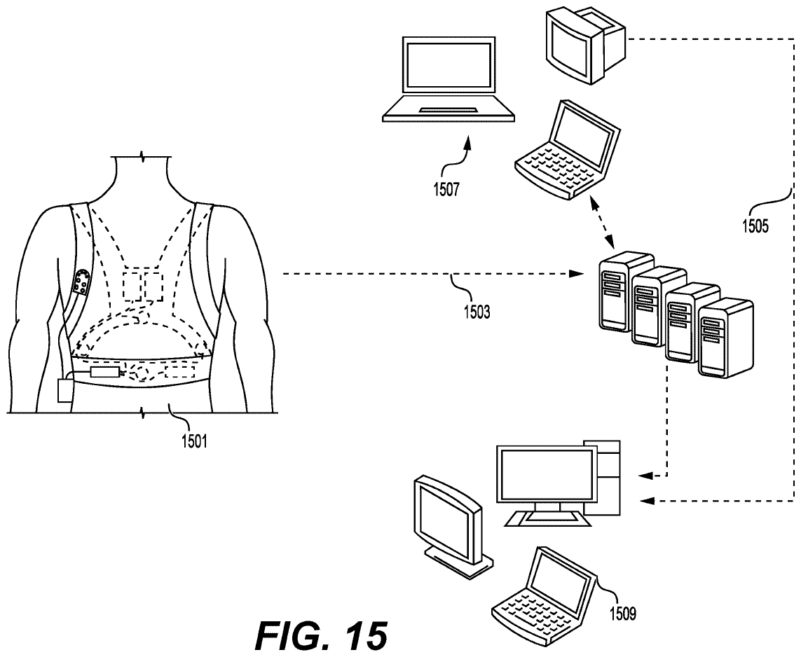

FIG. 15 illustrates a data transmission system for use in connection with the cardiac wearable device 1501. As shown, the cardiac wearable device 1501 can be communicatively coupled to a server 1505 by way of a network 1503. Data can be stored by the server 1505 or transmitted to user devices 1507, 1509 and the like. User devices can include devices used by the subject wearing the cardiac wearable device 1501 and/or medical professionals and the like. As shown, the system includes a cardiac wearable device 1501, user devices 1507, 1509, a remote server 1505 (e.g., accessible to a remote technician via a workstation), a network connection 1503 and related communication network. The medical device 1501 exchanges information with the remote computing device or user device 1507, 1509 via the network 1503. Similarly, the remote computing device user devices 1507, 1509 may exchange information with the remote server 1505 via the network 1503. In at least one example, the remote server 1505 can be configured to deploy or download one or more components (e.g., programs or “apps”) to the remote computing device or user devices 1507, 1509 that cause the remote computing device to transmit particular commands to the cardiac wearable device 1501. In some implementations, the remote server 1505 can be configured to deploy or download the one or more components to the cardiac wearable device 1501 (e.g., containing the controller).

In various examples, the remote computing device or user device 1507, 1509 is implemented using any of a variety of programmable devices (e.g., a device with data storage and at least one processor in data communication with the data storage). In some examples, the remote computing device 1507 includes a plurality of interfaces, one or more processors, and a data storage device coupled to one another via a communication mechanism, such as a bus. In these examples, the remote computing device 1507 also includes a battery to power the device and may include one or more antennas. The plurality of interfaces in the remote computing device 1507 include a user interface, a network interface configured to communicate with the network 1503 and a medical device interface configured to exchange information with the medical device or cardiac wearable device 1501. This information may include one or more limited functionality commands. Particular examples of the remote computing device 1507, 1509 include medical devices wearable devices, smart phones, tablet computers, and laptop computers. Wearable devices that may serve as the remote computing device 1507, 1509 include various garments with integrated technologies, watches, anklets, necklaces, belt buckles, and glasses.

In some examples, the remote computing device can establish an authenticated and secure connection over the network 1503 via another computing device (e.g., a desktop workstation, laptop workstation, tablet or other such device). For example, the user may cause the remote computing device 1507, 1509 (e.g., a smart phone) to establish a wired (e.g., USB connection) or wireless connection (e.g., BLUETOOTH connection) with the other computing device to connect to the remote server 1505.

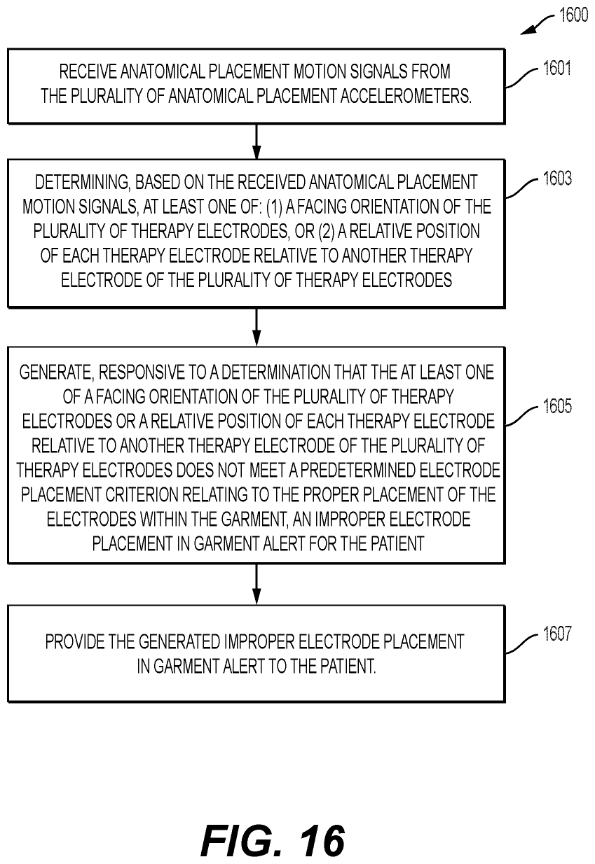

FIG. 16 illustrates a process or method for electrode placement verification. The process includes verifying facing orientation of electrodes such as treatment or monitoring electrodes in a garment worn by an ambulatory patient. As described above, a subject may be required to assemble one or more therapy electrodes into the wearable garment without medical supervision. Proper orientation and/or placement of the therapy electrodes within the wearable garment is required for providing effective therapeutic treatment. The process illustrated in FIG. 16 describes a process for determining whether electrodes are facing outwards (i.e., away from a patient) or inwards (i.e., towards a patient) when the therapy electrodes are assembled into the wearable garment. The described process can also provide an indication if electrodes are properly placed in the wearable garment by comparing their placement in relation to other electrodes in the garment.

As illustrated in FIG. 16, the method may include the steps of: receiving anatomical placement motion signals from a plurality of anatomical placement accelerometers 1601, determining, based on the received anatomical placement motion signals, at least one of: (1) a facing orientation of the plurality of therapy electrodes, or (2) a relative position of each therapy electrode relative to another therapy electrode of the plurality of therapy electrodes 1603, generating, responsive to a determination that the at least one of a facing orientation of the plurality of therapy electrodes or a relative position of each therapy electrode relative to another therapy electrode of the plurality of therapy electrodes does not meet a predetermined electrode placement criterion relating to the proper placement of the electrodes within the garment, an improper electrode placement in garment alert for the patient 1605, and providing the generated improper electrode placement in garment alert to the patient 1607.

In some embodiments, a wearable cardiac device can be configured to execute the steps of the process illustrated in FIG. 16. For example, the wearable cardiac device can be configured to verify the facing orientation of electrodes in a garment worn by an ambulatory patient. In some embodiments, the garment can include one or more therapy electrode pockets configured to align with one or more predetermined anatomical locations on a patient. The wearable cardiac device can also include a plurality of ECG sensing electrodes that are configured to sense ECG signals of a patient and to be disposed at one or more predetermined ECG sensing locations within the garment. The wearable cardiac device can also include a plurality of therapy electrodes that are configured to deliver one or more therapeutic shocks to the patient. In some implementations each therapy electrode of the plurality of therapy electrodes can be configured to be disposed within a therapy electrode pocket of the one or more therapy electrode pockets of the garment, as described herein. The wearable cardiac device can also include one or more motion sensors such as anatomical placement accelerometers, anatomical placement gyroscopes and/or anatomical placement inertial motion units. The motions sensors such as the anatomical placement accelerometer can be integrated with a corresponding therapy electrode of the plurality of therapy electrodes. The anatomical placement motion sensors can be configured to generate anatomical placement motion signal for the corresponding therapy electrode.

In some implementations, the wearable cardiac device can include one or more physiological sensors separate from the plurality of ECG sensing electrodes, the plurality of therapy electrodes and the plurality of anatomical placement accelerometers. The physiological sensors can be configured to sense physiological signals from the ambulatory patient. For example, the physiological sensors can include cardiovibrational sensors for sensing heart sounds, RF antenna and circuitry for determining lung fluid metrics and/or phytoplethysmography (PPG) sensors for determining blood oxygenation, and the like.

In some implementations, the therapy electrodes are configured to deliver one or more therapeutic shocks to the patient responsive to detection of a cardiac arrhythmia based on ECG signals sensed from the plurality of ECG sensing electrodes. In some implementations, each of the one or more therapy electrodes can be coupled to a respective ECG sensing electrode of the one or more ECG sensing electrodes to form a multifunctional electrode.

In some implementations the garment can include one or more garment attachment features for removably coupling the plurality of ECG sensing electrodes, the plurality of therapy electrodes and/or the plurality of anatomical placement accelerometers to the garment. Garment attachment features comprise clasps, hook and loop fasteners, button and hole fasteners, snap buttons, and the like. In some implementations, the garment can be permanently coupled to the ECG sensing electrodes by using one or more seams in the garment.

In some implementations, a controller in electrical communication with the plurality of electrodes and the plurality of motion sensors including anatomical placement accelerometers can be configured to execute the method illustrated in FIG. 16.

As illustrated in FIG. 16, the controller can receive anatomical placement motion signals from a plurality of motion sensors analogous to motion sensors 201, 515, 613, 713, 1111, 1311, and 1434 described herein. Motion sensors can be coupled to therapy electrodes and include anatomical placement sensors which may include anatomical placement accelerometers, anatomical placement inertial motion units, anatomical placement gyroscopes and the like.

The anatomical placement sensors can include a circuit having at least one of a gyroscope, accelerometer, magnetometer, and/or inertial motion unit. The motion sensors can include one or more anatomical placement gyroscopes configured to generate at least one anatomical placement motion signal indicating at least one of a rotation direction, rotation angle and/or vibration. The motion sensors can include at least one anatomical placement accelerometer which is configured to generate at least one anatomical placement motion signal indicating linear acceleration in along an x-axis, y-axis, or z-axis. In some embodiments, the motion sensors can include an anatomical placement inertial motion unit that is configured to generate at least one anatomical placement motion signal indicating at least one of a linear acceleration in an x-axis, a linear acceleration in an y-axis, a linear acceleration in a z-axis, a rotational acceleration around an x-axis, a rotational acceleration around a y-axis and/or a rotational acceleration around a z-axis. Anatomical placement motion signals can include signals indicating placement, velocity, and/or acceleration in one or more axis generated by the anatomical placement sensors. The anatomical placement motion signals can provide data indicative of motion associated with the associated therapy electrode.

As indicated in FIG. 16, based on the received anatomical placement motion signals, the controller can be configured to determine a facing orientation of the plurality of therapy electrodes 1603. Facing orientation can refer to the orientation and positioning of a therapy electrode within a corresponding therapy electrode pocket and/or against the patient's skin. The therapy electrodes can be configured to have directionality, in that a particular face or side of the therapy electrode can be configured to face a subject to apply the therapeutic treatment to the patient. Accordingly, when the therapy electrodes of the belt are engaged with the garment and inserted into their respective therapy electrode pockets, the therapy electrodes are required to be facing the proper orientation such that therapeutic treatments can be applied properly.

As shown in FIG. 16, the controller can also be configured to determine a relative position of each therapy electrode relative to another therapy electrode of the plurality of therapy electrodes 1603 based on the received anatomical placement motion signals. For example, in some implementations therapy electrodes can be configured to be positioned at different locations in a garment for a wearable cardiac device. In a garment worn by an ambulatory subject, the distance between the therapy electrodes is maintained when the electrodes are properly situated in the garment and the subject is moving. Additionally, orientation or rotational information from signals obtained from a plurality of therapy electrodes remain consistent if the therapy electrodes are appropriately positioned within the garment. Accordingly, by detecting inconsistencies or variations in distance between various therapy electrodes based on the received anatomical placement motion signals, the relative position of each therapy electrode relative to another therapy electrode can be determined and used to determine whether an improper electrode placement in garment alert is appropriate.

Various techniques for determining the facing orientation and/or relative position of each therapy electrode relative to another therapy electrode of the plurality of therapy electrodes can be used.

For example, in some embodiments determining at least one of the facing orientation of the plurality of therapy electrodes, or the relative position of each therapy electrode relative to another therapy electrode of the plurality of therapy electrodes further can include processing or pre-processing the received at least one anatomical placement motion signal by applying a filter to the received at least one anatomical placement motion signal, and/or applying an alignment in time to the received at least one anatomical placement motion signal. Additionally, the received at least one anatomical placement motion signal can be filtered to remove a contribution due to patient breathing from the anatomical placement motion signal.

As illustrated in FIG. 16, the controller can generate an improper electrode placement in garment alert for the patient 1605. The improper electrode placement in garment alert can be responsive to a determination that the at least one of a facing orientation of the plurality of therapy electrodes or a relative position of each therapy electrode relative to another therapy electrode of the plurality of therapy electrodes does not meet a predetermined electrode placement criterion relating to the proper placement of the electrodes within the garment.

The predetermined electrode placement criterion can include at least one of a prescribed location, prescribed orientation, and/or prescribed facing orientation. The prescribed location may be indicative of a distance from a therapy electrode to a central origin of the ambulatory patient. The prescribed orientation can be indicative of an angle formed between a side of the therapy electrode and a central origin of the ambulatory patient. The prescribed facing orientation can include a direction as to which therapeutic shock elements of the therapy electrodes are prescribed to be facing. Each therapy electrode in the garment and therapy electrode assembly can be assessed to determine whether their respective orientation, location, and/or facing orientation corresponds to the prescribed location, prescribed orientation and/or prescribed facing orientation of the predetermined electrode placement criterion. A comparison of at least one of an angle, a direction, or a magnitude of the therapy electrode can be made to the remaining therapy electrodes to assist in determining whether the predetermined electrode placement criterion is met.

In some implementations, the predetermined electrode placement criterion can be based on the location and orientation of previous therapy electrodes or historical electrode placement. In some implementations, the predetermined electrode placement criterion can be based on predefined electrode placement conditions or conditions set by a medical practitioner or a device manufacturer or the like. In some implementations, the predetermined electrode placement criterion can be based on electrode placement information obtained from a plurality of subjects.

In some implementations, the predetermined electrode placement criterion can specify a facing orientation for the plurality of therapy electrodes, which indicates the direction the therapeutic shock elements of the plurality of therapy electrodes are facing. When one or more of the therapy electrodes do not have the same facing orientation as the predetermined electrode placement criterion an improper electrode placement in garment alert can be generated. In such a situation, the therapy electrode may have been inserted improperly into a pocket such that the shock applying elements face outward, away from the patient rather than towards the patient.

In some implementations, the relative position of each therapy electrode relative to another therapy electrode of the plurality of therapy electrodes can be expressed as an inter-therapy electrode distance and inter-therapy electrode angle. The inter-therapy electrode distance and inter-therapy electrode angle can be analyzed over time to determine if there is drift or inconsistent movement between two or more of the plurality of therapy electrodes. In this manner, therapy electrodes that are behaving anomalously with respect to other therapy electrodes can be identified. Determining at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment can be based on a comparison with electrode placements associated with a remainder of the plurality of therapy electrodes. Further, the comparison can include a comparison of at least one of an angle, a direction, or a magnitude.

In some embodiments, at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in an angle of the associated plurality of therapy electrodes and an angle of the predetermined electrode placement criterion is greater than a suitable percentage of the angle of the predetermined electrode placement criterion. The suitable percentage can be less than 10 percent, e.g., 9 percent, 8, percent, 7 percent, 6 percent, 5 percent, 4 percent, 3 percent, 2 percent, 1 percent or any such value in between.

In some implementations, at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a direction of the associated at least one of the plurality of electrodes and a direction of the predetermined electrode placement criterion is greater than a suitable percentage of the direction of the placement condition. The suitable percentage can be less than 10 percent, e.g., 9 percent, 8, percent, 7 percent, 6 percent, 5 percent, 4 percent, 3 percent, 2 percent, 1 percent or any such value in between.

In some implementations at least one of the facing orientation of the plurality of therapy electrodes or the relative position of each therapy electrode relative to another therapy electrode of the plurality of therapy electrodes does not meet a predetermined electrode placement criterion relating to proper placement of the electrodes within the garment when a difference in a magnitude of the associated at least one of the plurality of electrodes and a magnitude of the predetermined electrode placement criterion is greater than a suitable percentage of the magnitude of the placement condition. The suitable percentage can be less than 10 percent, e.g., 9 percent, 8, percent, 7 percent, 6 percent, 5 percent, 4 percent, 3 percent, 2 percent, 1 percent or any such value in between.

In some implementations, the plurality of therapy electrodes can form a treatment vector for the ambulatory patient. For example, the therapy electrodes can be configured to sequentially shock the patient.

In some implementations, determining at least one of the facing orientation of the plurality of therapy electrodes, or the relative position of each therapy electrode relative to another therapy electrode of the plurality of therapy electrodes can include processing the received at least one anatomical placement motion signal. Processing the received anatomical placement motion signal can include determining a four-dimensional representation of the plurality of therapy electrodes and determining a therapy electrode orientation based on the determined four-dimensional representation.

In some implementations, the controller can be further configured to identify the at least one therapy electrode among the plurality of therapy electrodes that has a corresponding anatomical placement accelerometer that is indicating that the therapy electrode does not meet the anatomical placement condition. In some embodiments, the identified therapy electrode can be disabled from delivering one or more therapeutic shocks since it does not meet the predetermined conditions. Alternatively, the identified therapy electrode can be provided to the subject via an alert.

Additionally, as shown in FIG. 16, the controller can provide the generated improper electrode placement in garment alert to the patient 1607. The improper electrode placement in garment alert can include an audible indicator, visual indicator, or vibration. The improper electrode placement in garment alert can also be provided in a graphical user interface in electrical communication with the controller. In some embodiments, the improper electrode placement in garment alert can notify the subject which of the therapy electrodes or motions sensors is triggering the alert.

In some embodiments, the alert can provide a subject with a direction as to corrective action to be taken to fix the electrode placement so that an improper electrode placement in garment alert is no longer issued. Accordingly, in some implementations the controller may monitor the cardiac device and/or subject to determine whether a corrective action is taken, and if a corrective action has not been taken, transmitting the generated improper electrode placement in garment alert to a second party device in order to escalate the alert. The second party device may belong to a medical professional, medical device company, or other services.

In some embodiments, the improper electrode placement in garment alert can be used to determine electrode fall-off, e.g., when an electrode is making improper or insufficient contact with the patient's skin. In some embodiments, the anatomical placement motion signal can be used to determine such electrode fall-off.

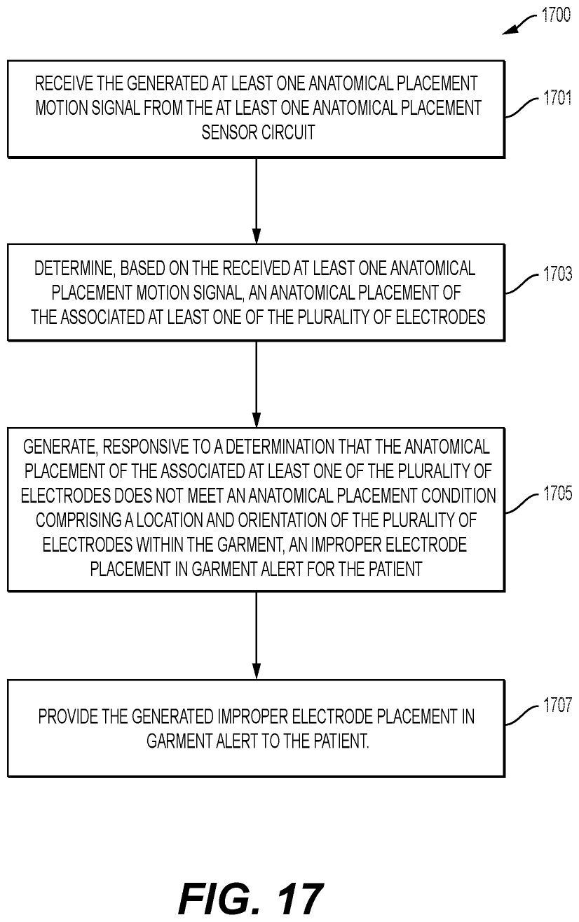

FIG. 17 illustrates a process or method for electrode placement verification. The process includes verifying anatomical placement of electrodes on an ambulatory patient. The method can include the steps of: receiving at least one anatomical placement motion signal generated from the at least one anatomical placement sensor circuit 1701, determining an anatomical placement of the associated at least one of the plurality of electrodes based on the received at least one anatomical placement motion signal 1703, and generating, responsive to a determination that the anatomical placement of the associated at least one of the plurality of electrodes does not meet an anatomical placement condition comprising a location and orientation of the plurality of electrodes within the garment, an improper electrode placement in garment alert for the patient 1705, and providing the generated improper electrode placement in garment alert to the patient.

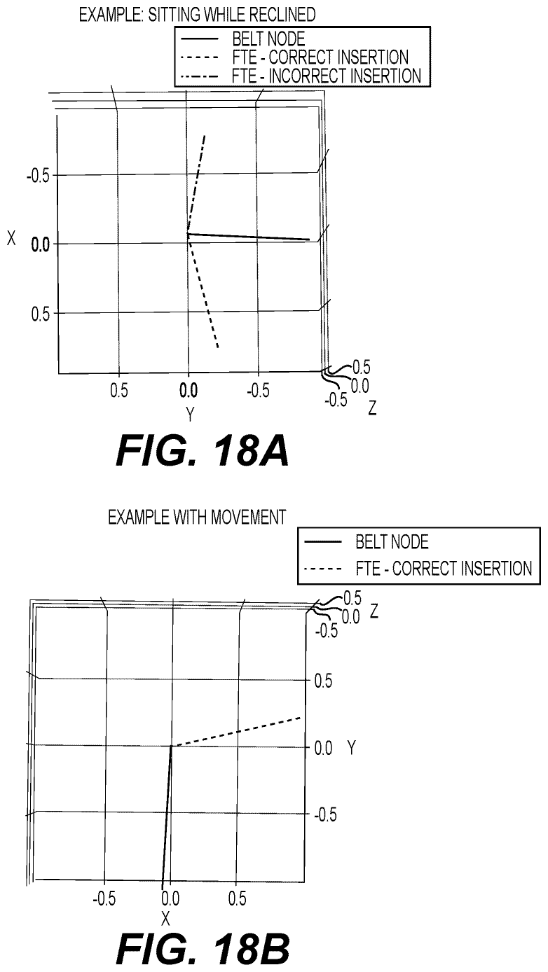

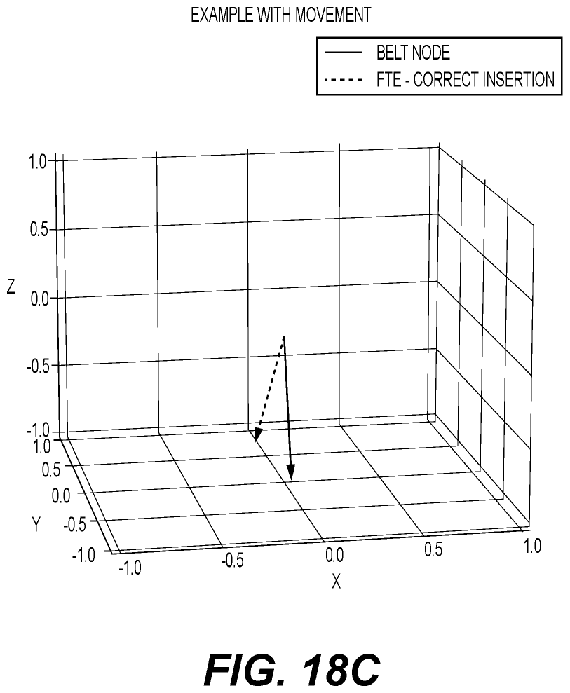

FIGS. 18A-18C illustrate examples of therapy electrode placement, facing direction, and/or orientation as determined based on an anatomical placement motion signal. As discussed herein the anatomical placement motion signal can be generated by the anatomical placement sensor circuit including at least one of an anatomical placement accelerometer, anatomical placement gyroscope, and/or an anatomical placement inertial motion unit. In particular, FIGS. 18A-18C illustrate data generated from anatomical placement sensors and provided as anatomical placement motion signals. The displayed anatomical placement motion signals can be represented as a vector including a magnitude and direction.

FIG. 18A provides an example where an ambulatory subject is sitting while reclined. The anatomical placement motion signal from three therapy electrodes is displayed in a three dimensional representation. The three therapy electrodes include the therapy electrode configured to be worn approximate the belt, along with two therapy electrodes, one of which is properly placed and another of which is improperly placed. Because the vector representation of the two therapy electrodes displays magnitudes in different directions improper electrode placement in the garment for one of the two therapy electrodes is implicated. The angle relationship between the therapy electrode at the belt and the front therapy electrode can change from “leading” to “lagging” in the X-Y plane, indicating an incorrectly installed or flipped therapy electrode having an incorrect facing orientation.

FIG. 18B provides an example where an ambulatory subject is moving. The anatomical placement motion signal from two therapy electrodes is displayed in a three dimensional representation. The two therapy electrodes include the belt therapy electrode and one additional therapy electrode. The two therapy electrodes illustrate a substantially ninety-degree angle representing their different orientations and similar magnitudes indicating that the electrodes are moving in substantially similar direction. Accordingly, it can be determined that the electrodes are properly placed in the garment.

FIG. 18C provides an example where an ambulatory subject is moving, displaying the anatomical placement motion signal from two therapy electrodes. The two therapy electrodes include the belt therapy electrode and one additional therapy electrode. The two therapy electrodes illustrate a substantially ninety-degree angle representing their different orientations and similar magnitudes indicating that the electrodes are moving in substantially similar direction. Accordingly, it can be determined that the electrodes are properly placed in the garment.

In some embodiments, the absolute vector magnitudes for the therapy electrodes as well as the relative vector magnitudes between therapy electrodes can be determined from the anatomical placement motion signals.

In some embodiments, the anatomical placement motion signals can be determined by anatomical placement accelerometers used in combination with an anatomical placement gyroscope. Alternatively, the anatomical placement motion signals can be generated by an anatomical placement motion inertial unit. The resulting anatomical placement motion signals can provide data regarding rolling motions experienced by the therapy electrodes and their corresponding anatomical placement motion sensors.

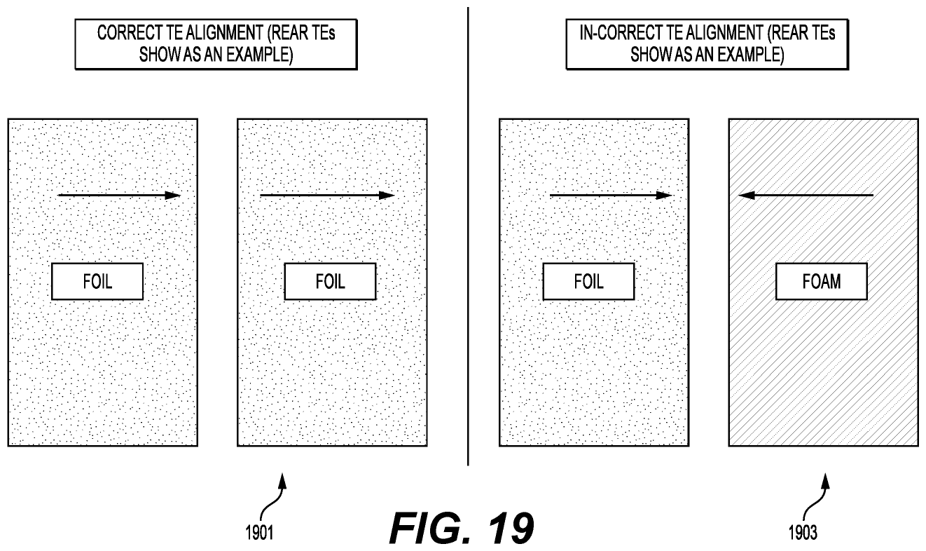

FIG. 19 illustrates the expected anatomical placement motion signals represented as a vector including a magnitude and direction for two rear therapy electrodes. As illustrated in FIG. 19 in properly placed therapy electrodes 1901, the anatomical placement motion signals may have the same orientation and/or directionality. As illustrated in FIG. 19 in improperly placed therapy electrodes 1903, the anatomical placement motion signals may have differing orientation and/or directionality. As shown in FIG. 19, improperly placed therapy electrodes 1903 may produce anatomical placement signals that indicate when a therapy electrode is flipped. For example, when a therapy electrode is flipped, the direction indicated by the vector representation of the anatomical placement motion signal may be in an opposite direction to the vector representation of the anatomical placement motion signal of the remaining electrodes.

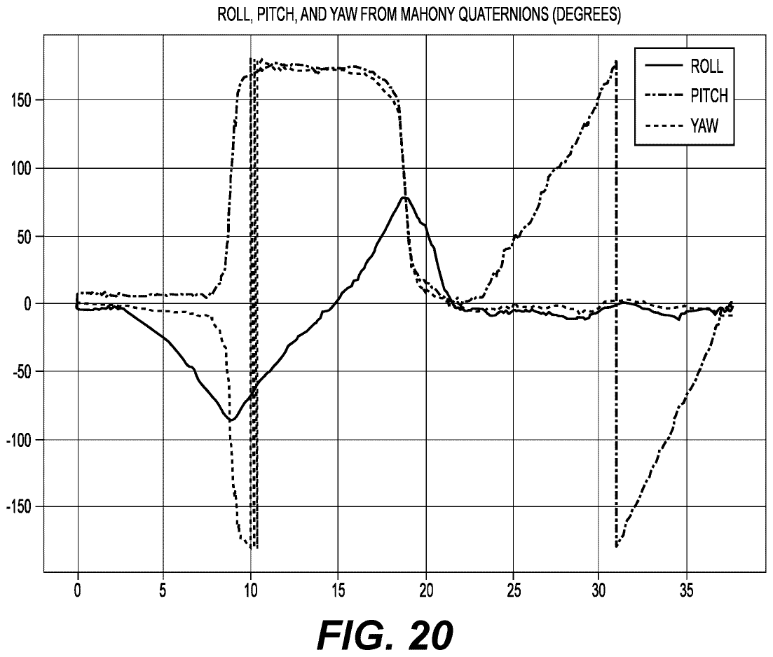

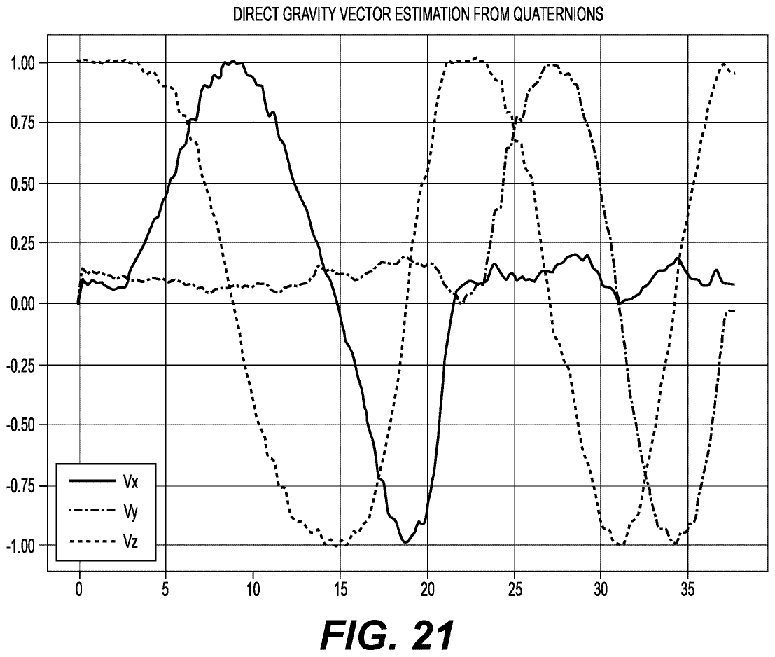

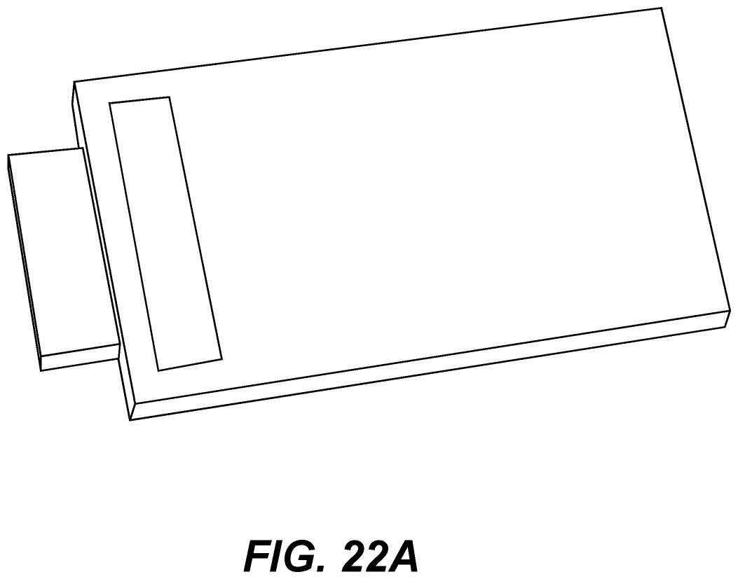

FIGS. 20 and 21 provide graphical illustrations of an output for a process for obtaining motion information from a motion sensor such as the anatomical placement accelerometer, anatomical placement gyroscope, and/or the anatomical placement inertial motion unit. In the illustrated embodiment, data from an anatomical placement inertial motion unit is filtered and used to determine four-dimensional representations of the corresponding therapy electrodes. From the four-dimensional representation, in some implementations, a gravitational impact can be determined. Using the determined gravitational impact, a representation of the anatomical placement inertial motion unit in three dimensions over time can be determined. Illustrations of the three-dimensional data from an anatomical placement inertial motion sensor is shown in FIGS. 20-21.

It is envisioned that various filters can be applied to raw data generated by the anatomical placement motion sensors. For example, filters for arial vehicle altitude estimation and/or robotics can be adapted for use with the anatomical placement motion sensors discussed herein.

The filters can be applied to inertial motion units that generate accelerometer data, gyroscope data and/or magnetometer data. The resulting filtered data can be used to generate a four-dimensional representation such as a “quaternions” with a closed-loop estimator. By using a four-dimensional representation inaccuracies due to the application of basic trigonometry can be avoided. Therapy electrode orientations can then be determined from the four-dimensional representations by estimating a gravity vector from the quaternions and converting the quaternion to Euler angles (i.e., roll, pitch and yaw). The “up” vector for each therapy electrode can then be determined by applying Rodrigues' rotation formula to the determined Euler angles.

In some implementations a Mahony orientation filter can be used. For example, such a filter is configured to calculate an orientation or one of more therapy electrodes in a short period of time by 3-axis of accelerometer, 3-axis of gyroscope, and 3-axis of magnetometer. The filter using quaternion as orientation representation to describe the therapy electrode orientation in 3-dimensions due to quaternion can avoid a singularity of Euler angle (e.g., gimbal lock).

In particular, FIG. 20 provides direct vector estimation of gravity from the quaternions determined from raw data on which a Mahony orientation filter is applied.

FIG. 21 provides a direct vector estimation of gravity from Euler angles.

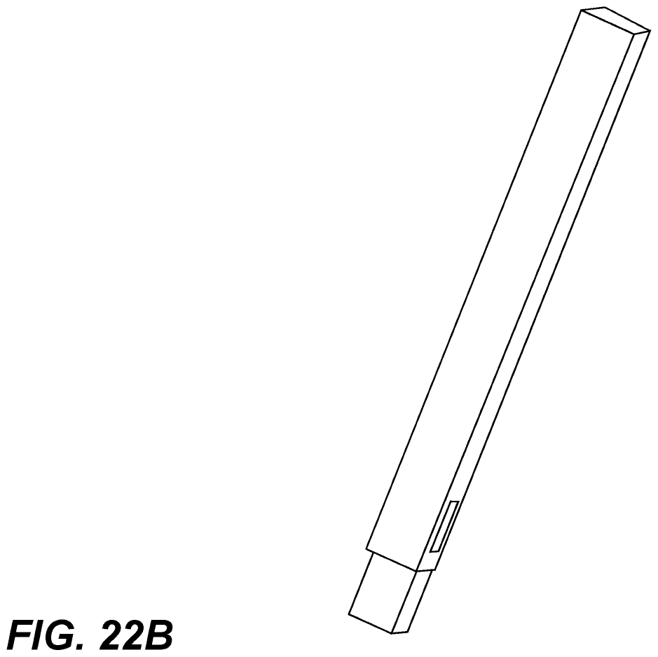

FIGS. 22A and 22B illustrate modeling of therapy electrodes based on the anatomical placement motion data and the techniques discussed herein. As illustrated in FIGS. 22A and 22B, a graphical user interface can be configured to display an image of a therapy electrode or visualization and it's movement over time based on the anatomical placement motion data. The visualization may demonstrate therapy electrode flipping or reversal in the direction that the therapy electrodes are configured to face. Therapy electrode flipping can result in generation of an improper electrode placement in garment alert.

FIG. 22A provides a first view of a therapy electrode rendered by the visualization process described herein, where the side determining the facing orientation (i.e., the side of the therapy electrode configured to apply a therapeutic shock) is shown.

FIG. 22B provides a side view of a therapy electrode rendered by the visualization process described herein.

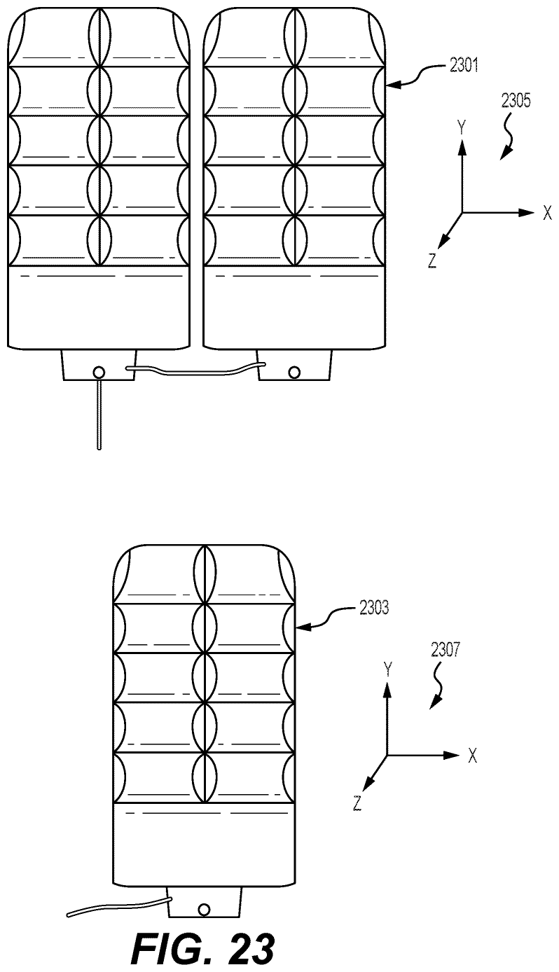

FIG. 23 is a schematic diagram for therapy electrodes used in a wearable cardiac device. As shown in FIG. 23, a therapy electrode can include two rear therapy electrodes 2301 that are configured to be positioned at a patient's back and a front therapy electrode 2303. As shown in FIG. 23, each of the therapy electrodes can be coupled to an anatomical placement sensor circuit including an inertial motion unit and the like. When assembled properly, the illustrated inertial motion units may provide signals across three axis each. In some implementations, the rear therapy electrodes are configured to be oriented perpendicular to the front therapy electrode. As such, when assembled on a patient, each of the rear therapy electrodes may oriented such that the corresponding inertial motion unit has a y-axis that points up (e.g., towards an anatomical superior direction), and a z-axis that points forward towards the front therapy electrode 2305 (e.g., towards an anatomical anterior direction). Similarly, when assembled on a patient, the front therapy electrode may be oriented such that the corresponding inertial motion unit has a y-axis that points up (e.g., towards an anatomical superior direction), and a z axis that points rearward towards the rear therapy electrode 2307 (e.g., towards an anatomical posterior direction). Determining an anatomical placement for the rear therapy electrodes 2301 and the front therapy electrode 2303 can be based on the motion signals generated by the inertial motion units attached to the respective electrodes.

To illustrate in an experimental scenario, example therapy electrodes were placed on a mannequin representative of a human torso. As shown in FIG. 23, when assembled correctly and oriented appropriately on the mannequin, “RTE” refers to rear therapy electrode and “FTE” refers to front therapy electrode. As such a relative position of the RTE to the FTE can be predetermined based on this experimental data and configured into the expected RTE and FTE placement configuration. For instance, a relative position of RTE's +y axis pointing downwards, e.g., towards an anatomical inferior direction, while the FTE's +y axis is pointing upwards, e.g., towards an anatomical superior direction, can indicate an improper placement configuration warranting further investigation.

In the experimental scenario, the RTE's can be positioned to have a y-axis that is oriented such that +y points upwards e.g., towards an anatomical superior direction, while −y points downwards e.g., towards an anatomical interior direction. The FTE's can be positioned to have a y-axis that is oriented such that the +y points upwards e.g., towards an anatomical superior direction and the −y points downwards e.g., towards an anatomical inferior direction. When the RTE is positioned perpendicular to the FTE, the corresponding RTE can be positioned to have a z-axis that is positive (or increasing) away from the wearer and negative (or decreasing) towards the FTE. The FTE can be oriented to have a z-axis that is positive (or increasing) away from the wearer and negative (or decreasing) towards the RTE.

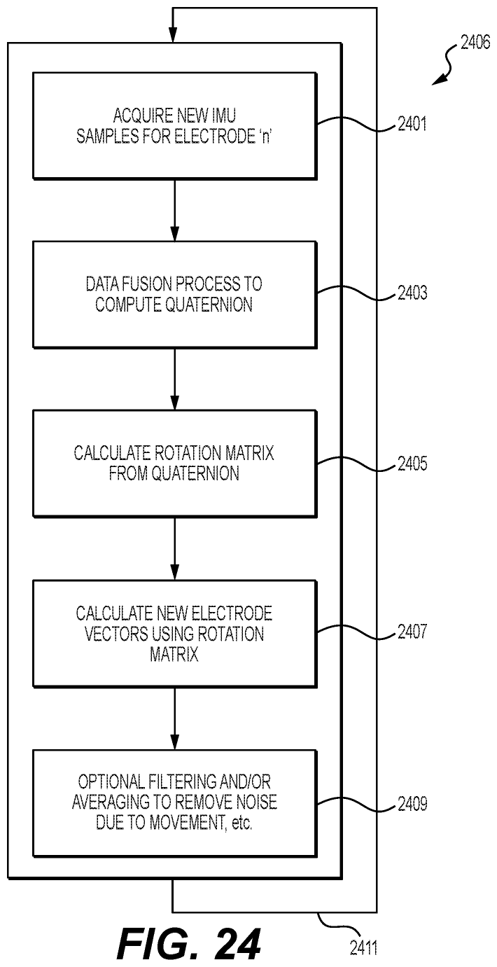

FIG. 24 provides an example of a process 2400 for processing data acquired from an inertial motion unit to generate therapy electrode location and/or orientation information. As illustrated in FIG. 24, process 2400 can include acquiring data including motion signals associated with an electrode from one or more inertial motion units 2401. The acquired data can be used to compute a quaternion using a data fusion process 2403. The quaternion may be used to calculate a rotation matrix 2405. Electrode vectors can be calculated using the rotation matrix 2407. Filters and averaging can be applied to the calculated electrode vectors in order to remove noise due to movement and the like 2409. Data fusion processes 2403 can include methods such as Mahony orientation filter, Madgwick process and the like. For example, the Mahony orientation filter can calculate an object's orientation accurately in short periods of time using axis accelerometers, 3-axis gyroscopes, and 3 axis-magnetometer to determine a quaternion orientation representation. A Madgwick algorithm can also be used in which a orientation filter is applied to IMU data consisting of tri-axial gyroscopes and accelerometers and triaxial magnetometers. The Madgwick algorithm can also utilize a quaternion representation of the orientation to describe the nature of orientations in three-dimensions. The quaternions can provide a representation of the orientations and rotations of the electrodes in three dimensional space.

As shown in FIG. 24, once a quaternion is determined, a rotation matrix can be determined 2405. The rotation matrix may represent the rotations of the electrodes in Euclidean space. The rotation matrix can be applied to the vectors corresponding to the inertial motion units associated with the electrodes in order to find their new orientation. Orientations for the sensors can be determined an repeated at a frequency. In some implementations, the orientations for the sensors can be updated at the same frequency that data from the IMUs is obtained (e.g., 50 Hz rate or the like).

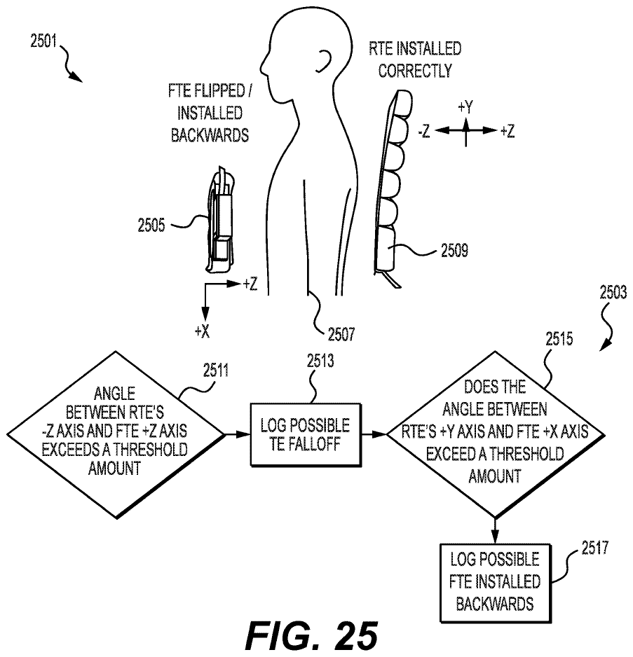

As shown in FIGS. 25-27, data from an anatomical placement motion sensor such as an inertial motion unit can be used to detect if an electrode such as a front therapy electrode or a rear therapy electrode is properly placed and installed correctly. The data can also be used to determine if one or more of the front therapy electrode and the rear therapy electrodes are flipped (i.e., facing the opposite orientation) or installed incorrectly.

FIG. 25 illustrates a process for checking if a front therapy electrode is properly installed. As shown in the schematic diagram 2501 in FIG. 25, a front therapy electrode 2505 can be flipped or installed backwards. The corresponding inertial motion units can be oriented such that the z-axis points towards the rear therapy electrodes and the x-axis points downwards. In the illustrated example the rear therapy electrodes 2509 are installed on a person 2507 correctly. The process 2503 may check if a relative positioning of a rear therapy electrode and the front therapy electrode, e.g., the angle between the rear therapy electrode's z-axis and the front therapy electrodes z axis, is more than a threshold amount of degrees 2511. If the threshold is exceeded a possible therapy electrode fall-off condition may be logged 2513. In this example, the electrode fall-off condition is triggered to indicate that an electrode is making improper or insufficient contact with the patient's skin. Subsequently, the angle formed between the rear therapy electrodes y-axis can be compared with the front therapy electrodes x-axis to determine if it exceeds a threshold amount of degrees 2515. If so, it can be determined that the front therapy electrode may be installed incorrectly (i.e., backwards).

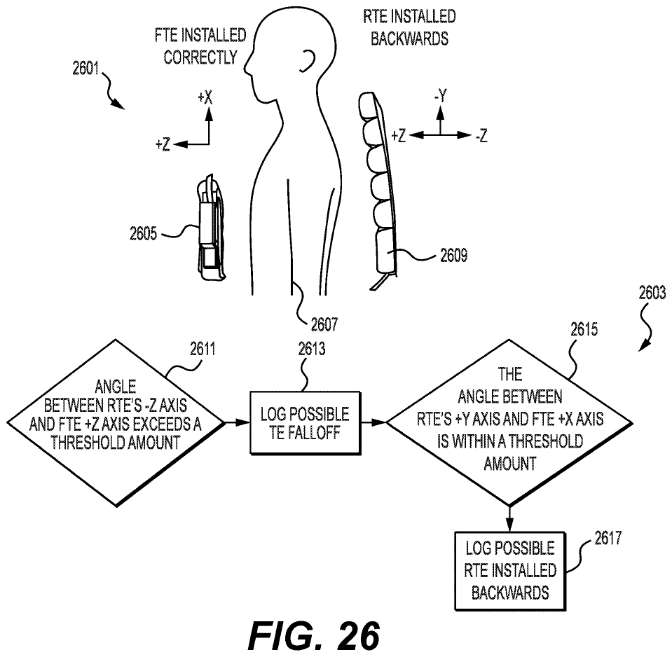

FIG. 26 illustrates a process for checking if a rear therapy electrode is properly installed. As shown in the schematic diagram 2601 in FIG. 26, a front therapy electrode 2605 can be positioned in front of a user or patient 2607. If the rear therapy electrode is flipped 2609, the positive and negative directions on the z-axis may be reversed. The process 2603 may check if the angle between the rear therapy electrode's z-axis and the front therapy electrodes z-axis is more than a threshold amount of degrees 2611. If the threshold is exceeded a possible therapy electrode fall-off condition may be logged 2613. In this example, the electrode fall-off condition is triggered to indicate that an electrode is making improper or insufficient contact with the patient's skin Subsequently, the angle formed between the rear therapy electrodes y-axis can be compared with the front therapy electrodes x-axis to determine if it exceeds a threshold amount of degrees 2615. If so, it can be determined that the rear therapy electrode may be installed incorrectly (i.e., backwards).

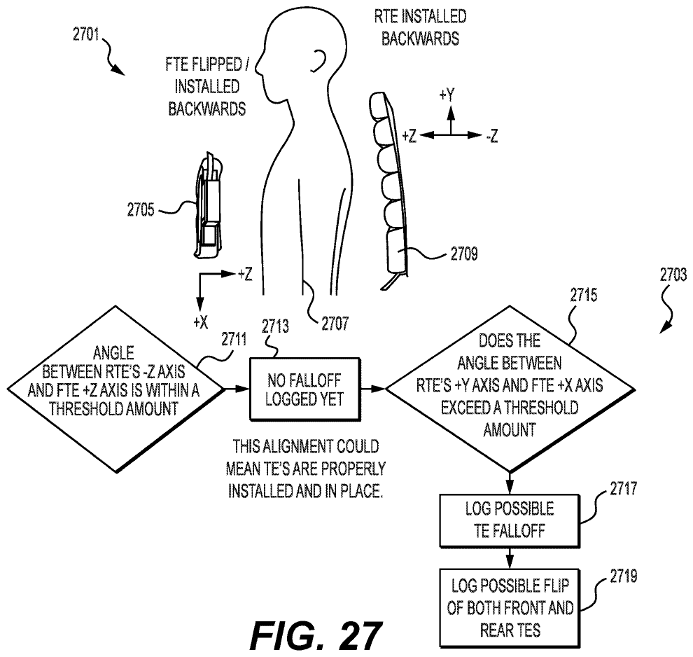

FIG. 27 illustrates a process for checking if front therapy electrode and rear therapy electrodes are properly installed. As shown in the schematic diagram 2701 in FIG. 27, a front therapy electrode 2705 can be flipped or installed backwards. The corresponding inertial motion units can be oriented such that the z-axis points towards the rear therapy electrodes and the x-axis points downwards. In the illustrated example the rear therapy electrodes 2709 are also installed on a person 2707 incorrectly. As shown, the y-axis may face upwards and the z-axis may face towards the front therapy electrodes. The process 2703 may check if the angle between the rear therapy electrode's z-axis and the front therapy electrodes z-axis is more than a threshold amount of degrees 2711. If the threshold is exceeded, it could indicate that therapy electrodes are properly installed and in place. A fall-off condition 2713 may not be logged. Subsequently, the angle formed between the rear therapy electrodes y-axis can be compared with the front therapy electrodes x-axis to determine if it exceeds a threshold amount of degrees 2715. If so, a therapy electrode fall off condition can be logged 2717. In this example, the electrode fall-off condition is triggered to indicate that an electrode is making improper or insufficient contact with the patient's skin Subsequently, it can be determined that the front therapy electrode and the rear therapy electrodes may be installed incorrectly (i.e., backwards) 2719.

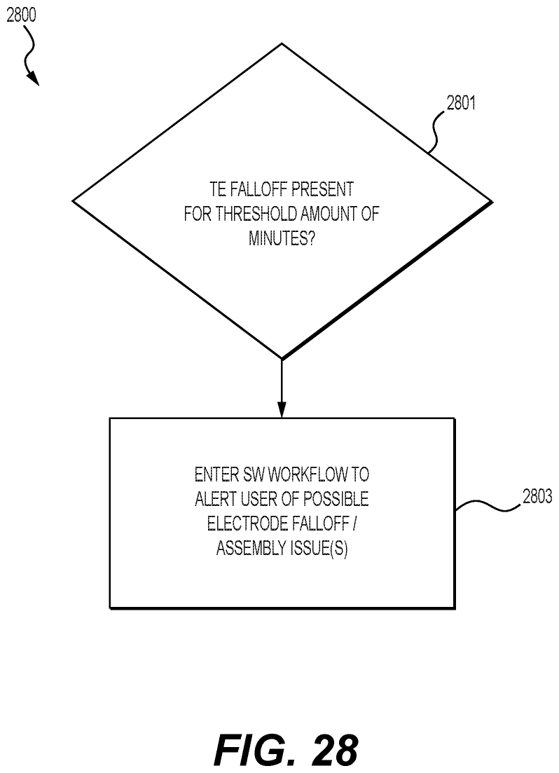

FIG. 28 illustrates a process 2800 for alerting a patient when a therapy electrode fall off is suspected. As shown in FIG. 28, it can be determined if the amount of time that the therapy electrode fall off condition is present exceeds a threshold amount 2801, and if so, a workflow can alert a user to a possible therapy electrode falloff and/or assembly issue 2803.

Accordingly, determining an improper electrode placement in garment alert can be generated when a therapy electrode falloff (e.g., an electrode is considered to be making improper or insufficient contact with the patient's skin), front therapy electrodes installed backwards, rear therapy electrode installed backwards, or flip of both front and rear therapy electrodes is logged.

In some implementations, the data from the inertial motion units and other anatomical placement motion sensor circuits can be used independently or with other methods related to electrode placement and orientation. For example, to detect fall-off conditions, in some implementations the fall-off processes described with respect to FIGS. 25-28 can be integrated with a conductive approach such as by detecting signals associated with a current sent through the body between the front and rear therapy electrodes.

In some implementations methods for determining improper electrode in garment conditions can be integrated with additional sensing circuitry. For example, if button snaps are used to secure therapy electrodes in garment pockets, the button snaps can be composed of metal and connect to the therapy electrode housing itself. Conductive material (e.g., metalized fabric or wire) installed between buttons of the garment can interface with the sensing circuitry of the therapy electrodes to indicate that the buttons are open and/or connected, thus indicating whether therapy electrodes are properly installed in the garment.

The processes disclosed herein each depict one particular sequence of acts in a particular example. The acts included in these processes may be performed by, or using, one or more computer systems specially configured as discussed herein. Some acts are optional and, as such, may be omitted in accord with one or more examples. Additionally, the order of acts can be altered, or other acts can be added, without departing from the scope of the systems and methods discussed herein. Furthermore, as discussed above, in at least one example, the acts are performed on a particular, specially configured machine, namely a medical device configured according to the examples disclosed herein.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure and are intended to be within the scope of the examples discussed herein. Accordingly, the foregoing description and drawings are by way of example only.

1. A wearable cardiac device configured for placement verification of electrodes in a garment worn by an ambulatory patient, the device comprising:
the garment configured to be worn on a torso of the ambulatory patient, the garment comprising one or more therapy electrode pockets configured to align with one or more predetermined anatomical locations on the ambulatory patient;
a plurality of ECG sensing electrodes configured to sense ECG signals of the ambulatory patient, wherein the plurality of ECG sensing electrodes are configured to be disposed at one or more predetermined ECG sensing locations within the garment;
a plurality of therapy electrodes configured to deliver one or more therapeutic shocks to the ambulatory patient, wherein each therapy electrode of the plurality of therapy electrodes is configured to be disposed within a therapy electrode pocket of the one or more therapy electrode pockets of the garment;
a plurality of anatomical placement accelerometers, wherein each anatomical placement accelerometer is integrated with a corresponding therapy electrode of the plurality of therapy electrodes and configured to generate an anatomical placement motion signal for the corresponding therapy electrode; and
a controller in electrical communication with the plurality of electrodes and the plurality of anatomical placement accelerometers, the controller configured to
receive anatomical placement motion signals from the plurality of anatomical placement accelerometers,
determine, based on the received anatomical placement motion signals, at least one of
(a) a facing orientation of the plurality of therapy electrodes, or
(b) a relative position of each therapy electrode relative to another therapy electrode of the plurality of therapy electrodes,
generate, responsive to a determination that the at least one of a facing orientation of the plurality of therapy electrodes or a relative position of each therapy electrode relative to another therapy electrode of the plurality of therapy electrodes does not meet a predetermined electrode placement criterion relating to proper placement of the electrodes within the garment, an improper electrode placement in garment alert for the patient, and
provide the generated improper electrode placement in garment alert to the patient.

2. (canceled)

3. (canceled)

4. The wearable cardiac device of claim 1, wherein the one or more therapy electrodes are configured to deliver one or more therapeutic shocks to the patient responsive to detection of a cardiac arrhythmia based on ECG signals sensed from the plurality of ECG sensing electrodes.

5. The wearable cardiac device of claim 1, wherein each of the one or more therapy electrodes is coupled to a respective ECG sensing electrode of the one or more ECG sensing electrodes to form a multifunctional electrode.

6. The wearable cardiac device of claim 1, wherein the garment comprises one or more garment attachment features for removably coupling the plurality of ECG sensing electrodes, the plurality of therapy electrodes and/or the plurality of anatomical placement accelerometers to the garment.

7. (canceled)

8. (canceled)

9. (canceled)

10. The wearable cardiac device of claim 1, further comprising a plurality of anatomical placement gyroscope sensors, wherein each anatomical placement gyroscope sensor is integrated with a corresponding anatomical placement accelerometer and configured to generate the anatomical placement motion signal for the corresponding therapy electrode.

11. (canceled)

12. The wearable cardiac device of claim 1, further comprising a plurality of anatomical placement inertial motion unit sensors, wherein each anatomical placement inertial motion unit sensor is integrated with a corresponding anatomical placement accelerometer and configured to generate the anatomical placement motion signal for the corresponding therapy electrode.

13. (canceled)

14. (canceled)

15. (canceled)

16. The wearable cardiac device of claim 151, wherein determining at least one of the facing orientation of the plurality of therapy electrodes, or the relative position of each therapy electrode relative to another therapy electrode of the plurality of therapy electrodes further comprises processing the received anatomical placement motions signal by filtering the anatomical placement motion signals to remove a contribution due to patient breathing from the anatomical placement motion signals.

17. (canceled)

18. (canceled)

19. (canceled)

20. (canceled)

21. (canceled)

22. (canceled)

23. (canceled)

24. (canceled)

25. The wearable cardiac device of claim 1 wherein the predetermined electrode placement criterion relating to proper placement of the electrodes within the garment comprises at least one of an angle, a direction, or a magnitude.

26. (canceled)

27. (canceled)

28. (canceled)

29. The device of claim 1, wherein the relative position of each therapy electrode in the plurality of therapy electrodes comprises a treatment vector for sequential shocks being applied to ambulatory patient.

30. (canceled)

31. The wearable cardiac device of claim 1, wherein the controller is further configured to:
identify at least one therapy electrode among the plurality of therapy electrodes having a corresponding anatomical placement accelerometer integrated with a corresponding therapy electrode of the plurality of therapy electrodes that does not meet the predetermined electrode placement criterion, and
disable the identified at least one therapy electrode from delivering one or more therapeutic shocks.

32. (canceled)

33. The wearable cardiac device of claim 1, further comprising a graphical user interface in electrical communication with the controller, the graphical user interface further configured to display the improper electrode placement in garment alert.

34. (canceled)

35. (canceled)

36. (canceled)

37. A wearable cardiac device configured for placement verification of electrodes on an ambulatory patient, the device comprising:
a garment configured to be worn on a torso of the ambulatory patient, the garment comprising one or more garment attachment features aligned with one or more anatomical locations on a patient;
a plurality of electrodes configured to one of sense ECG signals of a patient or deliver one or more therapeutic shocks to the patient, wherein each electrode of the plurality of electrodes is configured to be removably coupled to the garment at the one or more anatomical locations on the ambulatory patient via the one or more garment attachment features;
at least one anatomical placement sensor circuit associated with at least one of the plurality of electrodes and configured to generate at least one anatomical placement motion signal associated with the at least one of the plurality of electrodes; and
a controller in electrical communication with the plurality of electrodes and the at least one anatomical placement sensor circuit, the controller configured to
receive the generated at least one anatomical placement motion signal from the at least one anatomical placement sensor circuit;
determine, based on the received at least one anatomical placement motion signal, an anatomical placement of the associated at least one of the plurality of electrodes;
generate, responsive to a determination that the anatomical placement of the associated at least one of the plurality of electrodes does not meet an anatomical placement condition comprising a location and orientation of the plurality of electrodes within the garment, an improper electrode placement in garment alert for the patient; and
provide the generated improper electrode placement in garment alert to the patient.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The wearable cardiac device of claim 37, further comprising: removing a contribution due to patient breathing from the anatomical placement motion signal.

58. The wearable cardiac device of claim 37, wherein determining that the anatomical placement of the associated at least one of the plurality of electrodes does not meet an anatomical placement condition comprising a location and orientation of the plurality of electrodes within the garment comprises a comparison with electrode placements associated with a remainder of the plurality of electrodes.

59. (canceled)

60. (canceled)

61. The wearable cardiac device of claim 37, wherein the anatomical placement of the associated at least one of the plurality of electrodes does not meet an anatomical placement condition when a difference in a direction of the associated at least one of the plurality of electrodes and a direction of the anatomical placement condition is greater than 5% of an angle of the placement condition.

62. (canceled)

63. The device of claim 37, wherein a relative position of each electrode in the plurality of electrodes comprises a treatment vector for sequential shocks being applied to ambulatory patient.

64. The wearable cardiac device of claim 37, wherein determining the anatomical placement of the associated at least one of the plurality of electrodes comprises:
determining a four-dimensional representation of the plurality of electrodes; and
determining an electrode orientation based on the determined four-dimensional representation.

65. The wearable cardiac device of claim 37, wherein the controller is further configured to:
identify the at least one electrode among the plurality of electrodes having a corresponding anatomical placement that does not meet the anatomical placement condition; and
disable the identified at least one-electrode from delivering one or more therapeutic shocks.

66. (canceled)

67. (canceled)

68. (canceled)

69. The wearable cardiac device of claim 37, further comprising:
monitoring the patient for a determining whether a corrective action is taken; and if a corrective action has not been taken, transmitting the generated improper electrode placement in garment alert to a second party device.

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)

176. (canceled)

177. (canceled)

178. (canceled)

179. (canceled)

180. (canceled)

181. (canceled)

182. (canceled)

183. (canceled)

184. (canceled)

185. (canceled)

186. (canceled)

187. (canceled)

188. (canceled)

189. (canceled)

190. (canceled)

191. (canceled)

192. (canceled)

193. (canceled)

194. (canceled)

195. (canceled)

196. (canceled)

197. (canceled)

198. (canceled)

199. (canceled)

200. (canceled)

201. (canceled)

202. (canceled)

203. (canceled)

204. (canceled)

205. (canceled)

206. (canceled)

207. (canceled)

208. (canceled)

209. (canceled)

210. (canceled)

211. The wearable cardiac device of claim 37, wherein the garment attachment features comprise one or more of clasps, hook and loop fasteners, button and hole fasteners, and snap buttons.
